(19) United States
(12) Patent Application Publication
(10) Pub. No.: US 2026/0242730 A1
(43) Pub. Date: Aug. 20, 2026

(54) MODIFIED GAMMA DELTA T CELLS

(71) Applicant: POINTLOMA BIOSCIENCES, INC., San Diego, CA (US)

(72) Inventors: Xiaomei WANG, Pearland, TX (US); Xiaohong WANG, Houston, TX (US); Guowei WEI, San Diego, CA (US); Shuanglian LI, San Diego, CA (US); Annemarie MOSELEY, Flower Mound, TX (US); Haiyan JIANG, San Diego, CA (US)

(21) Appl. No.: 19/415,374

(22) Filed: Dec. 10, 2025

Related U.S. Application Data
(63) Continuation of application No. PCT/US2024/033421, filed on Jun. 11, 2024.
(60) Provisional application No. 63/472,509, filed on Jun. 12, 2023.

(51) Int. Cl. C12N 5/0783; C07K 14/54; C07K 16/28; C07K 16/30

(52) U.S. Cl. CPC C12N 5/0636; C07K 14/5443; C07K 16/2827; C07K 16/30; C07K 2317/31; C07K 2317/53; C07K 2317/569; C07K 2317/73; C07K 2319/02; C07K 2319/03; C12N 2510/00

(57) ABSTRACT

The technology relates in part to methods for activating and expanding multi-subsets of gamma delta T cells. In some aspects, the technology relates to multi-subsets of gamma delta T cells. In some aspects, the multi-subsets of gamma delta T cells comprise one or more chimeric antigen receptors. In some aspects, the chimeric antigen receptors comprise one or more antigen binding domains with binding specificity to TROP2 and/or PD-L1. In some aspects, the multi-subsets of gamma delta T cells express IL-15. In some aspects, the multi-subsets of gamma delta T cells comprise one or more genome modifications. In some aspects, the multi-subsets of gamma delta T cells comprise a TGFβR2 knock-out. The technology also relates in part to single-domain antibodies (nanobodies) that target TROP2, PD-L1, or TROP2 and PD-L1, uses for immune cell engineering, pharmaceutical compositions thereof, diagnostic and non-diagnostic reagents thereof, and therapeutic and non-therapeutic methods of use thereof.

### RELATED PATENT APPLICATIONS

This patent application is a continuation of International Patent Application No. PCT/US2024/033421 filed on Jun. 11, 2024, entitled MODIFIED GAMMA DELTA T CELLS, naming Xiaomei WANG et al. as inventors, and designated by attorney docket no. POINT-1001PCT, which claims the benefit of U.S. provisional patent application No. 63/472,509 filed on Jun. 12, 2023, entitled EXPANDED AND ACTIVATED POLYCLONAL GAMMA DELTA T CELLS, naming Annemarie MOSELEY et al. as inventors, and designated by attorney docket no. POINT-1001PROV1. The entire content of the foregoing patent applications is incorporated herein by reference for all purposes, including all text, tables and drawings.

### SEQUENCE LISTING

The instant application contains a Sequence Listing which has been submitted electronically in XML file format and is hereby incorporated by reference in its entirety. Said XML copy, created on Jul. 25, 2024, is named POINT-1001PCT_SL.xml and is 135,000 bytes in size.

### FIELD

The technology relates in part to methods for activating and expanding multi-subsets of gamma delta T cells. In some aspects, the technology relates to multi-subsets of gamma delta T cells. In some aspects, the technology relates to activated and expanded multi-subsets of gamma delta T cells produced by methods described herein. In some aspects, the multi-subsets of gamma delta T cells are modified. In some aspects, the multi-subsets of gamma delta T cells comprise one or more chimeric antigen receptors. In some aspects, the chimeric antigen receptors comprise one or more antigen binding domains with binding specificity to TROP2 and/or PD-L1. In some aspects, the multi-subsets of gamma delta T cells express IL-15. In some aspects, the multi-subsets of gamma delta T cells comprise one or more genome modifications. In some aspects, the multi-subsets of gamma delta T cells comprise a TGFβR2 knock-out. The technology also relates in part to single-domain antibodies (nanobodies) that target TROP2, PD-L1, or TROP2 and PD-L1, uses for immune cell engineering, pharmaceutical compositions thereof, diagnostic and non-diagnostic reagents thereof, and therapeutic and non-therapeutic methods of use thereof.

### BACKGROUND

Solid tumors present high unmet medical needs for development of new, safe, effective and accessible therapeutics. Pancreatic cancer, for example, is one of the most lethal malignant neoplasms globally, and has the 4th highest death rate for all races, ethnicities, male and female in US. Despite recent advancements, the 5-year survival rate remains at 12.5% in US. Conventional therapies such as chemotherapy and radiation have very limited effectiveness. New treatment options, such as the immune checkpoint inhibitor (ICI) pembrolizumab that is approved by the FDA for the treatment of advanced solid tumors with high microsatellite instability, has very limited application in pancreatic cancer which has very low prevalence (0.8%) of high microsatellite instability cases. ICI in combination with chemotherapy has not led to significant improvement in responses and survival.

Gamma delta T (γδ T) cells play a pivotal role in immune surveillance against tumors and pathogen-infected cells. They are a subset of T cells with distinct characteristics and functions compared to conventional αβ T cells. The γδ T cell receptor (TCR) can recognize and respond to a diverse range of antigens, including stress-induced molecules and non-peptide antigens presented on tumor cells. One of the key advantages of γδ T cells is their ability to directly recognize and target tumor cells without the need for MHC presentation of antigens. This makes γδ T cells particularly more effective than αβ T cells in highly aggressive and metastatic tumors that lost expression of MHC I as a common immune evasion mechanism that leads to loss of antigen presentation and recognition by αβ T cells. Furthermore, γδ T cells may be safely as a therapy without developing graft versus host disease.

In addition to the activation through diverse γδ TCRs, γδ T cells also respond to a wide array of ligands expressed on tumor cells through activation receptors (NKG2D and DNAM-1) and Natural Cytotoxicity Receptors (NKp30, NKp44 and NKp46), which are also expressed on NK cells, and which further enhance the breadth and amplitude of the antitumor activity of γδ T cells against heterogeneous tumor populations and antigen evasion following immunotherapy.

γδ T cells can exhibit potent cytotoxic activity directly against tumor cells, often without the need for prior priming and expansion, resulting in rapid onset of tumor killing through the release of the cytolytic molecules perforin and granzymes, and expression of death receptor ligands FASL and TRAIL that trigger tumor cell apoptosis. γδ T cells also function broadly to activate other immune cells, including: αβ T cells and dendritic cells (DC) by secreting pro-inflammatory cytokines IFN-γ and TNF-α; NK cells through 41BBL-41BB interaction; and antibody production and class switching in B cells by expressing CCR5. Vγ9Vδ2 T cells also are potent in antigen cross-presentation that efficiently stimulates αβ T cell activation and proliferation. Vγ9Vδ2 T cells also express CD16/IgG FcgRIII to mediate antibody-dependent cell-mediated cytotoxicity (ADCC).

Among the two major subtypes of γδ T cells, Vδ1 T cells are naturally tissue homing as they reside in epithelial tissue which is often the location of solid tumors. Although Vγ9Vδ2 T cells are mostly found in circulation, they also infiltrate tumor tissue through chemotaxis and activation induced by a dysregulated mevalonate pathway that leads to an accumulation of isopentenyl diphosphate (IPP) in TME. Consistently, tumor-infiltrating γδ T cells are identified to be the most favorable prognostic marker in a pan-cancer study of 5,782 tumor samples across 25 different cancer types, including 3,238 tumor samples from 14 solid non-brain tumors. Further independent studies on individual tumor types using transcriptome, flow cytometry and/or immunohistochemistry analyses of tumor infiltrating lymphocytes confirmed that γδ T cells (as in CRC, HCC, gastric cancer, head and neck squamous cell carcinoma) or Vδ1 T (in NSCLC and TNBC) or Vγ9Vδ2 T (in bladder cancer) are positively associated with disease-free survival, overall survival or early tumor stage in different solid tumors. Therefore, both tissue resident Vδ1 and circulating Vδ2 T cells can effectively infiltrate solid tumor, albeit their impact may vary between cancer types given the heterogeneity of the tumor microenvironment (TME) and immune response across different cancer types. Therapeutic multi-subsets of γδ T cells (also referred to as polyclonal γδ T cells) would have an advantage of being tissue agnostic in treating broad solid tumor types.

Solid tumors generally are highly heterogeneous and diverge early. A single target-specific treatment may lead to a clone-specific negative selection, for which the tumor has evolved numerous mechanisms to abolish therapeutic efficacy, such as antigen escape by shedding, downregulating, or silencing the antigen expression; immune escape by downregulation of MHC or loss of heterozygosity of HLA. Also, tumor associated antigens (TAA) often express on both solid tumor and normal tissues, increasing the risk of on-target off-tumor toxicity, which results in dose-limiting toxicity for many antibody-based therapeutics. Finally, the highly immune suppressive tumor microenvironment (TME) also presents a significant challenge to preserve therapeutic activity.

Monoclonal antibodies have been referred to as magic bullet drugs due to their inherent specificity. Therefore, monoclonal antibodies have a wide range of therapeutic applications. They can be used alone or as building blocks to deliver drugs, toxins, or radioactive substances directly to cancer cells. Bispecific antibody engaging (BiTE) antibodies and immune checkpoint blockade (ICB) antibodies show how antibody activity and the immune system work together to create successful targeted cancer therapies. A cancer cell-targeting antibody also can be engineered as part of a chimeric molecule (e.g., chimeric antigen receptors (CAR)) incorporated into certain cytotoxic immune cells to activate such immune cells for target-specific killing. The specificity of CAR-mediated T-cell recognition is defined by the antibody domain, is independent of MHC presentation, and can be extended to any target for which an antibody is available.

Chimeric antigen receptors (CARs) have a modular design with four major components: an antigen-binding domain, a hinge, a transmembrane domain and an intracellular signaling domain. One component of a CAR is the extracellular antibody domain, which influences the specificity and efficacy of engineered immune cells. The antigen-binding domains of CARs typically are composed of the variable heavy (VH) and variable light (VL) chains of monoclonal antibodies, connected by a flexible linker to form a single-chain variable fragment (scFv). CARs may also be engineered to contain smaller, single-domain antibodies (nanobodies) comprising the VH domain of camelid heavy-chain antibodies, also referred to as variable heavy domain of heavy chain (VHH). By comparison with conventional ScFV antibody fragments for CAR construction, single-domain antibodies not only offer comparable binding affinity but also flexibility for genetic manipulation due to their smaller size. The high binding capacity, solubility, stability, smaller size, simple humanization, and lower immunogenicity indicate the potential of VHH domain antibody in the construction of multi-specific CARs for application to heterogeneous solid tumors. In some instances, single target-specific treatment of solid tumors leads to clone-specific negative selection or immune escape by upregulation of immunological checkpoints. Immunological checkpoints are a group of inhibitory and stimulatory pathways that influence immune cell activity.

Immune checkpoint blockade (ICB) drugs targeting immune inhibitory receptors, such as CTLA-4, PD-1, and PD-L1 have been the most widely used immunotherapeutic agents and have completely transformed cancer immunotherapy. Programmed death 1 ligand 1 (PD-L1), also known as B7-H1 or CD274, is a member of the B7 family and the ligand for PD-1. PD-L1 has an effect of negative regulation on immune response. The binding of PD-L1 expressed by tumor cells and PD1 can inhibit the function of immune cells and lead to the immune escape of tumors. Certain PD-L1 inhibitors have been approved to treat some solid tumors, including NSCLC, HNSCC, melanoma, and MCC. However, only a subset (20-40%) of patients benefit from this therapy, highlighting the need for the improvement of the treatment.

As a tumor-associated antigen (TAA), PD-L1 is variably expressed across different tumor types including esophageal, gastrointestinal, pancreatic, breast, lung, and kidney cancers. Often, PD-L1 expression is strongly upregulated in response to pro-inflammatory cytokines such as INFγ and TNFα released from activated immune cells, such as adoptively transferred CAR γδ T cells. The upregulated PD-L1 on tumor cells can lead to T cell inactivation. In addition, TGF-β enriched in TME can also increase PD-L1 expression on tumor-associated-macrophages (TAM), as a mechanism for tumor immune escape. Thus, the development of CAR-T therapy dual targeting PD-L1 and other TAA may minimize not only tumor antigen evasion, but also tumor immune escape to improve the potency of CAR-T therapy.

Trophoblast cell surface antigen 2 (TROP2), also known as tumor associated calcium signal transducer 2, is a type I membrane glycoprotein that is significantly overexpressed in various solid tumors. A comprehensive analysis of 18,563 tumor samples from 150 different tumor (sub) types identified TROP2 positivity in more than 100 different tumor (sub) types. Among 86 epithelial tumor entities, 97.7% showed detectable TROP2 expression, and 90.7% showed at least one case with strong positivity. Moreover, high TROP2 expression was associated with advanced disease stages in multiple cancer types. A meta-analysis found that TROP2 overexpression was significantly associated with poor survival (overall survival (OS) or disease-free survival (DFS)) in human solid tumors, indicating that TROP2 is a valuable prognostic biomarker and a potential target for cancer therapy. Emerging evidence indicates TROP2-targeting therapies are efficacious and safe in patients with multiple prior treatments.

TROP2 can be used as a target of antibody-drug conjugate (ADC) therapy. In one example, an anti-TROP2 ADC may be used for the treatment of locally advanced or metastatic unresectable triple-negative breast cancer (TNBC), urothelial cancer and hormone receptor-positive, and HER2-negative breast cancers. Another TROP2-targeting ADC produced promising treatment results in advanced non-small cell lung cancer (NSCLC). Thus, ADCs targeting TROP2 may be useful for treating metastatic triple-negative breast cancer, urothelial cancer, and NSCLC, specifically. However, excessive toxicity, rising tumor resistance to the ADC drug payload and a low response rate of ADCs pose ongoing challenges. Safer and more effective strategies are needed for TROP2 targeting treatment. Immune cells engineered with anti-TROP2 antibody binders may be a useful treatment method for TROP2-positive cancers.

Provided herein are genetically engineered multi-subsets of γδ T cells which may be useful as a safe, effective, and accessible treatment to address high unmet medical needs in patients with wide range of solid tumor. Specifically, the modified γδ T cells described herein comprise healthy donor-derived multi-subsets of γδ T cells expressing a bispecific CAR targeting trophoblast cell surface antigen 2 (TROP2) and PD-L1, which are either widely expressed on epithelial tumors (TROP2) or upregulated in response to proinflammatory cytokines (PD-L1). PD-L1 is an immune checkpoint factor and a tumor-associated-antigen. Thus, targeting PD-L1 not only minimizes tumor antigen and immune evasion, but also averts PD-L1-mediated immune suppression and preserves the activity of the modified γδ T cells described herein. Depleting PD-L1-expressing tumor cells, stromal and immune cells in TME also disrupts the structural integrity of desmoplastic stroma and enhance lymphocytic infiltration that in turn remodels TME to be immune inflamed.

The modified γδ T cells described herein also express IL-15, which is an immunostimulatory cytokine that enhances the activity, survival, and proliferation capacity of γδ T and innate cells ex vivo and in vivo. Additionally, TGFβR2 is knocked out in the modified γδ T cells described herein (i.e., the TGFβR2 gene is edited out by CRISRP/Cas9) to overcome immune suppressive tumor microenvironment (TME) characteristics of solid tumors, especially in pancreatic cancer and other epithelial tissue cancers.

### SUMMARY

Provided in certain aspects are methods for selectively expanding gamma delta T cells ex vivo, comprising expanding the number of gamma delta T cells in an originating cell population under expansion conditions, thereby generating an expanded gamma delta T cell-enriched cell population, where the expansion conditions comprise binding agents chosen from two or more of: (i) a cluster of differentiation 3 (CD3) binding agent, (ii) a cluster of differentiation 2 (CD2) binding agent, (iii) an NKp46 binding agent, (iv) an NKp44 binding agent, and (v) an NKp30 binding agent.

Also provided in certain aspects are kits for selectively expanding gamma delta T cells ex vivo comprising binding agents chosen from two or more of: (i) a cluster of differentiation 3 (CD3) binding agent, (ii) a cluster of differentiation 2 (CD2) binding agent, (iii) an NKp46 binding agent, (iv) an NKp44 binding agent, and (v) an NKp30 binding agent.

Also provided in certain aspects are modified gamma delta T cells comprising a chimeric antigen receptor (CAR), where the CAR comprises a first antigen binding domain with binding specificity to TROP2 and a second antigen binding domain with binding specificity to PD-L1.

Also provided in certain aspects are nucleic acids encoding a chimeric antigen receptor (CAR), where the nucleic acid comprises (i) a first polynucleotide encoding a first antigen binding domain with binding specificity to TROP2, and (ii) a second polynucleotide encoding a second antigen binding domain with binding specificity to PD-L1.

Also provided in certain aspects are chimeric antigen receptors (CARs) comprising a first antigen binding domain with binding specificity to TROP2 a second antigen binding domain with binding specificity to PD-L1.

Also provided in certain aspects are pharmaceutical compositions comprising the modified gamma delta T cells described herein.

Also provided in certain aspects are methods of treatment comprising administering a therapeutic dose of the modified gamma delta T cells described herein.

Also provided herein are agents that bind TROP2, or a fragment thereof, comprising a) a hypervariable region H1 (HVR-H1) polypeptide chosen from SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, and SEQ ID NO: 90; b) a hypervariable region H2 (HVR-H2) polypeptide chosen from SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, and SEQ ID NO: 91; and c) a hypervariable region H3 (HVR-H3) polypeptide chosen from SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO:

80, SEQ ID NO: 83, SEQ ID NO: 88, and SEQ ID NO: 92.

Also provided herein are agents that bind PD-L1, or a fragment thereof, comprising a) a hypervariable region H1 (HVR-H1) polypeptide chosen from SEQ ID NO: 2, SEQ ID NO: 6, and SEQ ID NO: 10; b) a hypervariable region H2 (HVR-H2) polypeptide chosen from SEQ ID NO: 3, SEQ ID NO: 7, and SEQ ID NO: 11; and c) a hypervariable region H3 (HVR-H3) polypeptide chosen from SEQ ID NO: 4, SEQ ID NO: 8, and SEQ ID NO: 12.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIGS. 1A, 1B, 1C, 1D, 1E, 1F, and 1G show the composition, expansion and activity of gamma delta (γδ) T cells activated and expanded using anti-CD3 antibody with or without anti-CD2 antibody.

FIGS. 2A, 2B and 2C show purity of expanded γδ T cells, efficiency of TROP2-PD-L1 CAR retroviral vector transduction and TGFβR2 gRNA/Cas9-mediated gene knockout. NT: unmodified γδ T cells; CAR: TROP2-PD-L1 CAR-IL-15 γδ T cells; PLB-001: TROP2-PD-L1 CAR-IL-15, TGFβR2 KO γδ T cells. Bar graphs represent results (Mean +/−SD) from 2 healthy donors.

FIG. 3 shows the memory phenotypes as determined by CD62L and CD45RA in flow cytometry. T_{CM}: central memory; T_{NAIVE}: naïve; T_{EM}: effector memory; T_{EMRA}: terminally differentiated effector memory T cells. Activation markers: CD69+ or NKG2D+; Exhaustion markers: PD1+TIGIT+, or PD1+LAG3+. NT: unmodified γδ T cells; CAR: TROP2-PD-L1 CAR-IL-15 γδ T cells; PLB-001: TROP2-PD-L1 CAR-IL-15, TGFβR2 KO γδ T cells. Bar graphs represent results (Mean +/−SD) from 2 healthy donors.

FIG. 4 shows CAR-dependent and independent innate cytotoxicity of γδ T cells co-cultured with GFP-Luc-labeled tumor cell lines HCC1806 (TNBC), NCI-H1975 (NSCLC), BXPC3 (PDAC), and K562 (CML) at variable E:T ratio from 1:9 to 9:1. Tumor-specific lysis was measured by luciferase assay. NT: unmodified γδ T cells; CAR: TROP2-PD-L1 CAR-IL-15 γδ T cells;

FIG. 5 shows enhanced tumor killing activity of PLB-001 on broad tumor types. GFP-Luc-expressing tumor cell lines were co-cultured with specified γδ T cells derived from two healthy donors at E:T ratio of 1:2 for up to 5 days. Fluorescent cell images were taken on day 4 on BIOTEK CYTATION 5 (Agilent), and a luciferase assay was performed on day 5. Tumor luciferase activity percentage was calculated relative to tumor alone. NT: unmodified γδ T cells; CAR: TROP2-PD-L1 CAR-IL-15 γδ T cells; PLB-001: TROP2-PD-L1 CAR-IL-15, TGFβR2 KO γδ T cells. Statistical analysis was done in GRAPHPAD PRISM 9 using Paired T test. * P<0.05, ** P<0.01.

FIGS. 6A and 6B show that TGFβR2 KO preserves the tumor-killing activity of PLB-001 in the presence of TGFβ1. GFP-Luc-expressing tumor cell line BXPC3, HPAFII, NCI-H1975, or HCC1806 was co-cultured with specified γδ T cells derived from two healthy donors at ET ratio of 1:1 (Panel A) or 1:2 (Panels B, C, and D). Tumor-specific lysis was measured by luciferase assay. Levels of IFNγ, perforin, and granzyme B in the culture medium were measured by ELISA. Statistical analysis was done in GRAPHPAD PRISM 9 using Multiple Paired T test. * P<0.05, ** P<0.01. NT: unmodified γδ T cells; CAR: TROP2-PD-L1 CAR-IL-15 γδ T cells; PLB-001: TROP2-PD-L1 CAR-IL-15, TGFβR2 KO γδ T cells.

FIGS. 7, Rows A and B, show effective tumor infiltration and killing of PLB-001 in pancreatic tumor cell line-derived 3D spheroid model. The tumor spheroid was seeded with 5,000 cells of GFP-Luc-expressing BxPC3 and HPAF-II and cultured for 3 days, followed by addition of 15,000 (A) or 5,000 (B) of NT or CAR γδ T cells or PLB-001 derived from two healthy donors. Data presented are Mean +/−SD (n=4) and statistical analysis was done in GRAPHPAD PRISM 9 using Paired T test. *P<0.05, ** P<0.01. NT: unmodified γδ T cells; CAR: TROP2-PD-L1 CAR-IL-15 γδ T cells; PLB-001: TROP2-PD-L1 CAR-IL-15, TGFβR2 KO γδ T cells.

FIG. 8 shows an example of a chimeric antigen receptor (CAR) construct.

FIG. 9 shows transduction efficiency of PD-L1 CAR retroviral vectors as assessed by flow cytometry in expanded γδ T cells from two healthy donors.

FIG. 10 shows the expression of degranulation marker CD107a (upper panel) and IFN-γ (lower panel) in PD-L1 CAR γδ T cells co-cultured with tumor cells.

FIGS. 11A, 11B, and 11C show antitumor cytotoxic effects of PD-L1 CAR γδ T cells in ovarian cancer cell line SKOV3 (FIG. 11A), and lung cancer cell lines HCC827 (FIG. 11B) and NCI-H1975 (FIG. 11C).

FIGS. 12A and 12B show anti-tumor activity of PD-L1 CAR γδ T cells in 3D tumor spheroids derived from GFP-Luc-labeled lung cancer cell lines HCC827 (FIG. 12A) and NCI-H1975 (FIG. 12B).

FIGS. 13A and 13B show identification of lead phage clone binders that specifically bind to TROP2-expressing cell lines.

FIG. 14 shows efficient TROP2 CAR retroviral vector transduction of γδ T cells. Cell surface CAR expression was detected by antibody QBEND/10 specific to the CD34 tag in the CAR construct.

FIGS. 15A, 15B, and 15C show antigen binding capability of TROP2 CAR γδ T cells as determined by flow cytometry. The binding of recombinant human TROP2 protein to the displayed CAR binders on the γδ T cells was detected by anti-human FC antibody.

FIG. 16 shows EC50 of TROP2 protein against TROP2 binder-expressing γδ T cells.

FIGS. 17A, 17B, 17C, and 17D show antitumor cytotoxicity of TROP2 CAR γδ T cells cocultured with breast cancer cell line HCC1806, lung cancer cell line NCI-H1975, pancreatic cancer cell line HPAF-II, and gastric cancer cell line N87.

FIGS. 18A and 18B show tumor infiltration and killing capability of TROP2 CAR γδ T cells in tumor spheroids derived from HPAF-II and NCI-H1975 cell lines.

FIGS. 19A, 19B, and 19C show serial killing capability of TROP2 CAR γδ T cells upon repeat challenges of tumor cell lines NCI-H1975, N87, and HPAF-II.

FIG. 20A shows transduction efficiency of TROP2-PD-L1 CAR retroviral vector. FIG. 20B shows the composition of expanded TROP2-PD-L1 CAR γδ T cells.

FIGS. 21A, 21B, and 21C show antitumor cytotoxicity of TROP2-PD-L1 CAR γδ T cells cocultured with lung cancer cell lines NCI-H1975 and HCC827, and ovarian cancer cell line SKOV3.

FIGS. 22A, 2B, and 22C show antitumor activity of TROP2-PD-L1 CAR γδ T cells in tumor spheroids derived from NCI-H1975 cell line.

FIGS. 23A, 23B, 23C, and 23D show comparable cell viability, expansion and composition of multi-subsets of γδ T cells expanded with either IL-2 or IL-15

FIGS. 24A, 24B, 24C, and 24D show the efficiency of TGFβR2 KO by electroporation of TGFβR2 gRNA/Cas9 RNP in expanded γδ T cells, as measured by flow cytometry of surface TGFβR2 expression. The viability and expansion potential of γδ T cells post RNP electroporation were also assessed.

FIGS. 25A, 25B, 250, 25D, and 25E show the serial killing activity of TGFβR2 KO, TROP2-PD-L1 CAR-IL-15 γδ T cells (PLB-001) upon repeat challenges of tumor cell lines NCI-H1975, HPAFII or BXPC3. The proliferation of PLB-001 and γδ T cell subsets, as well as the exhaustion markers post repeat tumor cell exposure were also determined by flow cytometry.

FIGS. 26A, 26B, 26C, 26D, and 26E show anti-tumor activity of PLB-001 following intravenous administration in subcutaneous breast tumor xenograft model in NSG mice. Tumor and normal tissues were analyzed to determine the tissue infiltration and tumor-induced expansion of PLB-001.

### DETAILED DESCRIPTION

Provided herein are methods for activating and selectively expanding gamma delta T cells ex vivo. Also provided herein are populations of activated and expanded gamma delta T cells. In certain embodiments, gamma delta T cells are modified. For example, modifications may include introduction of a chimeric antigen receptor (CAR) and/or a genome modification. A CAR may include one or more antigen binding domains (e.g., a TROP2 antigen binding domain; a PD-L1 antigen binding domain). A CAR may include one or more cytokines (e.g., IL-15). A genome modification may include a specific gene knock-out (e.g., a TGFβR2 knock-out).

### Cell Culture

Provided herein are methods and compositions for cell culture. In particular, provided herein are expansion culture conditions (“expansion conditions”). Cell culture, or culture, typically refers to the maintenance of cells in an artificial, in vitro environment, or the maintenance of cells in an external, ex vivo environment (i.e., outside of an organism). Certain cell culture systems described herein may be an ex vivo environment and/or an in vitro environment.

Cells may be obtained from a subject and/or a cellular source. Cells obtained from a subject and/or a cellular source may be referred as an originating cell population. An originating cell population is the input population of cells for expansion by culture conditions described herein (e.g., expansion conditions). A cellular source may include a population of circulating blood cells. A cellular source may include a population of peripheral blood mononuclear cells (PBMC). A cellular source may include a population of immune cells. A cellular source may include a population of T cells. A cellular source may include a population of T cells depleted of alpha beta T cells. An originating cell population can be obtained from a subject in a variety of manners (e.g., isolated from circulation, isolated by apheresis, isolated by leukapheresis). In some embodiments, immune cells are isolated from a subject. In some embodiments, lymphocytes cells are isolated from a subject. In some embodiments, T cells are isolated from a subject. In some embodiments, cells may be derived from (e.g., obtained from) a peripheral blood mononuclear cell (PBMC) population isolated from a subject. A subject may include any animal, including but not limited to any mammal, such as mouse, rat, canine, feline, bovine, equine, porcine, non-human primate and human. In certain embodiments, a subject is a human.

Cell seeding densities may be adjusted according to certain desired culture conditions. For example, an initial seeding density of from about 1×10³ to about 1-10×10⁵ cells per cm² may be used. In some embodiments, an initial seeding density of from about 1-10 to about 1-10×10⁵ cells per cm² may be used. In certain instances, 1×10⁶ cells may be cultured in a 75 cm² culture flask. In certain instances, a cell culture bag is used. In certain instances, a membrane-based static cell culture system is used (e.g., G-REX by SCALEREADY). Cell density may be altered as needed at any passage.

Cells may be cultivated in a cell incubator at about 37° C. at normal atmospheric pressure. The incubator atmosphere may be humidified and may contain from about 3-10% carbon dioxide in the air. In some instances, the incubator atmosphere may contain from about 0.1-30% oxygen. Temperature, pressure and carbon dioxide and oxygen concentration may be altered as needed. Culture medium pH may be in the range of about 7.1 to about 7.6, or from about 7.1 to about 7.4, or from about 7.1 to about 7.3.

Cell culture medium may be replaced every 1-2 days or more or less frequently as needed. As the cells approach confluence in the culture vessel, they may be passaged. A cell passage is a splitting or dividing of the cells, and a transferring a portion of the cells into a new culture vessel or culture environment. Cells which are adherent to the cell culture surface may require detachment. Methods of detaching adherent cells from the surface of culture vessels are well known and can include the use of enzymes such as trypsin.

A single passage refers to a splitting or manual division of the cells one time, and a transfer of a smaller number of cells into a new container or environment. When passaging, the cells can be split into any ratio that allows the cells to attach and grow. For example, at a single passage the cells can be split in a 1:2 ratio, a 1:3 ratio, a 1:4 ratio, a 1:5 ratio, and so on. In some embodiments, cells are passaged at least about 1 time to at least about 300 times. For example, cells may be passaged at least about 2 times, 5 times, 10 times, 20 times, 30 times, 40 times, 50 times, 60 times, 70 times, 80 times, 90 times, 100 times, 200 times or 300 times. In some embodiments, cells are passaged at least about 15 times. In some embodiments, cells are passaged at least about 25 times.

Cell growth generally refers to cell division, such that one mother cell divides into two daughter cells. Cell growth may be referred to as cell expansion. Cell growth herein generally does not refer to an increase in the actual size (e.g., diameter, volume) of the cells. Stimulation of cell growth can be assessed by plotting cell populations (e.g., cell population doublings) over time. A cell population with a steeper growth curve generally is considered as growing faster than a cell population with a less steep curve. Growth curves can be compared for various treatments between the same cell types, or growth curves can be compared for different cell types with the same conditions, for example.

Expanding a population of cells may be expressed as population doubling. A cell population doubling occurs when the cells in culture divide so that the number of cells is doubled. In some instances, cells are counted to determine if a population of cells has doubled, tripled or multiplied by some other factor. The number of population doublings may not be equivalent to the number of times a cell culture is passaged. For example, passaging the cells and splitting them in a 1:3 ratio for further culturing may not be equivalent to a tripled cell population. A formula that may be used for the calculation of population doublings (PD) is presented in Equation A:

$$n = 3.32 * ( log Y - log I ) + X \qquad Equation A$$

where n=the final PD number of the cell culture when it is harvested or passaged, Y=the cell yield at the time of harvesting or passaging, I=the cell number used as inoculum to begin that cell culture, and X=the PD number of the originating cell culture that is used to initiate the subculture.

In some embodiments, a method herein comprises expanding a population of cells. Expanding a population of cells may be referred to as proliferating a population of cells. Expanding a population of cells may be expressed as fold increase in cell numbers. A formula that may be used for the calculation of fold increase as a function of population doublings is presented in Equation B:

$$F = {2}^{n} \qquad Equation B$$

where F=the fold increase in cell numbers after n population doublings. For example, after one (1) population doubling, the number of cells increases by 2-fold, and after two (2) population doublings, the number of cells increases by 4 (2²=4) fold, and after three (3) population doublings, the number of cells increases by 8 (2³=8) fold, and so on. Hence, after twenty (20) population doublings, the number of cells increases by more than one million-fold (2²⁰=1,048,576), and after thirty (30) population doublings, the number of cells increases by more than one billion-fold (2³⁰=1,073,741,824), and after forty (40) population doublings, the number of cells increases by more than one trillion-fold (2⁴⁰=1,099,511,627,776), and so on. In some embodiments, a population of cells is expanded, or is capable of being expanded, at least about 2-fold to at least about a trillion-fold. For example, a population of cells may be expanded at least about 5-fold, 10-fold, 15-fold, 20-fold, 30-fold, 40-fold, 50-fold, 100-fold, 200-fold, 300-fold, 1,000-fold, 10,000-fold, 100,000-fold, 1 million-fold, 1 billion-fold, or 1 trillion-fold. A particular fold expansion may occur over a certain period of time in culture such as, for example, 2 days, 3 days, 4 days, 5 days, 10 days, 20 days, 30 days, 40 days, 50 days, 100 days or more.

### Gamma Delta T Cell Expansion

Provided herein, in certain embodiments, are methods and compositions for expanding a population of gamma delta T cells. In some embodiments, methods and compositions are provided to selectively expand a population of gamma delta T cells (i.e., without or with minimal corresponding expansion of other immune cells (e.g., alpha beta T cells, NK cells)). Selective expansion of gamma delta T cells produces an expanded cell population enriched with gamma delta T cells.

In some embodiments, expansion conditions comprise one or more agents that bind to one or more antigens. A binding agent in the context of expansion condition components herein generally refers to a molecule that is or comprises one or more antibodies, antibody fragments, or antibody derivatives. In some instances, a binding agent may be a ligand. The term antibody generally encompasses a variety of antibody structures, including, but not limited to, monoclonal antibodies, polyclonal antibodies, multispecific antibodies (e.g., bispecific antibodies), and antibody fragments, so long as they exhibit the desired antigen-binding activity. An antibody fragment generally refers to a molecule other than an intact antibody that comprises a portion of the intact antibody that binds to the antigen to which the intact antibody binds. Examples of antibody fragments include, but are not limited to, Fv, Fab′-SH, F(ab′)2, and multispecific antibodies formed from antibody fragments. An antibody derivative generally refers to a molecule other than an intact antibody that comprises a portion derived from an intact antibody (or an antigen-binding fragment thereof) and which binds to an antigen to which the intact antibody (or an antigen-binding fragment thereof) binds. Examples of antibody derivatives include, but are not limited to, single chain variable fragments (scFv), VHH fragments, nanobodies, diabodies, triabodies, and the like, aptamers comprising multiple antigen-binding antibody fragments, single chain variable fragments, VHH fragments, nanobodies, diabodies, triabodies, and the like.

In some embodiments, expansion conditions herein comprise one or more agents that bind to cell surface molecules (e.g., immune cell markers). In some embodiments, expansion conditions herein comprise one or more cluster of differentiation (CD) binding agents. The cluster of differentiation (also known as cluster of designation or classification determinant) generally are cell surface markers used for immunophenotyping of cells. CD molecules can act as receptors or ligands, often initiating a signal cascade, altering the behavior of the cell. Some CD proteins do not play a role in cell signaling, but have other functions, such as cell adhesion. CD for humans is numbered from 1 to 371 (e.g., CD1 to CD371). In some embodiments, expansion conditions herein comprise a CD2 binding agent. In some embodiments, a CD2 binding agent is an antibody, antibody fragment, or antibody derivative. Any suitable anti-CD2 antibody, antibody fragment, or antibody derivative may be used in the expansion conditions herein, including commercially available anti-CD2 antibodies, antibody fragments, or antibody derivatives. In some embodiments, expansion conditions herein comprise a CD3 binding agent. In some embodiments, a CD3 binding agent is an antibody, antibody fragment, or antibody derivative. Any suitable anti-CD3 antibody, antibody fragment, or antibody derivative may be used in the expansion conditions herein, including commercially available anti-CD3 antibodies, antibody fragments, or antibody derivatives. In some embodiments, expansion conditions comprise binding agents comprising a CD3 binding agent and a CD2 binding agent. In some embodiments, expansion conditions comprise binding agents consisting of a CD3 binding agent and a CD2 binding agent.

In some embodiments, expansion conditions herein comprise one or more agents that bind to NKp44, NKp46, and/or NKp30. one or more agents that bind to NKp44 may include an NKp44 ligand (e.g., PDGF-DD). One or more agents that bind to NKp46 may include an anti-NKp46 antibody, fragment thereof, or derivative thereof. One or more agents that bind to NKp30 may include an anti-NKp30 antibody, fragment thereof, or derivative thereof. In some embodiments, expansion conditions comprise binding agents comprising a CD3 binding agent and an NKp44 binding agent. In some embodiments, expansion conditions comprise binding agents consisting of a CD3 binding agent and an NKp44 binding agent. In some embodiments, expansion conditions comprise binding agents comprising a CD3 binding agent and an NKp46 binding agent. In some embodiments, expansion conditions comprise binding agents consisting of a CD3 binding agent and an NKp46 binding agent. In some embodiments, expansion conditions comprise binding agents comprising a CD3 binding agent and an NKp30 binding agent. In some embodiments, expansion conditions comprise binding agents consisting of a CD3 binding agent and an NKp30 binding agent.

In some embodiments, expansion conditions herein comprise one or more cytokines. Cytokines generally are small proteins (~5-25 kDa) involved in cell signaling. Cytokines typically exert their functions by interacting with specific cytokine receptors on the target cell surface. Cytokines may include chemokines, interferons, interleukins, lymphokines, and tumor necrosis factors. Cytokines generally are produced by cells, including immune cells such as macrophages, B lymphocytes, T lymphocytes and mast cells, as well as endothelial cells, fibroblasts, and various stromal cells. In some embodiments, expansion conditions herein comprise one or more interleukins. Interleukins (ILs) are a group of cytokines that are expressed and secreted by white blood cells (leukocytes) as well as some other body cells. Interleukins may include IL-1 to IL-36. The majority of interleukins are synthesized by CD4 helper T-lymphocytes, as well as through monocytes, macrophages, and endothelial cells. They promote the development and differentiation of T and B lymphocytes, and hematopoietic cells. In some embodiments, expansion conditions herein comprise interleukin-15 (IL-15). In some embodiments, expansion conditions herein comprise one or more cytokines consisting of IL-15. In some embodiments, expansion conditions herein comprise one or more interleukins consisting of IL-15. In some embodiments, expansion conditions herein comprise interleukin-2 (IL-2). In some embodiments, expansion conditions herein comprise one or more cytokines consisting of IL-2. In some embodiments, expansion conditions herein comprise one or more interleukins consisting of IL-2.

In some embodiments, the number of cells is expanded several-fold in an expanded gamma delta T cell-enriched cell population when compared to the number of cells in an originating cell population. Cells being expanded a particular fold may refer to total cells in a cell population or may refer to specific cells in a population (e.g., T cells, gamma delta T cells). In some embodiments, the number of cells is expanded about 100-fold or more in an expanded gamma delta T cell-enriched cell population when compared to the number of cells in an originating cell population. In some embodiments, the number of cells is expanded about 200-fold or more in an expanded gamma delta T cell-enriched cell population when compared to the number of cells in an originating cell population. In some embodiments, the number of cells is expanded about 300-fold or more in an expanded gamma delta T cell-enriched cell population when compared to the number of cells in an originating cell population. In some embodiments, the number of cells is expanded about 400-fold or more in an expanded gamma delta T cell-enriched cell population when compared to the number of cells in an originating cell population. In some embodiments, the number of cells is expanded about 500-fold or more in an expanded gamma delta T cell-enriched cell population when compared to the number of cells in an originating cell population. In some embodiments, the number of cells is expanded about 200 to about 300-fold in an expanded gamma delta T cell-enriched cell population when compared to the number of cells in an originating cell population.

A cell population expanded by a method described herein may comprise an enriched gamma delta T cell population. For example, a cell population expanded by a method described herein may comprise about 80% or more gamma delta T cells. In some embodiments, a cell population expanded by a method described herein may comprise about 85% or more gamma delta T cells. In some embodiments, a cell population expanded by a method described herein may comprise about 90% or more gamma delta T cells. In some embodiments, a cell population expanded by a method described herein may comprise about 95% or more gamma delta T cells. In some embodiments, a cell population expanded by a method described herein may comprise about 100% gamma delta T cells. In some embodiments, a cell population expanded by a method described herein may comprise about 90% to 95% gamma delta T cells.

A cell population expanded by a method described herein may be depleted of non-gamma delta T cell. For example, a cell population expanded by a method described herein may be depleted of natural killer (NK) cells and/or alpha beta T cells. In some embodiments, an expanded gamma delta T cell-enriched cell population comprises about 5% natural killer (NK) cells or less. In some embodiments, an expanded gamma delta T cell-enriched cell population comprises about 4% natural killer (NK) cells or less. In some embodiments, an expanded gamma delta T cell-enriched cell population comprises about 3% natural killer (NK) cells or less. In some embodiments, an expanded gamma delta T cell-enriched cell population comprises about 2% natural killer (NK) cells or less. In some embodiments, an expanded gamma delta T cell-enriched cell population comprises about 1% natural killer (NK) cells or less. In some embodiments, an expanded gamma delta T cell-enriched cell population comprises no detectable natural killer (NK) cells.

In some embodiments, an expanded gamma delta T cell-enriched cell population comprises about 5% alpha beta T cells or less. In some embodiments, an expanded gamma delta T cell-enriched cell population comprises about 4% alpha beta T cells or less. In some embodiments, an expanded gamma delta T cell-enriched cell population comprises about 3% alpha beta T cells or less. In some embodiments, an expanded gamma delta T cell-enriched cell population comprises about 2% alpha beta T cells or less. In some embodiments, an expanded gamma delta T cell-enriched cell population comprises about 1% alpha beta T cells or less. In some embodiments, an expanded gamma delta T cell-enriched cell population comprises no detectable alpha beta T cells. A gamma delta T cell-enriched cell population expanded by a method described herein may comprise one or more subpopulations of gamma delta T cells. A subpopulation may comprise Vdelta1 (Vδ1) T cells, Vdelta2 (Vδ2) T cells, or non-Vδ1&Vδ2 T cells. A gamma delta T cell-enriched cell population expanded by a method described herein may comprise multi-subsets of gamma delta T cells. In some embodiments, an expanded multi-subsets of gamma delta T cell population comprise subpopulations of Vdelta1 (Vδ1) T cells and Vdelta2 (Vδ2) T cells. In some embodiments, multi-subsets of gamma delta T cells comprise subpopulations Vdelta1 (Vδ1) T cells, Vdelta2 (Vδ2) T cells, and non-Vδ1&Vδ2 T cells.

The fold expansion of each gamma delta T subpopulation may vary under the expansion conditions described herein. For example, the number of Vδ1 T cells may be expanded about 1,000-fold or more in the expanded gamma delta T cell-enriched cell population when compared to the number of cells in an originating cell population. In some embodiments, the number of Vδ1 T cells may be expanded about 2,000-fold or more in the expanded gamma delta T cell-enriched cell population when compared to the number of cells in an originating cell population. In some embodiments, the number of Vδ1 T cells may be expanded about 3,000-fold or more in the expanded gamma delta T cell-enriched cell population when compared to the number of cells in an originating cell population. In some embodiments, the number of Vδ1 T cells may be expanded about 4,000-fold or more in the expanded gamma delta T cell-enriched cell population when compared to the number of cells in an originating cell population. In some embodiments, the number of Vδ1 T cells may be expanded about 5,000-fold or more in the expanded gamma delta T cell-enriched cell population when compared to the number of cells in an originating cell population. In some embodiments, the number of Vδ1 T cells may be expanded about 6,000-fold or more in the expanded gamma delta T cell-enriched cell population when compared to the number of cells in an originating cell population. In some embodiments, the number of Vδ1 T cells may be expanded about 1,000-fold to about 6,000-fold in the expanded gamma delta T cell-enriched cell population when compared to the number of cells in an originating cell population. In some embodiments, the number of Vδ1 T cells may be expanded about 1,500-fold to about 2,500-fold in the expanded gamma delta T cell-enriched cell population when compared to the number of cells in an originating cell population. In some embodiments, the number of Vδ1 T cells may be expanded about 2,000-fold in the expanded gamma delta T cell-enriched cell population when compared to the number of cells in an originating cell population. In some embodiments, the number of Vδ1 T cells may be expanded about 10,000-fold or more in the expanded gamma delta T cell-enriched cell population when compared to the number of cells in an originating cell population. In some embodiments, the number of Vδ1 T cells may be expanded about 8,000-fold to about 12,000-fold in the expanded gamma delta T cell-enriched cell population when compared to the number of cells in an originating cell population.

In another example, the number of Vδ2 T cells may be expanded about 100-fold or more in the expanded gamma delta T cell-enriched cell population when compared to the number of cells in an originating cell population. In some embodiments, the number of Vδ2 T cells may be expanded about 200-fold or more in the expanded gamma delta T cell-enriched cell population when compared to the number of cells in an originating cell population. In some embodiments, the number of Vδ2 T cells may be expanded about 300-fold or more in the expanded gamma delta T cell-enriched cell population when compared to the number of cells in an originating cell population. In some embodiments, the number of Vδ2 T cells may be expanded about 400-fold or more in the expanded gamma delta T cell-enriched cell population when compared to the number of cells in an originating cell population. In some embodiments, the number of Vδ2 T cells may be expanded about 500-fold or more in the expanded gamma delta T cell-enriched cell population when compared to the number of cells in an originating cell population. In some embodiments, the number of Vδ2 T cells may be expanded about 600-fold or more in the expanded gamma delta T cell-enriched cell population when compared to the number of cells in an originating cell population. In some embodiments, the number of Vδ2 T cells may be expanded about 700-fold or more in the expanded gamma delta T cell-enriched cell population when compared to the number of cells in an originating cell population. In some embodiments, the number of Vδ2 T cells may be expanded about 800-fold or more in the expanded gamma delta T cell-enriched cell population when compared to the number of cells in an originating cell population. In some embodiments, the number of Vδ2 T cells may be expanded about 900-fold or more in the expanded gamma delta T cell-enriched cell population when compared to the number of cells in an originating cell population. In some embodiments, the number of Vδ2 T cells may be expanded about 1,000-fold or more in the expanded gamma delta T cell-enriched cell population when compared to the number of cells in an originating cell population. In some embodiments, the number of Vδ2 T cells may be expanded about 100-fold to about 1,400-fold in the expanded gamma delta T cell-enriched cell population when compared to the number of cells in an originating cell population. In some embodiments, the number of Vδ2 T cells may be expanded about 200-fold to about 1,000-fold in the expanded gamma delta T cell-enriched cell population when compared to the number of cells in an originating cell population. In some embodiments, the number of Vδ2 T cells may be expanded about 600-fold to about 800-fold in the expanded gamma delta T cell-enriched cell population when compared to the number of cells in an originating cell population. In some embodiments, the number of Vδ2 T cells may be expanded about 700-fold in the expanded gamma delta T cell-enriched cell population when compared to the number of cells in an originating cell population. In some embodiments, the number of Vδ2 T cells may be expanded about 300-fold to 400-fold in the expanded gamma delta T cell-enriched cell population when compared to the number of cells in an originating cell population. In some embodiments, the number of Vδ2 T cells may be expanded about 350-fold in the expanded gamma delta T cell-enriched cell population when compared to the number of cells in an originating cell population.

In some embodiments, an expanded gamma delta T cell population comprises varying amounts of subpopulations. Amounts (e.g., percentages) may refer to the percent of gamma delta T cells or may refer to the percent of total cells in the population. For example, an expanded gamma delta T cell population may comprise between about 5-60% Vδ1 T cells. In some embodiments, an expanded gamma delta T cell population comprises between about 10-60% Vδ1 T cells. In some embodiments, an expanded gamma delta T cell population comprises between about 20-60% Vδ1 T cells. In some embodiments, an expanded gamma delta T cell population comprises between about 30-60% Vδ1 T cells. In some embodiments, an expanded gamma delta T cell population comprises between about 40-60% Vδ1 T cells. In some embodiments, an expanded gamma delta T cell population comprises between about 50-55% Vδ1 T cells. In some embodiments, an expanded gamma delta T cell population comprises between about 53-54% Vδ1 T cells.

In some embodiments, an expanded gamma delta T cell population comprises between about 25-95% Vδ2 T cells. In some embodiments, an expanded gamma delta T cell population comprises between about 25-85% Vδ2 T cells. In some embodiments, an expanded gamma delta T cell population comprises between about 25-75% Vδ2 T cells. In some embodiments, an expanded gamma delta T cell population comprises between about 25-65% Vδ2 T cells. In some embodiments, an expanded gamma delta T cell population comprises between about 25-55% Vδ2 T cells. In some embodiments, an expanded gamma delta T cell population comprises between about 25-45% Vδ2 T cells. In some embodiments, an expanded gamma delta T cell population comprises between about 25-35% Vδ2 T cells. In some embodiments, an expanded gamma delta T cell population comprises between about 28-33% Vδ2 T cells. In some embodiments, an expanded gamma delta T cell population comprises between about 30-31% Vδ2 T cells. In some embodiments, an expanded gamma delta T cell population comprises between about 5-15% non-Vδ1&Vδ2 T cells. In some embodiments, an expanded gamma delta T cell population comprises between about 6-11% non-Vδ1&Vδ2 T cells. In some embodiments, an expanded gamma delta T cell population comprises between about 8-9% non-Vδ1&Vδ2 T cells.

In some embodiments, a method herein comprises isolating the gamma delta T cells from the expanded gamma delta T cell-enriched cell population. In some embodiments, a method herein comprises modifying the gamma delta T cells in the expanded gamma delta T cell-enriched cell population. Expanded gamma delta T cells may be modified (e.g., genetically engineered) by a modification described herein (e.g., introduction of a CAR, genome modification). In some embodiments, a method herein comprises storing the expanded gamma delta T cell-enriched cell population or isolate thereof in a cell bank. In some embodiments, gamma delta T cells described herein may provide banks of clinical grade cells that can be used in a number of patients. In some embodiments, a bank is suitably populated with gamma delta T cells obtained from healthy volunteer donors (e.g., of blood group O). Collected and processed gamma delta T cells described herein may be banked for future use at a cell bank or depository. Accordingly, the cells may be stored in a cryoprotectant such as DMSO or CryoStor™ and subjected to a controlled rate of freezing and storage in liquid nitrogen. Gamma delta T cells may be stored in a unitized storage of defined units or dosages as required for a single or multiple treatment steps.

### Chimeric Antigen Receptor (CAR)

Provided herein are chimeric antigen receptors (CARs). In some embodiments, a modified gamma delta T cell comprises a CAR. A CAR generally refers to a chimeric polypeptide which comprises one or more polypeptide components that recognize one or more target antigens (extracellular domain; antigen recognition domains; antigen binding domains) linked to a transmembrane polypeptide and intracellular domain polypeptide selected to activate T cells. The antigen-recognition domain may be an antibody component (e.g., single-chain variable fragment (ScFv), antigen binding fragment of heavy chain only antibodies (VHH fragment), nanobody, and the like), or may, for example, be derived from other molecules such as, for example, a T cell receptor or pattern recognition receptor. The intracellular domain typically comprises at least one polypeptide which causes activation of the T cell, such as, for example, but not limited to, CD3 zeta, and, for example, co-stimulatory domains, for example, but not limited to, CD28, OX40, CD40L, ICOS, CD27, NKG2D, DNAM-1, DAP10, DAP12, and 4-1BB. A CAR may also refer to chimeric receptors that are not derived from antibodies, but are chimeric T cell receptors. Chimeric T cell receptors may comprise a polypeptide sequence that recognizes a target antigen, where the recognition sequence may be, for example, but not limited to, the recognition sequence derived from a T cell receptor or an scFv. The intracellular domain polypeptides are those that act to activate the T cell.

A CAR described herein may comprise one or more antigen binding domains. An antigen binding domain may specifically bind to a target. The terms specifically bind, specific for, and like terms generally refer to a molecule or domain (e.g., antigen binding domain) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antigen binding domain that specifically binds a target will typically bind the target with at least a 2-fold greater affinity than a non-target. Specificity can be determined using standard methods, e.g., solid-phase ELISA immunoassays. The term binds with respect to a target (e.g., antigen, analyte, immune complex), typically indicates that an antigen binding domain binds a majority of the targets in a pure population (assuming appropriate molar ratios). For example, an antigen binding domain that binds a given target typically binds to at least about ⅔ of the particular targets in a solution (e.g., at least about 75%, 80%, 85%, 90%, 91%, 92%, 93%, 94%, 95%, 96%, 97%, 98%, 99%, or 100% of the particular targets in a solution).

In some embodiments, a CAR described herein includes a VH-VL dimer or single chain antibody (antibody that exists as a single polypeptide chain), or fragment thereof, such as a single chain Fv antibody (sFv or scFv) in which a variable heavy domain and a variable light domain are joined together (directly or through a peptide linker) to form a continuous polypeptide. A single chain Fv antibody typically is a covalently linked VH-VL which may be expressed from a nucleic acid including VH- and VL-encoding sequences either joined directly or joined by a peptide-encoding linker. While the VH and VL are connected to each other as a single polypeptide chain, the VH and VL domains typically associate non-covalently. VH and VL domains together typically include six hypervariable regions (HVRs) (also referred to as complementarity determining regions (CDRs)) (three in each from the heavy chain and light chain) that contribute amino acid residues for antigen binding and confer antigen binding specificity to the antibody. In certain instances, a single variable domain (or half of an Fv comprising only three HVRs specific for an antigen) has the ability to recognize and bind a target. A dsFv is an Fv with an engineered intermolecular disulfide bond, which stabilizes the VH-VL pair. In some embodiments, a CAR described herein includes a single-domain antibody or fragment thereof engineered from heavy-chain antibodies (e.g., heavy-chain antibodies found in camelids), referred to as VHH fragments. VHH fragments typically include three hypervariable regions (HVRs) (also referred to as complementarity determining regions (CDRs)): hypervariable region H1 (HVR-H1), hypervariable region H2 (HVR-H2), and hypervariable region H3 (HVR-H3).

In some embodiments, an antigen binding domain comprises one or more variations (e.g., amino acid substitutions, deletions, and/or insertions). An example method for identification of certain residues or regions of an antigen binding domain that are preferred locations for amino acid substitutions is alanine scanning mutagenesis. Here, a residue or group of target residues are identified (e.g., charged residues such as arg, asp, his, lys, and glu) and replaced by a neutral or negatively charged amino acid (most preferably alanine or polyalanine) to affect the interaction of the amino acids with an antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at a target codon or region and the expressed antigen binding domain variants may be screened for the desired activity. Amino acid sequence insertions may include amino-terminal and/or carboxyl-terminal fusions ranging in length from one residue to polypeptides containing a hundred or more residues, as well as intra-sequence insertions of single or multiple amino acid residues. Examples of terminal insertions include an N-terminal methionyl residue.

Sites for amino acid substitutions may include sites in the hypervariable regions and may include sites in the framework regions. Amino acid substitutions may include conservative substitutions or non-conservative substitutions. Examples of substitutions are listed below.
Ala (A): val; leu; ile
Arg (R): lys; gln; asn
Asn (N): gln; his; asp, lys; arg
Asp (D): glu; asn
Cys (C): ser; ala
Gin (Q): asn; glu
Glu (E): asp; gln
Gly (G): ala
His (H): asn; gln; lys; arg
IIe (I): leu; val; met; ala; phe; norleucine
Leu (L): norleucine; ile; val; met; ala; phe
Lys (K): arg; gln; asn
Met (M): leu; phe; ile
Phe (F): leu; val; ile; ala; tyr
Pro (P): ala
Ser(S): thr
Thr (T): ser
Trp (W): tyr; phe
Tyr (Y): trp; phe; thr; ser
Val (V): ile; leu; met; phe; ala; norleucine

Substantial modifications in the biological properties of an antigen binding domain may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Naturally occurring residues can be divided into groups based on common side-chain properties listed below.
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions generally entail exchanging a member of one of these classes for another class.

In some embodiments, a CAR comprises a TROP2 antigen binding domain (e.g., an anti-TROP2 VHH polypeptide; an anti-TROP2 ScFv polypeptide). In some embodiments, a CAR comprises a human TROP2 antigen binding domain. In some embodiments, a CAR comprises a PD-L1 antigen binding domain (e.g., an anti-PD-L1 VHH polypeptide; an anti-PD-L1 ScFv polypeptide). In some embodiments, a CAR comprises a human PD-L1 antigen binding domain. In some embodiments, a

CAR comprises a TROP2 antigen binding domain (e.g., an anti-TROP2 VHH polypeptide; an anti-TROP2 ScFv polypeptide) and a PD-L1 antigen binding domain (e.g., an anti-PD-L1 VHH polypeptide; an anti-PD-L1 ScFv polypeptide). Antigen binding domains herein may be referred to as a first antigen binding domain and a second antigen binding domain, however, “first” and “second” do not limit the order of each domain in a CAR. Accordingly, a “first” antigen domain may appear first or second in a CAR construct and a “second” antigen binding domain may appear first or second in a CAR construct.

In some embodiments, A CAR comprises an extracellular domain, where the extracellular domain comprises a TROP2 antigen binding domain, a linker, a PD-L1 antigen binding domain, and a hinge. In some embodiments, an extracellular domain comprises a PD-L1 antigen binding domain, a linker, a TROP2 antigen binding domain, and a hinge. In some embodiments, an extracellular domain comprises a plurality of TROP2 antigen binding domains. In some embodiments, an extracellular domain comprises a plurality of PD-L1 antigen binding domains. For example, an extracellular domain may comprise two TROP2 antigen binding domains and one PD-L1 antigen binding domain. An extracellular domain may comprise one TROP2 antigen binding domain and two PD-L1 antigen binding domains. An extracellular domain may comprise two TROP2 antigen binding domains and two PD-L1 antigen binding domains. The plurality of PD-L1 antigen binding domains and/or the plurality of TROP2 antigen binding domains may be present in the extracellular domain in any order.

In some embodiments, a CAR comprises a polypeptide chosen from the polypeptides in Table 4 (e.g., SEQ ID NO: 102, SEQ ID NO: 103, SEQ ID NO: 104, SEQ ID NO: 105, SEQ ID NO: 106, SEQ ID NO: 107). In some embodiments, a CAR comprises a polypeptide that is at least about 80% identical to a polypeptide chosen from SEQ ID NO: 102, SEQ ID NO: 103, SEQ ID NO: 104, SEQ ID NO: 105, SEQ ID NO: 106, and SEQ ID NO: 107. In some embodiments, a CAR comprises a polypeptide that is at least about 85% identical to a polypeptide chosen from SEQ ID NO: 102, SEQ ID NO: 103, SEQ ID NO: 104, SEQ ID NO: 105, SEQ ID NO: 106, and SEQ ID NO: 107. In some embodiments, a CAR comprises a polypeptide that is at least about 90% identical to a polypeptide chosen from SEQ ID NO: 102, SEQ ID NO: 103, SEQ ID NO: 104, SEQ ID NO: 105, SEQ ID NO: 106, and SEQ ID NO: 107. In some embodiments, a CAR comprises a polypeptide that is at least about 91%, 92%, 93%, 94%, 95%, 96%, 97%, 98%, or 99% identical to a polypeptide chosen from SEQ ID NO: 102, SEQ ID NO: 103, SEQ ID NO: 104, SEQ ID NO: 105, SEQ ID NO: 106, and SEQ ID NO: 107.

In certain configurations, a CAR comprises an anti-PD-L1 VHH polypeptide. In certain configurations, a CAR comprises an anti-PD-L1 VHH polypeptide chosen from the VHH polypeptides in Table 1 (e.g., SEQ ID NO: 1, SEQ ID NO: 5, SEQ ID NO: 9). In certain configurations, a CAR comprises an anti-PD-L1 VHH polypeptide comprising an amino acid sequence that is at least about 80% identical to an anti-PD-L1 VHH amino acid sequence in Table 1. In certain configurations, a CAR comprises an anti-PD-L1 VHH polypeptide comprising an amino acid sequence that is at least about 85% identical to an anti-PD-L1 VHH amino acid sequence in Table 1. In certain configurations, a CAR comprises an anti-PD-L1 VHH polypeptide comprising an amino acid sequence that is at least about 90% identical to an anti-PD-L1 VHH amino acid sequence in Table 1. In certain configurations, a CAR comprises an anti-PD-L1 VHH polypeptide comprising an amino acid sequence that is at least about 91%, 92%, 93%, 94%, 95%, 96%, 97%, 98%, or 99% identical to an anti-PD-L1 VHH amino acid sequence in Table 1.

In certain configurations, a CAR comprises an anti-PD-L1 VHH polypeptide comprising a hypervariable region H1 (HVR-H1) chosen from the HVR-H1 polypeptides in Table 1 (e.g., SEQ ID NO: 2, SEQ ID NO: 6, SEQ ID NO: 10). In some embodiments, an anti-PD-L1 VHH polypeptide provided herein comprises an HVR-H1 polypeptide that is at least 80 percent identical to an HVR-H1 polypeptide in Table 1 (e.g., SEQ ID NO: 2, SEQ ID NO: 6, SEQ ID NO: 10). In some embodiments, an anti-PD-L1 VHH polypeptide provided herein comprises an HVR-H1 polypeptide that is at least 85 percent identical to an HVR-H1 polypeptide in Table 1 (e.g., SEQ ID NO: 2, SEQ ID NO: 6, SEQ ID NO: 10). In some embodiments, an anti-PD-L1 VHH polypeptide provided herein comprises an HVR-H1 polypeptide that is at least 90 percent identical to an HVR-H1 polypeptide in Table 1 (e.g., SEQ ID NO: 2, SEQ ID NO: 6, SEQ ID NO: 10). In some embodiments, an anti-PD-L1 VHH polypeptide provided herein comprises an HVR-H1 polypeptide that is at least about 91%, 92%, 93%, 94%, 95%, 96%, 97%, 98%, or 99% identical to an HVR-H1 polypeptide in Table 1 (e.g., SEQ ID NO: 2, SEQ ID NO: 6, SEQ ID NO: 10).

In some embodiments, a CAR comprises an anti-PD-L1 VHH polypeptide comprising a hypervariable region H1 (HVR-H1) chosen from the HVR-H1 polypeptides in Table A.

TABLE A
Anti-PD-L1 VHH HVR sequences

| SEQ ID | | |
|---|---|---|
| NO | Notes | Sequences |
| | anti-PD-L1, | X₁S X₂ X₃ X₄I X₅V |
| | HVR-H1 polypeptide | X₁ = S/E |
| | | X₂ = I/V |
| | | X₃ = A/F |
| | | X₄ = S/N |
| | | X₅ = N/A |
| 135 | anti-PD-L1, | G X₁ X₂ X₃NHYG |
| | HVR-H1 polypeptide | X₁ = F/R/S |
| | | X₂ = W/T/S/R |
| | | X₃ = F/L |
| 136 | anti-PD-L1, | G X₁ X₂ X₃SLFE |
| | HVR-H1 polypeptide | X₁ = F/R/S |
| | | X₂ = W/T/S/R |
| | | X₃ = F/L |

In certain configurations, a CAR comprises an anti-PD-L1 VHH polypeptide comprising a hypervariable region H2 (HVR-H2) chosen from the HVR-H2 polypeptides in Table 1 (e.g., SEQ ID NO: 3, SEQ ID NO: 7, SEQ ID NO: 11). In some embodiments, an anti-PD-L1 VHH polypeptide provided herein comprises an HVR-H2 polypeptide that is at least 80 percent identical to an HVR-H2 polypeptide in Table 1 (e.g., SEQ ID NO: 3, SEQ ID NO: 7, SEQ ID NO: 11). In some embodiments, an anti-PD-L1 VHH polypeptide provided herein comprises an HVR-H2 polypeptide that is at least 85 percent identical to an HVR-H2 polypeptide in Table 1 (e.g., SEQ ID NO: 3, SEQ ID NO: 7, SEQ ID NO: 11). In some embodiments, an anti-PD-L1 VHH polypeptide provided herein comprises an HVR-H2 polypeptide that is at least 90 percent identical to an HVR-H2 polypeptide in Table 1 (e.g., SEQ ID NO: 3, SEQ ID NO: 7, SEQ ID NO: 11). In some embodiments, an anti-PD-L1 VHH polypeptide provided herein comprises an HVR-H2 polypeptide that is at least about 91%, 92%, 93%, 94%, 95%, 96%, 97%, 98%, or 99% identical to an HVR-H2 polypeptide in Table 1 (e.g., SEQ ID NO: 3, SEQ ID NO: 7, SEQ ID NO: 11).

In certain configurations, a CAR comprises an anti-PD-L1 VHH polypeptide comprising a hypervariable region H3 (HVR-H3) chosen from the HVR-H3 polypeptides in Table 1 (e.g., SEQ ID NO: 4, SEQ ID NO: 8, SEQ ID NO: 12). In some embodiments, an anti-PD-L1 VHH polypeptide provided herein comprises an HVR-H3 polypeptide that is at least 80 percent identical to an HVR-H3 polypeptide in Table 1 (e.g., SEQ ID NO: 4, SEQ ID NO: 8, SEQ ID NO: 12). In some embodiments, an anti-PD-L1 VHH polypeptide provided herein comprises an HVR-H3 polypeptide that is at least 85 percent identical to an HVR-H3 polypeptide in Table 1 (e.g., SEQ ID NO: 4, SEQ ID NO: 8, SEQ ID NO: 12. In some embodiments, an anti-PD-L1 VHH polypeptide provided herein comprises an HVR-H3 polypeptide that is at least 90 percent identical to an HVR-H3 polypeptide in Table 1 (e.g., SEQ ID NO: 4, SEQ ID NO: 8, SEQ ID NO: 12). In some embodiments, an anti-PD-L1 VHH polypeptide provided herein comprises an HVR-H3 polypeptide that is at least about 91%, 92%, 93%, 94%, 95%, 96%, 97%, 98%, or 99% identical to an HVR-H3 polypeptide in Table 1 (e.g., SEQ ID NO: 4, SEQ ID NO: 8, SEQ ID NO: 12).

In certain configurations, a CAR comprises an anti-TROP2 VHH polypeptide. In certain configurations, a CAR comprises an anti-TROP2 VHH polypeptide chosen from the VHH polypeptides in Table 2 (e.g., SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, SEQ ID NO: 89). In certain configurations, a CAR comprises an anti-TROP2 VHH polypeptide comprising an amino acid sequence that is at least about 80% identical to an anti-TROP2 VHH amino acid sequence in Table 2. In certain configurations, a CAR comprises an anti-TROP2 VHH polypeptide comprising an amino acid sequence that is at least about 85% identical to an anti-TROP2 VHH amino acid sequence in Table 2. In certain configurations, a CAR comprises an anti-TROP2 VHH polypeptide comprising an amino acid sequence that is at least about 90% identical to an anti-TROP2 VHH amino acid sequence in Table 2. In certain configurations, a CAR comprises an anti-TROP2 VHH polypeptide comprising an amino acid sequence that is at least about 91%, 92%, 93%, 94%, 95%, 96%, 97%, 98%, or 99% identical to an anti-TROP2 VHH amino acid sequence in Table 2.

In certain configurations, a CAR comprises an anti-TROP2 VHH polypeptide comprising a hypervariable region H1 (HVR-H1) chosen from the HVR-H1 polypeptides in Table 2 (e.g., SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90). In some embodiments, an anti-TROP2 VHH polypeptide provided herein comprises an HVR-H1 polypeptide that is at least 80 percent identical to an HVR-H1 polypeptide in Table 2 (e.g., SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90). In some embodiments, an anti-TROP2 VHH polypeptide provided herein comprises an HVR-H1 polypeptide that is at least 85 percent identical to an HVR-H1 polypeptide in Table 2 (e.g., SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90). In some embodiments, an anti-TROP2 VHH polypeptide provided herein comprises an HVR-H1 polypeptide that is at least 90 percent identical to an HVR-H1 polypeptide in Table 2 (e.g., SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90). In some embodiments, an anti-TROP2 VHH polypeptide provided herein comprises an HVR-H1 polypeptide that is at least about 91%, 92%, 93%, 94%, 95%, 96%, 97%, 98%, or 99% identical to an HVR-H1 polypeptide in Table 2 (e.g., SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, SEQ ID NO: 90).

In certain configurations, a CAR comprises an anti-TROP2 VHH polypeptide comprising a hypervariable region H2 (HVR-H2) chosen from the HVR-H2 polypeptides in Table 2 (e.g., SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, SEQ ID NO: 91). In some embodiments, an anti-TROP2 VHH polypeptide provided herein comprises an HVR-H2 polypeptide that is at least 80 percent identical to an HVR-H2 polypeptide in Table 2 (e.g., SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, SEQ ID NO: 91). In some embodiments, an anti-TROP2 VHH polypeptide provided herein comprises an HVR-H2 polypeptide that is at least 85 percent identical to an HVR-H2 polypeptide in Table 2 (e.g., SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, SEQ ID NO: 91). In some embodiments, an anti-TROP2 VHH polypeptide provided herein comprises an HVR-H2 polypeptide that is at least 90 percent identical to an HVR-H2 polypeptide in Table 2 (e.g., SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, SEQ ID NO: 91). In some embodiments, an anti-TROP2 VHH polypeptide provided herein comprises an HVR-H2 polypeptide that is at least about 91%, 92%, 93%, 94%, 95%, 96%, 97%, 98%, or 99% identical to an HVR-H2 polypeptide in Table 2 (e.g., SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, SEQ ID NO: 91).

In certain configurations, a CAR comprises an anti-TROP2 VHH polypeptide comprising a hypervariable region H3 (HVR-H3) chosen from the HVR-H3 polypeptides in Table 2 (e.g., SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, SEQ ID NO: 92). In some embodiments, an anti-TROP2 VHH polypeptide provided herein comprises an HVR-H3 polypeptide that is at least 80 percent identical to an HVR-H3 polypeptide in Table 2 (e.g., SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, SEQ ID NO: 92). In some embodiments, an anti-TROP2 VHH polypeptide provided herein comprises an HVR-H3 polypeptide that is at least 85 percent identical to an HVR-H3 polypeptide in Table 2 (e.g., SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, SEQ ID NO: 92). In some embodiments, an anti-TROP2 VHH polypeptide provided herein comprises an HVR-H3 polypeptide that is at least 90 percent identical to an HVR-H3 polypeptide in Table 2 (e.g., SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, SEQ ID NO: 92). In some embodiments, an anti-TROP2 VHH polypeptide provided herein comprises an HVR-H3 polypeptide that is at least about 91%, 92%, 93%, 94%, 95%, 96%, 97%, 98%, or 99% identical to an HVR-H3 polypeptide in Table 2 (e.g., SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, SEQ ID NO: 92).

In certain configurations, a CAR comprises an anti-TROP2 ScFv. In some embodiments, an anti-TROP2 ScFv comprises a polypeptide that is at least about 80% identical to the polypeptide of SEQ ID NO: 101. In some embodiments, an anti-TROP2 ScFv comprises a polypeptide that is at least about 85% identical to the polypeptide of SEQ ID NO: 101. In some embodiments, an anti-TROP2 ScFv comprises a polypeptide that is at least about 90% identical to the polypeptide of SEQ ID NO: 101. In some embodiments, an anti-TROP2 ScFv comprises a polypeptide that is at least about 91%, 92%, 93%, 94%, 95%, 96%, 97%, 98%, or 99% identical to the polypeptide of SEQ ID NO: 101. In some embodiments, an anti-TROP2 ScFv comprises the polypeptide of SEQ ID NO: 101.

In certain configurations, a CAR comprises an anti-TROP2 ScFv, where the ScFv comprises a variable heavy chain (VH) domain and a variable light chain (VL) domain. In some embodiments, a VH domain comprises a polypeptide that is at least about 80% identical to the polypeptide of SEQ ID NO: 93. In some embodiments, a VH domain comprises a polypeptide that is at least about 85% identical to the polypeptide of SEQ ID NO: 93. In some embodiments, a VH domain comprises a polypeptide that is at least about 90% identical to the polypeptide of SEQ ID NO: 93. In some embodiments, a VH domain comprises a polypeptide that is at least about 91%, 92%, 93%, 94%, 95%, 96%, 97%, 98%, or 99% identical to the polypeptide of SEQ ID NO: 93. In some embodiments, a VH domain comprises the polypeptide of SEQ ID NO: 93. In some embodiments, a VL domain comprises a polypeptide that is at least about 80% identical to the polypeptide of SEQ ID NO: 97. In some embodiments, a VL domain comprises a polypeptide that is at least about 85% identical to the polypeptide of SEQ ID NO: 97. In some embodiments, a VL domain comprises a polypeptide that is at least about 90% identical to the polypeptide of SEQ ID NO: 97. In some embodiments, a VL domain comprises a polypeptide that is at least about 91%, 92%, 93%, 94%, 95%, 96%, 97%, 98%, or 99% identical to the polypeptide of SEQ ID NO: 97. In some embodiments, a VL domain comprises the polypeptide of SEQ ID NO: 97.

An ScFv VH domain may comprise a hypervariable region H1 (HVR-H1) polypeptide, a hypervariable region H2 (HVR-H2) polypeptide, and a hypervariable region H3 (HVR-H3) polypeptide. In some embodiments, an HVR-H1 comprises a polypeptide that is at least about 80% identical to the polypeptide of SEQ ID NO: 94. In some embodiments, an HVR-H1 comprises a polypeptide that is at least about 85% identical to the polypeptide of SEQ ID NO: 94. In some embodiments, an HVR-H1 comprises a polypeptide that is at least about 90% identical to the polypeptide of SEQ ID NO: 94. In some embodiments, an HVR-H1 comprises a polypeptide that is at least about 91%, 92%, 93%, 94%, 95%, 96%, 97%, 98%, or 99% identical to the polypeptide of SEQ ID NO: 94. In some embodiments, an HVR-H1 comprises the polypeptide of SEQ ID NO: 94. In some embodiments, an HVR-H2 comprises a polypeptide that is at least about 80% identical to the polypeptide of SEQ ID NO: 95. In some embodiments, an HVR-H2 comprises a polypeptide that is at least about 85% identical to the polypeptide of SEQ ID NO: 95. In some embodiments, an HVR-H2 comprises a polypeptide that is at least about 90% identical to the polypeptide of SEQ ID NO: 95. In some embodiments, an HVR-H2 comprises a polypeptide that is at least about 91%, 92%, 93%, 94%, 95%, 96%, 97%, 98%, or 99% identical to the polypeptide of SEQ ID NO: 95. In some embodiments, an HVR-H2 comprises the polypeptide of SEQ ID NO: 95. In some embodiments, an HVR-H3 comprises a polypeptide that is at least about 80% identical to the polypeptide of SEQ ID NO: 96. In some embodiments, an HVR-H3 comprises a polypeptide that is at least about 85% identical to the polypeptide of SEQ ID NO: 96. In some embodiments, an HVR-H3 comprises a polypeptide that is at least about 90% identical to the polypeptide of SEQ ID NO: 99. In some embodiments, an HVR-H3 comprises a polypeptide that is at least about 91%, 92%, 93%, 94%, 95%, 96%, 97%, 98%, or 99% identical to the polypeptide of SEQ ID NO: 96. In some embodiments, an HVR-H3 comprises the polypeptide of SEQ ID NO: 96.

An ScFv VL domain may comprise a hypervariable region L1 (HVR-L1) polypeptide, a hypervariable region L2 (HVR-L2) polypeptide, and a hypervariable region L3 (HVR-L3) polypeptide. In some embodiments, an HVR-L1 comprises a polypeptide that is at least about 80% identical to the polypeptide of SEQ ID NO: 98. In some embodiments, an HVR-L1 comprises a polypeptide that is at least about 85% identical to the polypeptide of SEQ ID NO: 98. In some embodiments, an HVR-L1 comprises a polypeptide that is at least about 90% identical to the polypeptide of SEQ ID NO: 98. In some embodiments, an HVR-L1 comprises a polypeptide that is at least about 91%, 92%, 93%, 94%, 95%, 96%, 97%, 98%, or 99% identical to the polypeptide of SEQ ID NO: 98. In some embodiments, an HVR-L1 comprises the polypeptide of SEQ ID NO: 98. In some embodiments, an HVR-L2 comprises a polypeptide that is at least about 80% identical to the polypeptide of SEQ ID NO: 99. In some embodiments, an HVR-L2 comprises a polypeptide that is at least about 85% identical to the polypeptide of SEQ ID NO: 99. In some embodiments, an HVR-L2 comprises a polypeptide that is at least about 90% identical to the polypeptide of SEQ ID NO: 99. In some embodiments, an HVR-L2 comprises a polypeptide that is at least about 91%, 92%, 93%, 94%, 95%, 96%, 97%, 98%, or 99% identical to the polypeptide of SEQ ID NO: 99. In some embodiments, an HVR-L2 comprises the polypeptide of SEQ ID NO: 99. In some embodiments, an HVR-L3 comprises a polypeptide that is at least about 80% identical to the polypeptide of SEQ ID NO: 100. In some embodiments, an HVR-L3 comprises a polypeptide that is at least about 85% identical to the polypeptide of SEQ ID NO: 100. In some embodiments, an HVR-L 3 comprises a polypeptide that is at least about 90% identical to the polypeptide of SEQ ID NO: 100. In some embodiments, an HVR-L3 comprises a polypeptide that is at least about 91%, 92%, 93%, 94%, 95%, 96%, 97%, 98%, or 99% identical to the polypeptide of SEQ ID NO: 100. In some embodiments, an HVR-L3 comprises the polypeptide of SEQ ID NO: 100.

In some embodiments, a CAR comprises an anti-TROP2 VHH polypeptide comprising a hypervariable region H1 (HVR-H1), hypervariable region H2 (HVR-H2), and/or hypervariable region H3 (HVR-H3) chosen from the HVR-H1, HVR-H2, and HVR-H3 polypeptides in Table B.

TABLE B
Anti-TROP2 VHH HVR sequences

| SEQ ID | | |
|---|---|---|
| NO | Notes | Sequences |
| | anti-TROP2, | GX₁X₂X₃X₄X₅X₆X₇ |
| | HVR-H1 | X₁ = F/R/S |
| | polypeptide | X₂ = T/W/S/R |
| | | X₃ = F/L |
| | | X₄ = S/D |
| | | X₅ = Y/H/T/D/W |
| | | X₆ = Y/W |
| | | X₇ = A/W |
| 109 | anti-TROP2, | X₁YADGSX₂T |
| | HVR-H2 | X₁ = I/V |
| | polypeptide | X₂ = A/G/V/L/I |
| 110 | anti-TROP2, | AQGYSDYDRRX₁GX₂YDX₃ |
| | HVR-H3 | X₁ = L/V/I/M/A |
| | polypeptide | X₂ = E/G/A/D/N |
| | | X₃ = Y/F |
| | anti-TROP2, | GX₁X₂X₃DX₄X₅X₆ |
| | HVR-H1 | X₁ = F/T |
| | polypeptide | X₂ = T/W/S/R |
| | | X₃ = L/F |
| | | X₄ = D/Y |
| | | X₅ = Y/W |
| | | X₆ = W/A |
| 112 | anti-TROP2, | X₁SX₂SDX₃X₄T |
| | HVR-H2 | X₁ = I/V |
| | polypeptide | X₂ = S/R |
| | | X₃ = G/S/A |
| | | X₄ = T/S/I |
| | anti-TROP2, | GX₁X₂X₃X₄X₅X₆X₇ |
| | HVR-H1 | X₁ = F/T/R/S |
| | polypeptide | X₂ = T/W/S/R |
| | | X₃ = L/F |
| | | X₄ = D/E |
| | | X₅ = D/Y/H |
| | | X₆ = Y/W |
| | | X₇ = W/A/T |
| 114 | anti-TROP2, | AAX₁SLGYCSRSTPQYX₂YDX₃ |
| | HVR-H3 | X₁ = G/D/A |
| | polypeptide | X₂ = E/G/A/D/N |
| | | X₃ = Y/F |
| 115 | anti-TROP2, | SGSX₁X₂STX₃ |
| | HVR-H2 | X₁ = D/R/S |
| | polypeptide | X₂ = E/G |
| | | X₃ = Y/H/V |
| 116 | anti-TROP2, | AVGX₁X₂X₃X₄SLX₅PYX₆YDY |
| | HVR-H3 | X₁ = S/T |
| | polypeptide | X₂ = T/V |
| | | X₃ = L/W/V |
| | | X₄ = Y/T |
| | | X₅ = A/T |
| | | X₆ = E/G/A/D/N |
| | anti-TROP2, | GX₁X₂X₃X₄X₅X₆X₇ |
| | HVR-H1 | X₁ = F/T/R/S |
| | polypeptide | X₂ = T/W/S/R |
| | | X₃ = L/F/K |
| | | X₄ = D/E |
| | | X₅ = D/Y/H |
| | | X₆ = Y/W |
| | | X₇ = W/A/T |
| 118 | anti-TROP2, | ISX₁SGX₂STN |
| | HVR-H2 | X₁ = S/V |
| | polypeptide | X₂ = S/G |
| | anti-TROP2, | GX₁X₂X₃DX₄X₅X₆ |
| | HVR-H1 | X₁ = F/T/R/S |
| | polypeptide | X₂ = W/T/S/R |
| | | X₃ = L/K |
| | | X₄ = Y/H/T/D/W |
| | | X₅ = Y/W |
| | | X₆ = A/G |
| | anti-TROP2, | GX₁X₂X₃DX₄X₅X₆ |
| | HVR-H1 | X₁ = F/T/R/S |
| | polypeptide | X₂ = T/W/S/R |
| | | X₃ = L/F |
| | | X₄ = Y/H/T/D/W |
| | | X₅ = Y/W |
| | | X₆ = A/G |
| 121 | anti-TROP2, | GX₁X₂FSFYX₃ |
| | HVR-H1 | X₁ = D/F |
| | polypeptide | X₂ = I/A/T |
| | | X₃ = S/V |
| | anti-TROP2, | IX₁SX₂GX₃X₄X₅ |
| | HVR-H2 | X₁ = T/S |
| | polypeptide | X₂ = Y/S |
| | | X₃ = G/S |
| | | X₄ = A/T/S |
| | | X₅ = N/T |
| 123 | anti-TROP2, | X₁ADLDYWVDSMGPY |
| | HVR-H3 | X₁ = N/T |
| | polypeptide | |
| 124 | anti-TROP2, | ITSX₁GX₂X₃X₄ |
| | HVR-H2 | X₁ = Y/G |
| | polypeptide | X₂ = G/S |
| | | X₃ = A/T/S |
| | | X₄ = N/T |
| | anti-TROP2, | GX₁X₂X₃X₄X₅X₆X₇ |
| | HVR-H1 | X₁ = F/R/S |
| | polypeptide | X₂ = T/W/S/R |
| | | X₃ = F/L |
| | | X₄ = S/D |
| | | X₅ = N/Y/H/T/D/W |
| | | X₆ = Y/W |
| | | X₇ = A/W |
| 126 | anti-TROP2, | X₁SX₂FX₃IAI |
| | HVR-H1 | X₁ = S/R |
| | polypeptide | X₂ = I/V |
| | | X₃ = S/N |
| 127 | anti-TROP2, | ISATDVTX₁ |
| | HVR-H2 | X₁ = H/S |
| | polypeptide | |

A CAR described herein may comprise one or more additional components chosen from: (i) one or more signaling domains; (ii) a transmembrane domain; (iii) a hinge; (iv) a cytokine (e.g., IL-15); (v) a T2A sequence; and (vi) one or more linkers. In some embodiments, a CAR comprises a 4-1BB signaling domain. In some embodiments, a CAR comprises a CD3ζ signaling domain. In some embodiments, a CAR comprises a 4-1BB signaling domain and a CD33 signaling domain. In some embodiments, a CAR comprises a CD8a hinge. In some embodiments, an intracellular domain comprises a 4-1BB signaling domain, a CD3Z signaling domain, a T2A sequence, and IL-15. In some embodiments, A CAR comprises, in order (listed in the direction of extracellular domain to intracellular domain), a TROP2 antigen binding domain, a linker, a PD-L1 antigen binding domain, a hinge, a transmembrane domain, a 4-1BB signaling domain, a CD3ζ signaling domain, a T2A sequence, and IL-15. In some embodiments, A CAR comprises, in order (listed in the direction of extracellular domain to intracellular domain), a PD-L1 antigen binding domain, a linker, a TROP2 antigen binding domain, a hinge, a transmembrane domain, a 4-1BB signaling domain, a CD3Z signaling domain, a T2A sequence, and IL-15.

In some embodiments, a CAR comprises one or more cytokines. In some embodiments, a CAR comprises one or more interleukins. For example, a CAR may comprise IL-15 (e.g., an IL-15 comprising the amino acid sequence of SEQ ID NO: 131, or a polypeptide that is at least about 80%, 85%, 90%, 91%, 92%, 93%, 94%, 95%, 96%, 97%, 98%, or 99% identical to the amino acid sequence of SEQ ID NO: 131).

In some embodiments, a CAR comprises a hinge. In some embodiments, a CAR comprises a CD8α hinge. In some embodiments, a CAR comprises a CD8α hinge comprising the polypeptide of SEQ ID NO: 137 or a polypeptide that is at least about 80%, 85%, 90%, 91%, 92%, 93%, 94%, 95%, 96%, 97%, 98%, or 99% identical to the amino acid sequence of SEQ ID NO: 137.

In some embodiments, a CAR comprises a transmembrane domain. In some embodiments, a CAR comprises a CD8α transmembrane domain. In some embodiments, a CAR comprises a CD8α transmembrane domain comprising the polypeptide of SEQ ID NO: 138 or a polypeptide that is at least about 80%, 85%, 90%, 91%, 92%, 93%, 94%, 95%, 96%, 97%, 98%, or 99% identical to the amino acid sequence of SEQ ID NO: 138.

In some embodiments, a CAR comprises one or more signaling domains. In some embodiments, a CAR comprises one or more signaling domains chosen from a 4-1BB signaling domain and a CD3Z signaling domain. In some embodiments, a CAR comprises a 4-1BB signaling domain comprising the polypeptide of SEQ ID NO: 139 or a polypeptide that is at least about 80%, 85%, 90%, 91%, 92%, 93%, 94%, 95%, 96%, 97%, 98%, or 99% identical to the amino acid sequence of SEQ ID NO: 139. In some embodiments, a CAR comprises a CD34 signaling domain comprising the polypeptide of SEQ ID NO: 140 or a polypeptide that is at least about 80%, 85%, 90%, 91%, 92%, 93%, 94%, 95%, 96%, 97%, 98%, or 99% identical to the amino acid sequence of SEQ ID NO: 140.

In some embodiments, a CAR comprises a T2A component. In some embodiments, a CAR comprises a T2A component comprising the polypeptide of SEQ ID NO: 141 or a polypeptide that is at least about 80%, 85%, 90%, 91%, 92%, 93%, 94%, 95%, 96%, 97%, 98%, or 99% identical to the amino acid sequence of SEQ ID NO: 141.

Percent amino acid sequence identity with respect to a reference CAR, VHH, VH, VL, HVR, ScFv, cytokine, or other CAR component polypeptide sequence herein generally refers to the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software.

### Nucleic Acids, Vectors, and Recombinant Methods

Also provided herein are nucleic acid constructs encoding a CAR, and components thereof, described herein. In some embodiments, a nucleic acid comprises a first polynucleotide encoding a first antigen binding domain with binding specificity to TROP2 (e.g., an anti-TROP2 VHH polypeptide; an anti-TROP2 ScFv polypeptide). In some embodiments, a nucleic acid comprises a second polynucleotide encoding a second antigen binding domain with binding specificity to PD-L1 (e.g., an anti-PD-L1 VHH polypeptide; an anti-PD-L1 ScFv polypeptide). In some embodiments, a nucleic acid comprises a first polynucleotide encoding a first antigen binding domain with binding specificity to TROP2 (e.g., an anti-TROP2 VHH polypeptide; an anti-TROP2 ScFv polypeptide), and a second polynucleotide encoding a second antigen binding domain with binding specificity to PD-L1 (e.g., an anti-PD-L1 VHH polypeptide; an anti-PD-L1 ScFv polypeptide). Suitable polynucleotides encoding one or more CAR antigen binding domains described herein may include commercially available polynucleotides, custom synthesized polynucleotides (e.g., from Twist Bioscience, San Francisco, CA), and/or polynucleotides encoding one or more variant polypeptides described herein.

In certain configurations, a CAR nucleic acid construct comprises a polynucleotide encoding an anti-PD-L1 VHH amino acid sequence. In certain configurations, a CAR nucleic acid construct comprises a polynucleotide encoding an anti-PD-L1 VHH amino acid sequence chosen from the VHH amino acid sequences in Table 1 (e.g., SEQ ID NO: 1, SEQ ID NO: 5, SEQ ID NO: 9). In certain configurations, a CAR nucleic acid construct comprises a polynucleotide encoding an amino acid sequence that is at least about 80% identical to an anti-PD-L1 VHH amino acid sequence in

Table 1. In certain configurations, a CAR nucleic acid construct comprises a polynucleotide encoding an amino acid sequence that is at least about 85% identical to an anti-PD-L1 VHH amino acid sequence in Table 1. In certain configurations, a CAR nucleic acid construct comprises a polynucleotide encoding an amino acid sequence that is at least about 90% identical to an anti-PD-L1 VHH amino acid sequence in Table 1. In certain configurations, a CAR nucleic acid construct comprises a polynucleotide encoding an amino acid sequence that is at least about 91%, 92%, 93%, 94%, 95%, 96%, 97%, 98%, or 99% identical to an anti-PD-L1 VHH amino acid sequence in Table 1.

In certain configurations, a CAR nucleic acid construct comprises a polynucleotide encoding an anti-TROP2 VHH amino acid sequence. In certain configurations, a CAR nucleic acid construct comprises a polynucleotide encoding an anti-TROP2 VHH amino acid sequence chosen from the VHH amino acid sequences in Table 2 (e.g., SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, SEQ ID NO: 89). In certain configurations, a CAR nucleic acid construct comprises a polynucleotide encoding an amino acid sequence that is at least about 80% identical to an anti-TROP2 VHH amino acid sequence in Table 2. In certain configurations, a CAR nucleic acid construct comprises a polynucleotide encoding an amino acid sequence that is at least about 85% identical to an anti-TROP2 VHH amino acid sequence in Table 2. In certain configurations, a CAR nucleic acid construct comprises a polynucleotide encoding an amino acid sequence that is at least about 90% identical to an anti-TROP2 VHH amino acid sequence in Table 2. In certain configurations, a CAR nucleic acid construct comprises a polynucleotide encoding an amino acid sequence that is at least about 91%, 92%, 93%, 94%, 95%, 96%, 97%, 98%, or 99% identical to an anti-TROP2 VHH amino acid sequence in Table 2.

In some embodiments, a nucleic acid construct further comprises one or more polynucleotides encoding one or more components chosen from: (i) one or more signaling domains; (ii) a transmembrane domain; (iii) a hinge; (iv) IL-15; (v) a T2A sequence; and (vi) one or more linkers. In some embodiments, a nucleic acid construct comprises a polynucleotide encoding a 4-1BB signaling domain. In some embodiments, a nucleic acid construct comprises a polynucleotide encoding a CD34 signaling domain. In some embodiments, a nucleic acid construct comprises a polynucleotide encoding a 4-1BB signaling domain and a polynucleotide encoding a CD3Z signaling domain. In some embodiments, a nucleic acid construct comprises a polynucleotide encoding a CD8α hinge. Suitable polynucleotides encoding one or more CAR components described herein may include commercially available polynucleotides, custom synthesized polynucleotides (e.g., from Twist Bioscience, San Francisco, CA), and/or polynucleotides encoding one or more variant polypeptides described herein.

For recombinant production of CAR or a component thereof, a nucleic acid encoding the CAR or a component thereof may be isolated and inserted into a replicable vector for further cloning and/or expression. Any suitable vector may be used. Vector components generally include, but are not limited to, one or more of a signal sequence, an origin of replication, one or more marker genes, an enhancer element, a promoter, and a transcription termination sequence.

Methods of genetically modifying a gamma delta T cell to incorporate the nucleic acid encoding a CAR described herein may include any technique known to those skilled in the art. Suitable methodologies include, but are not limited to, viral transduction with lentiviruses, viral transduction with retroviruses, viral transduction with adenoviruses, cellular transfection by electroporation, lipid-based transfection reagents, nanoparticles, calcium chloride-based transfection methods or bacterially-derived transposons, mRNA, nucleic acid, ribonucleoproteins.

In embodiments where lentiviruses/retrovirus/adenovirus can be employed for transduction, inclusion of chemical reagents as would be understood by those in the art to enhance this process can be used. These include for example, but are not limited to, hexadimethrine bromide (Polybrene), recombinant human fibronectin (such as RetroNectin-Takara Clontech), Vectofusin (Miltenyi Biotec), and TRANSPLUS Virus Transduction Enhancer (ALSTEM Cell Advancements). Detecting the efficiency of the transduction and expression of the CAR constructs by the gamma delta T cells may include any technique known to those skilled in the art and may include, but is not limited to, quantitative PCR and antibody-based detection methods such as flow cytometry or western blotting.

### Genome Modifications

In some embodiments, a modified gamma delta T cell comprises one or more genome modifications. A genome modification may include a disruption of a gene (e.g., a gene knock-out (KO)) introduced by way of a suitable genome editing process (e.g., CRISPR-Cas9; CRISPR-Cas12i). In some embodiments, a genome modification is a disruption of a gene in the TGFβ signaling pathway. TGFβ signaling generally controls proliferation, cellular differentiation, and other functions in a variety of cell types, and can play a role in cell cycle control, regulation of the immune system, and development in certain cell types. Disruption of a TGFβ signaling gene may include disruption (e.g., knock out) of any gene encoding a TGFβ signaling pathway protein and/or member of the TGFβ superfamily including TGFβ superfamily type II receptors, type I serine/threonine kinase receptors, type II serine/threonine kinase receptors, TGFβ type I receptor, TGFβ type II receptor, activin receptor, nodal receptor, activin/nodal receptor, activin receptor-like kinases (ALKs; e.g., ALK1, ALK2, ALK3, ALK4, ALK5, ALK6, ALK7 and ALK8); and downstream effectors such as R-SMAD and other SMAD proteins (e.g., SMAD1, SMAD2, SMAD3, SMAD4, SMAD5, SMAD6, SMAD7, SMAD 8/9).

In some embodiments, a modified gamma delta T cell comprises a genome modification in a TGFβ 2 receptor. In some embodiments, a TGFβ 2 receptor gene is knocked out or substantially knocked out in a modified gamma delta T cell. In some embodiments, a population of modified gamma delta T cells comprising a TGFβ 2 receptor knock-out express TGFβ 2 receptors at undetectable levels. In some embodiments, a population of modified gamma delta T cells comprising a TGFβ 2 receptor knock-out express TGFβ 2 receptors at low levels (e.g., 10% or less, 5% or less, 3% or less). Introduction of a genome modification (e.g., a TGFβ 2 receptor knock-out) may be performed in quiescent gamma delta T cells or in activated gamma delta T cells. Introduction of a genome modification (e.g., a TGFβ 2 receptor knock-out) may be performed prior to expansion of gamma delta T cells or after expansion (e.g., by an expansion process described herein).

### Methods of Treatment

Provided herein are methods of treating an infection or cancer in an individual comprising the step of providing said individual with gamma delta T cells (e.g., modified gamma delta T cells). In some embodiments, gamma delta T cells are obtained from a different individual (allogenic treatment). Thus, donor gamma delta T cells are used for the treatment of an infection, for example, of a virus, bacteria, fungi or protozoa, or for treatment of a cancer in a recipient subject where the donor and the recipient are not the same individual. As will be understood, prior to providing the gamma delta T cells to the second subject, these gamma delta T cells are activated and selectively expanded according to a method described herein. In some embodiments, gamma delta T cells are modified as discussed herein, to provide CAR modified gamma delta T cells. In some embodiments, gamma delta T cells are further modified to provide TGFβ receptor knock-out gamma delta T cells.

Also provided is a process for providing gamma delta T cells autologously to a subject comprising the steps of obtaining a sample of gamma delta T cells from a subject and culturing the gamma delta T cells to allow them to be administered back to the subject, where the culturing step comprises expanding and modifying the gamma delta T cells as described herein.

A method of administration to provide gamma delta T cells to a recipient subject may include intravenous, intradermal, intraperitoneal, intrathecal, intratumoral, or subcutaneous injection, for example. Administration may be into an affected area or systemically to the individual. A method of administration can be prophylactic or therapeutic, where a “prophylactically effective amount” or a “therapeutically effective amount” of the gamma delta T cells which is sufficient to show benefit to the individual, is provided. The actual amount administered, and rate and time-course of administration, will depend on the nature and severity of what is being treated. Prescription of treatment, e.g., decisions on dosage etc., is within the responsibility of general practitioners and other medical doctors.

In some embodiments, gamma delta T cells described herein are provided for the treatment of a subject with cancer. In some embodiments, cancer is characterized as a solid tumor-type cancer. In some embodiments, cancer is characterized as an epithelial tissue cancer. In some embodiments, cancer is characterized as squamous tumor-type cancer. In embodiments, cancer can include but is not limited to pancreatic cancer, gastric cancer, renal cell carcinoma, lung cancer (e.g., non-small cell lung cancer), breast cancer, colon cancer, cervical cancer, ovarian cancer, bladder cancer, prostate cancer (e.g., prostate adenocarcinoma), endometrial endometroid carcinoma, oral squamous cell carcinomas, and papillary thyroid carcinoma.

Also provided herein are pharmaceutical compositions comprising modified gamma delta T cells described herein. In some embodiments, a pharmaceutical composition comprises a dose of modified gamma delta T cells suitable to administer to an individual to provide a therapeutic effect. In some embodiments, a pharmaceutical composition further comprises one or more therapeutics chosen from of an antibody immunotherapy, chemotherapeutic agent, biologic, cytokine, or combination thereof.

### Anti-TROP2 Agents and Anti-PD-L1 Agents

Provided herein are agents that bind TROP2 or a portion thereof. Agents that bind TROP2 or a portion thereof may be referred to as anti-TROP2 agents and may include anti-TROP2 nanobodies (e.g., VHH antibodies), anti-TROP2 nanobody fragments (e.g., antigen binding fragments), anti-TROP2 nanobody derivatives, anti-TROP2 antibodies, anti-TROP2 antibody fragments (e.g., antigen binding fragments), and anti-TROP2 antibody derivatives. In some embodiments, the anti-TROP2 agent is isolated (e.g., separated from a component of its natural environment (e.g. an animal, a biological sample)). In some embodiments, the anti-TROP2 agent is non-naturally occurring (e.g., produced by human intervention). In some embodiments, the anti-TROP2 agent is a humanized nanobody, a humanized antibody, or an antigen binding fragment thereof. In some embodiments, the anti-TROP2 is a derivative of a humanized nanobody or humanized antibody. In some embodiments, the anti-TROP2 agent binds TROP2 under laboratory conditions (e.g., binds TROP2 in vitro, binds TROP2 in a flow cytometry assay, binds TROP2 in an ELISA). In some embodiments, the anti-TROP2 agent binds TROP2 under physiological conditions (e.g., binds TROP2 in a cell in a subject).

In some embodiments, an anti-TROP2 agent comprises a VHH polypeptide provided herein or a polypeptide that is 80%, 85%, 90%, 91%, 92%, 93%, 94%, 95%, 96%, 97%, 98%, or 99% identical to a polypeptide provided herein. In some embodiments, an anti-TROP2 agent comprises an HVR-H1 polypeptide provided herein or a polypeptide that is 80%, 85%, 90%, 91%, 92%, 93%, 94%, 95%, 96%, 97%, 98%, or 99% identical to a polypeptide provided herein. In some embodiments, an anti-TROP2 agent comprises an HVR-H2 polypeptide provided herein or a polypeptide that is 80%, 85%, 90%, 91%, 92%, 93%, 94%, 95%, 96%, 97%, 98%, or 99% identical to a polypeptide provided herein. In some embodiments, an anti-TROP2 agent comprises an HVR-H3 polypeptide provided herein or a polypeptide that is 80%, 85%, 90%, 91%, 92%, 93%, 94%, 95%, 96%, 97%, 98%, or 99% identical to a polypeptide provided herein.

Also provided herein are agents that bind PD-L1 or a portion thereof. Agents that bind PD-L1 or a portion thereof may be referred to as anti-PD-L1 agents and may include anti-PD-L1 nanobodies (e.g., VHH antibodies), anti-PD-L1 nanobody fragments (e.g., antigen binding fragments), anti-PD-L1 nanobody derivatives, anti-PD-L1 antibodies, anti-PD-L1 antibody fragments (e.g., antigen binding fragments), and anti-PD-L1 antibody derivatives. In some embodiments, the anti-PD-L1 agent is isolated (e.g., separated from a component of its natural environment (e.g. an animal, a biological sample)). In some embodiments, the anti-PD-L1 agent is non-naturally occurring (e.g., produced by human intervention). In some embodiments, the anti-PD-L1 agent is a humanized nanobody, a humanized antibody, or an antigen binding fragment thereof. In some embodiments, the anti-PD-L1 is a derivative of a humanized nanobody or humanized antibody. In some embodiments, the anti-PD-L1 agent binds PD-L1 under laboratory conditions (e.g., binds PD-L1 in vitro, binds PD-L1 in a flow cytometry assay, binds PD-L1 in an ELISA). In some embodiments, the anti-PD-L1 agent binds PD-L1 under physiological conditions (e.g., binds PD-L1 in a cell in a subject).

In some embodiments, an anti-PD-L1 agent comprises a VHH polypeptide provided herein or a polypeptide that is 80%, 85%, 90%, 91%, 92%, 93%, 94%, 95%, 96%, 97%, 98%, or 99% identical to a polypeptide provided herein. In some embodiments, an anti-PD-L1 agent comprises an HVR-H1 polypeptide provided herein or a polypeptide that is 80%, 85%, 90%, 91%, 92%, 93%, 94%, 95%, 96%, 97%, 98%, or 99% identical to a polypeptide provided herein. In some embodiments, an anti-PD-L1 agent comprises an HVR-H2 polypeptide provided herein or a polypeptide that is 80%, 85%, 90%, 91%, 92%, 93%, 94%, 95%, 96%, 97%, 98%, or 99% identical to a polypeptide provided herein. In some embodiments, an anti-PD-L1 agent comprises an HVR-H3 polypeptide provided herein or a polypeptide that is 80%, 85%, 90%, 91%, 92%, 93%, 94%, 95%, 96%, 97%, 98%, or 99% identical to a polypeptide provided herein.

In some embodiments, an agent herein comprises one or more modifications. For example, immunoconjugates comprising an agent or antibody described herein may be conjugated to a cytotoxic agent such as a toxin (e.g., an enzymatically active toxin of bacterial, fungal, plant or animal origin, or fragments thereof), a radioactive isotope (for example, a radioconjugate), or a cytotoxic drug. Such conjugates are sometimes referred to as antibody-drug conjugates or ADCs. Conjugates can be made using any suitable bifunctional protein coupling agent such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In some embodiments, an agent may be conjugated (e.g., chemically conjugated) to a cell. Such conjugates are sometimes referred to as antibody-cell conjugates (ACC). Agent-cell conjugation may be direct or indirect (e.g., by way of a nucleic acid linker). In some embodiments, an agent may be conjugated to an immune cell (e.g., T cell, NK cell, macrophage, and the like).

In some embodiments, an agent herein comprises one or more detectable markers or labels. For example, for research and diagnostic applications, an agent herein may be labeled with a detectable moiety. Any suitable marker, label, or moiety may be associated with or conjugated to an agent herein. In some embodiments, an agent is labeled with one or more radioisotopes such as, for example, ³⁵S, ¹⁴C, ¹²⁵I, ³H, and ¹³¹I. The agent can be labeled with the radioisotope using techniques known in the art, and radioactivity can be measured using scintillation counting, for example. In some embodiments, an agent is labeled with one or more fluorescent labels such as, for example, rare earth chelates (europium chelates), fluorescein and its derivatives, rhodamine and its derivatives, dansyl, Lissamine, phycoerythrin (PE), Texas Red and Brilliant Violet™ Fluorescent labels may be conjugated to an agent using methods known in the art. Fluorescence can be quantified using a flow cytometer, imaging microscope, or fluorimeter, for example.

### Pharmaceutical Formulations, Dosing, and Routes of Administration

Provided herein are therapeutic compositions comprising an anti-TROP2 agent described herein, and a pharmaceutically acceptable excipient. Also provided herein are therapeutic compositions comprising an anti-PD-L1 agent described herein, and a pharmaceutically acceptable excipient. In some embodiments, an anti-TROP2 agent and/or anti-PD-L1 agent or antigen binding fragment thereof may be formulated in a pharmaceutical composition that is useful for a variety of purposes, including the treatment of diseases or disorders (e.g., cancer). Pharmaceutical compositions comprising one or more antibodies may be administered using a pharmaceutical device to a patient in need thereof, and according to one embodiment of the technology, kits are provided that include such devices. Such devices and kits may be designed for routine administration, including self-administration, of the pharmaceutical compositions herein.

Provided herein are therapeutic compositions comprising an anti-TROP2 agent and/or anti-PD-L1 agent described herein and a pharmaceutically acceptable carrier, excipient, or stabilizer. Therapeutic formulations of an anti-TROP2 agent and/or anti-PD-L1 agent may be prepared for storage by mixing the agent having the desired degree of purity with physiologically and/or pharmaceutically acceptable carriers, excipients, or stabilizers, in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

Formulations herein may also contain more than one active compound as necessary for the particular indication being treated, preferably those with complementary activities that do not adversely affect each other. Such molecules are suitably present in combination in amounts that are effective for the purpose intended. Formulations for in vivo administration generally are sterile. This may be accomplished for instance by filtration through sterile filtration membranes, for example.

Sustained-release preparations may be prepared. Suitable examples of sustained-release preparations include semipermeable matrices of solid hydrophobic polymers containing the agent, which matrices are in the form of shaped articles, e.g., films, or microcapsule. Examples of sustained-release matrices include polyesters, hydrogels (for example, poly(2-hydroxyethyl-methacrylate), or poly(vinyl alcohol)), polylactides, copolymers of L-glutamic acid and gamma ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as such as ethylene-vinyl acetate and lactic acid-glycolic acid enable release of molecules for over 100 days, certain hydrogels release proteins for shorter time periods.

For therapeutic applications, an anti-TROP2 agent and/or anti-PD-L1 agent provided herein may be administered to a mammal, e.g., a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intradermal, intra-articular, intrasynovial, intrathecal, intratumoral, oral, topical, or inhalation routes. For the prevention or treatment of disease, the appropriate dosage of agent will depend on the type of disease to be treated, the severity and course of the disease, whether the agent is administered for preventative or therapeutic purposes, previous therapy, the patient's clinical history and response to the agent, and the discretion of the health professional. The agent may be suitably administered to the patient at one time or over a series of treatments.

In some embodiments, a composition comprising an agent herein can be administered as a monotherapy, and in some embodiments, the composition comprising the agent can be administered as part of a combination therapy. Accordingly, provided herein are therapeutic compositions where the agent is used as an adjuvant or in conjunction with an adjuvant. In some cases, the effectiveness of the agent in preventing or treating diseases may be improved by administering the agent serially or in combination with another drug that is effective for those purposes, such as a chemotherapeutic drug for treatment of cancer or a microbial infection. In other cases, the agent may serve to enhance or sensitize cells to chemotherapeutic treatment, thus permitting efficacy at lower doses and with lower toxicity. Certain combination therapies include, in addition to administration of the composition comprising the agent herein, delivering a second therapeutic regimen selected from the group consisting of a chemotherapeutic agent, radiation therapy, surgery, and a combination of any of the foregoing. Such other agents may be present in the composition being administered or may be administered separately. Also, the agent may be suitably administered serially or in combination with the other agent or modality, e.g., chemotherapeutic drug or radiation for treatment of cancer, infection, and the like, or an immunosuppressive drug.

### Research and Diagnostic

Provided herein are diagnostic reagents comprising an anti-TROP2 agent described herein. For example, anti-TROP2 agents provided herein may be used to detect and/or purify TROP2 from bodily fluid(s) or tissues. Anti-TROP2 agents, for example, may be useful in diagnostic assays for TROP2, e.g., detecting its presence in specific cells, tissues, or bodily fluids. Such diagnostic methods may be useful in diagnosis, e.g., of a hyperproliferative disease or disorder. Also provided herein are methods for detecting TROP2 and/or measuring TROP2 levels in a subject or in a sample from a subject. For example, a method may comprise contacting a sample (e.g., a biological sample known or suspected to contain TROP2) with an agent provided herein, and, if the sample contains TROP2, detecting TROP2: agent complexes. In some embodiments, a TROP2 detection method is performed in vitro. In some embodiments, a TROP2 detection method is performed in vivo. For in vivo diagnostic assays, the agent may be labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintillography.

Also provided herein are reagents comprising an anti-TROP2 agent described herein for non-diagnostic use. Also provided herein are reagents comprising an anti-TROP2 agent described herein for non-therapeutic use. Also provided herein are reagents comprising an anti-TROP2 agent herein for non-diagnostic and non-therapeutic use. For example, provided herein are reagents comprising an anti-TROP2 agent described herein for use in research applications. Research applications may include investigating TROP2 and its role in cell signaling, signal transduction, cell migration, cell growth, development, disease, infection, inflammation, tissue remodeling, tumor growth, tumor angiogenesis, tumor proliferation, tumor migration, tumor invasion, tumor radiation resistance, metastasis, and the like. Also provided herein are methods for detecting TROP2 and/or measuring TROP2 levels in a non-biological sample. For example, a method may comprise contacting a non-biological sample (e.g., a laboratory research sample known or suspected to contain TROP2) with an anti-TROP2 agent provided herein, and, if the sample contains TROP2, detecting TROP2: agent complexes. Laboratory research samples may include non-human animal models, samples from non-human animal models, cell lines, products produced by cell lines, and the like.

Anti-TROP2 agents and antibodies provided herein may be employed in any suitable detection assay, such as flow cytometry, immunohistochemistry, immunofluorescence, mass cytometry, competitive binding assays, direct and indirect sandwich assays, and immunoprecipitation assays.

Also provided herein are diagnostic reagents comprising an anti-PD-L1 agent described herein. For example, anti-PD-L1 agents provided herein may be used to detect and/or purify PD-L1 from bodily fluid(s) or tissues. Anti-PD-L1 agents, for example, may be useful in diagnostic assays for PD-L1, e.g., detecting its presence in specific cells, tissues, or bodily fluids. Such diagnostic methods may be useful in diagnosis, e.g., of a hyperproliferative disease or disorder. Also provided herein are methods for detecting PD-L1 and/or measuring PD-L1 levels in a subject or in a sample from a subject. For example, a method may comprise contacting a sample (e.g., a biological sample known or suspected to contain PD-L1) with an agent provided herein, and, if the sample contains PD-L1, detecting PD-L1: agent complexes. In some embodiments, a PD-L1 detection method is performed in vitro. In some embodiments, a PD-L1 detection method is performed in vivo. For in vivo diagnostic assays, the agent may be labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintillography. Also provided herein are reagents comprising an anti-PD-L1 agent described herein for non-diagnostic use. Also provided herein are reagents comprising an anti-PD-L1 agent described herein for non-therapeutic use. Also provided herein are reagents comprising an anti-PD-L1 agent herein for non-diagnostic and non-therapeutic use. For example, provided herein are reagents comprising an anti-PD-L1 agent described herein for use in research applications. Research applications may include investigating PD-L1 and its role in cell signaling, signal transduction, cell migration, cell growth, development, disease, infection, inflammation, tissue remodeling, tumor growth, tumor angiogenesis, tumor proliferation, tumor migration, tumor invasion, tumor radiation resistance, metastasis, and the like. Also provided herein are methods for detecting PD-L1 and/or measuring PD-L1 levels in a non-biological sample. For example, a method may comprise contacting a non-biological sample (e.g., a laboratory research sample known or suspected to contain PD-L1) with an anti-PD-L1 agent provided herein, and, if the sample contains PD-L1, detecting PD-L1: agent complexes. Laboratory research samples may include non-human animal models, samples from non-human animal models, cell lines, products produced by cell lines, and the like.

Anti-PD-L1 agents and antibodies provided herein may be employed in any suitable detection assay, such as flow cytometry, immunohistochemistry, immunofluorescence, mass cytometry, competitive binding assays, direct and indirect sandwich assays, and immunoprecipitation assays.

Also provided herein are reagents comprising a CAR described herein for non-therapeutic use. For example, provided herein are reagents comprising CAR described herein for use in research applications. Research applications may include investigating PD-L1 and/or TROP2 and their roles in cell signaling, signal transduction, cell migration, cell growth, development, disease, infection, inflammation, tissue remodeling, tumor growth, tumor angiogenesis, tumor proliferation, tumor migration, tumor invasion, tumor radiation resistance, metastasis, and the like.

### Kits

Provided in certain embodiments are kits. The kits may include any components and compositions described herein useful for performing any of the methods described herein, in any suitable combination. Kits may further include any reagents, media, buffers, or other components useful for carrying out any of the methods described herein. For example, a kit may include cell culture media and supplements suitable for culturing T cells (e.g., CTS™ OpTmizer™ T Cell Expansion media (ThermoFisher Scientific) supplemented with ICTSR (e.g., 2.5%), p/s (e.g., 1%), GLUTAMAX (e.g., 2 mM), and human AB serum (e.g., 2.5% at 1 million per mL)). In some embodiments, a kit comprises binding agents comprising (i) a cluster of differentiation 3 (CD3) binding agent (e.g., an antibody or fragment thereof) and (ii) a cluster of differentiation 2 (CD2) binding agent (e.g., an antibody or fragment thereof). In some embodiments, a kit comprises binding agents consisting of a CD3 binding agent and a CD2 binding agent. In some embodiments, a kit comprises one or more cytokines. In some embodiments, a kit comprises one or more cytokines comprising interleukin 15 (IL-15). In some embodiments, a kit comprises one or more cytokines consisting of interleukin 15 (IL-15). In some embodiments, a kit comprises one or more cytokines comprising interleukin 2 (IL-2). In some embodiments, a kit comprises one or more cytokines consisting of interleukin 2 (IL-2).

In some embodiments, a kit comprises cells. In some embodiments, a kit comprises cells derived from a peripheral blood mononuclear cell (PBMC) population. In some embodiments, a kit comprises cells that are depleted of alpha beta T cells.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for selectively expanding gamma delta T cells ex vivo. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### Certain Implementations

Following are non-limiting examples of certain implementations of the technology.
A1. A method for selectively expanding gamma delta T cells ex vivo, comprising:
   expanding the number of gamma delta T cells in an originating cell population under expansion conditions, thereby generating an expanded gamma delta T cell-enriched cell population, wherein the expansion conditions comprise binding agents chosen from two or more of:
   (i) a cluster of differentiation 3 (CD3) binding agent, (ii) a cluster of differentiation 2 (CD2) binding agent, (iii) an NKp46 binding agent, (iv) an NKp44 binding agent, and (v) an NKp30 binding agent.
A2. The method of embodiment A1, wherein the expansion conditions comprise a CD3 binding agent and a CD2 binding agent.
A2.1 The method of embodiment A1, wherein the expansion conditions comprise binding agents consisting of a CD3 binding agent and a CD2 binding agent.
A3. The method of any one of embodiments A1 to A2, wherein the expansion conditions further comprise one or more cytokines.
A4. The method of embodiment A3, wherein the one or more cytokines comprise interleukin 15 (IL-15) and/or interleukin 2 (IL-2).
A5. The method of embodiment A3, wherein the one or more cytokines consist of interleukin 15 (IL-15).
A5.1 The method of embodiment A3, wherein the one or more cytokines consist of interleukin 2 (IL-2).
A6. The method of any one of embodiments A1-A5.1, wherein the originating cell population is derived from a peripheral blood mononuclear cell (PBMC) population.
A7. The method of any one of embodiments A1-A6, wherein the originating cell population is depleted of alpha beta T cells.
A8. The method of any one of embodiments A1-A7, wherein the CD3 binding agent is an antibody or fragment thereof.
A9. The method of any one of embodiments A1-A8, wherein the CD2 binding agent is an antibody or fragment thereof.
A10. The method of any one of embodiments A1-A9, wherein cells are expanded about 200 to 300-fold in the expanded gamma delta T cell-enriched cell population when compared to the originating cell population.
A11. The method of any one of embodiments A1-A10, wherein the expanded gamma delta T cell-enriched cell population comprises multi-subsets of gamma delta T cells.
A12. The method of embodiment A11, wherein the multi-subsets of gamma delta T cells comprise Vdelta1 (Vδ1) T cells and Vdelta2 (Vδ2) T cells.
A13. The method of embodiment A11 or A12, wherein the multi-subsets of gamma delta T cells comprise Vdelta1 (Vδ1) T cells, Vdelta2 (Vδ2) T cells, and non-Vδ1&Vδ2 T cells.
A14. The method of embodiment A12 or A13, wherein the Vδ1 T cells are expanded about 1,000-fold to about 10,000-fold in the expanded gamma delta T cell-enriched cell population when compared to the originating cell population.
A15. The method of any one of embodiments A12-A14, wherein the Vδ2 T cells are expanded about 100-fold to about 1,400-fold in the expanded gamma delta T cell-enriched cell population when compared to the originating cell population.
A15.1 The method of any one of embodiments A12-A14, wherein the Vδ2 T cells are expanded about 350-fold or more in the expanded gamma delta T cell-enriched cell population when compared to the originating cell population.
A16. The method of any one of embodiments A1-A15.1, wherein the expanded gamma delta T cell-enriched cell population comprises about 90% or more gamma delta T cells.
A17. The method of any one of embodiments A1-A16, wherein the expanded gamma delta T cell-enriched cell population comprises about 95% or more gamma delta T cells.
A18. The method of any one of embodiments A1-A17, wherein the expanded gamma delta T cell-enriched cell population comprises between about 10-55% Vδ1 T cells, between about 30-80% Vδ2 T cells, and between about 5-15% non-Vδ1&Vδ2 T cells.
A18.1 The method of any one of embodiments A1-A17, wherein the expanded gamma delta T cell-enriched cell population comprises between about 50-55% Vδ1 T cells, between about 28-33% Vδ2 T cells, and between about 6-11% non-Vδ1&Vδ2 T cells.
A19. The method of any one of embodiments A1-A18.1, wherein the expanded gamma delta T cell-enriched cell population comprises about 5% natural killer (NK) cells or less.
A20. The method of any one of embodiments A1-A19, wherein the expanded gamma delta T cell-enriched cell population comprises about 3% natural killer (NK) cells or less.
A21. The method of any one of embodiments A1-A20, wherein the expanded gamma delta T cell-enriched cell population comprises about 5% alpha beta T cells or less.
A22. The method of any one of embodiments A1-A21, wherein the expanded gamma delta T cell-enriched cell population comprises about 3% alpha beta T cells or less.
A22.1 The method of any one of embodiments A1-A22, wherein the expanded gamma delta T cell-enriched cell population comprises about 0.5% alpha beta T cells or less.
A22.2 The method of any one of embodiments A1-A22.1, wherein the expanded gamma delta T cell-enriched cell population comprises about 0.1% alpha beta T cells or less.
A23. The method of any one of embodiments A1-A22.2, further comprising isolating the gamma delta T cells from the expanded gamma delta T cell-enriched cell population.
A24. The method of any one of embodiments A1-A23, further comprising storing the expanded gamma delta T cell-enriched cell population or isolate thereof in a cell bank.
A25. The method of any one of embodiments A1-A24, further comprising, prior to the expanding, transducing or transfecting the gamma delta T cells with a nucleic acid of any one of embodiments D1-D18 or a vector of any one of embodiments D21-D23.
A26. The method of any one of embodiments A1-A25, further comprising introducing a genome modification into cells in the expanded gamma delta T cell-enriched cell population by a genome editing process.
A27. The method of embodiment A26, wherein the genome modification is a disrupted gene encoding TGF-beta receptor 2.
A28. An expanded gamma delta T cell-enriched cell population produced by the method of any one of embodiments A1-A27.
A29. Use of the expanded gamma delta T cell-enriched cell population of embodiment A28 for production of genetically modified cells.
A30. Use of the expanded gamma delta T cell-enriched cell population of embodiment A28 or A29 for treatment of a subject in need thereof.
A31. The use of embodiment A30, wherein the subject has cancer.
A32. The use of embodiment A30 or A31, wherein the subject has solid tumor cancer.
A33. The use of any one of embodiments A30-A32, wherein the subject has one or more of pancreatic cancer, gastric cancer, renal cell carcinoma, lung cancer, breast cancer, colon cancer, cervical cancer, ovarian cancer, bladder cancer, prostate cancer, endometrial endometroid carcinoma, oral squamous cell carcinomas, and papillary thyroid carcinoma.
B1. A kit for selectively expanding gamma delta T cells ex vivo comprising binding agents chosen from two or more of: (i) a cluster of differentiation 3 (CD3) binding agent, (ii) a cluster of differentiation 2 (CD2) binding agent, (iii) an NKp46 binding agent, (iv) an NKp44 binding agent, and (v) an NKp30 binding agent.
B2. The kit of embodiment B1, wherein the binding agents comprise a CD3 binding agent and a CD2 binding agent.
B2.1 The kit of embodiment B1, wherein the binding agents consist of a CD3 binding agent and a CD2 binding agent.
B3. The kit of any one of embodiments B1 to B2, further comprising one or more cytokines.
B4. The kit of embodiment B3, wherein the one or more cytokines comprise interleukin 15 (IL-15) and/or interleukin 2 (IL-2).
B5. The kit of embodiment B3, wherein the one or more cytokines consist of interleukin 15 (IL-15).
B5.1 The kit of embodiment B3, wherein the one or more cytokines consist of interleukin 2 (IL-2).
B6. The kit of any one of embodiments B1-B5.1, further comprising cells derived from a peripheral blood mononuclear cell (PBMC) population.
B7. The kit of embodiment B6, wherein the cells are depleted of alpha beta T cells.
B8. The kit of any one of embodiments B1-B7, wherein the CD3 binding agent is an antibody or fragment thereof.
B9. The kit of any one of embodiments B1-B8, wherein the CD2 binding agent is an antibody or fragment thereof.
C1. A modified gamma delta T cell comprising a chimeric antigen receptor (CAR), wherein the CAR comprises a first antigen binding domain with binding specificity to TROP2 and a second antigen binding domain with binding specificity to PD-L1.
C1.1 The modified gamma delta T cell of embodiment C1, wherein the first antigen binding domain specifically binds to a target in TROP2.
C1.2 The modified gamma delta T cell of embodiment C1, wherein the first antigen binding domain specifically binds to two or more targets in TROP2.
C1.3 The modified gamma delta T cell of any one of embodiments C1-C1.2, wherein the second antigen binding domain specifically binds to a target in PD-L1.
C1.4 The modified gamma delta T cell of any one of embodiments C1-C1.2, wherein the second antigen binding domain specifically binds to two or more targets in PD-L1.
C2. The modified gamma delta T cell of any one of embodiments C1-C1.4, wherein the first antigen binding domain comprises an amino acid sequence that is about 90% or more identical to an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
C3. The modified gamma delta T cell of any one of embodiments C1-C1.4, wherein the first antigen binding domain comprises an amino acid sequence that is about 95% or more identical to an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
C4. The modified gamma delta T cell of any one of embodiments C1-C1.4, wherein the first antigen binding domain comprises an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
C5. The modified gamma delta T cell of any one of embodiments C1-C4, wherein the first antigen binding domain comprises:
   a) a hypervariable region H1 (HVR-H1) polypeptide chosen from SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, and SEQ ID NO: 90;
   b) a hypervariable region H2 (HVR-H2) polypeptide chosen from SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, and SEQ ID NO: 91; and
   c) a hypervariable region H3 (HVR-H3) polypeptide chosen from SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, and SEQ ID NO: 92.
C6. The modified gamma delta T cell of any one of embodiments C1-C5, wherein the second antigen binding domain comprises an amino acid sequence that is about 90% or more identical to an amino acid sequence chosen from SEQ ID NO: 1, SEQ ID NO: 5, and SEQ ID NO: 9.
C7. The modified gamma delta T cell of any one of embodiments C1-C5, wherein the second antigen binding domain comprises an amino acid sequence that is about 95% or more identical to an amino acid sequence chosen from SEQ ID NO: 1, SEQ ID NO: 5, and SEQ ID NO: 9.
C8. The modified gamma delta T cell of any one of embodiments C1-C5, wherein the second antigen binding domain comprises an amino acid sequence chosen from SEQ ID NO: 1, SEQ ID NO: 5, and SEQ ID NO: 9.
C9. The modified gamma delta T cell of any one of embodiments C1-C8, wherein the second antigen binding domain comprises:
   a) a hypervariable region H1 (HVR-H1) polypeptide chosen from SEQ ID NO: 2, SEQ ID NO: 6, and SEQ ID NO: 10;
   b) a hypervariable region H2 (HVR-H2) polypeptide chosen from SEQ ID NO: 3, SEQ ID NO: 7, and SEQ ID NO: 11; and
   c) a hypervariable region H3 (HVR-H3) polypeptide chosen from SEQ ID NO: 4, SEQ ID NO: 8, and SEQ ID NO: 12.
C10. The modified gamma delta T cell of any one of embodiments C1-C9, wherein the CAR comprises a polypeptide that is about 90% or more identical to a polypeptide chosen from SEQ ID NO: 102, SEQ ID NO: 103, SEQ ID NO: 104, SEQ ID NO: 105, SEQ ID NO: 106, and SEQ ID NO: 107.
C11. The modified gamma delta T cell of any one of embodiments C1-C9, wherein the CAR comprises a polypeptide that is about 95% or more identical to a polypeptide chosen from SEQ ID NO: 102, SEQ ID NO: 103, SEQ ID NO: 104, SEQ ID NO: 105, SEQ ID NO: 106, and SEQ ID NO: 107.
C12. The modified gamma delta T cell of any one of embodiments C1-C9, wherein the CAR comprises a polypeptide chosen from SEQ ID NO: 102, SEQ ID NO: 103, SEQ ID NO: 104, SEQ ID NO: 105, SEQ ID NO: 106, and SEQ ID NO: 107.
C13. The modified gamma delta T cell of any one of embodiments C1-C12, wherein the CAR further comprises one or more components chosen from: (i) one or more signaling domains; (ii) a transmembrane domain; (iii) a hinge; (iv) IL-15; (v) a T2A sequence; and (vi) a linker.
C14. The modified gamma delta T cell of embodiment C13, wherein the one or more signaling domains comprise 4-1BB signaling domain, CD3ζ signaling domain, or 4-1BB signaling domain and CD33 signaling domain.
C15. The modified gamma delta T cell of embodiment C13 or C14, wherein the hinge comprises a CD8α hinge.
C16. The modified gamma delta T cell of any one of embodiments C1-C15, further comprising a genome modification.
C17. The modified gamma delta T cell of embodiment C16, wherein the genome modification is a disrupted gene encoding TGF-beta receptor 2.
C18. The modified gamma delta T cell of any one of embodiments C1-C17, for use in treatment of a subject in need thereof.
C19. The modified gamma delta T cell of embodiment C18, wherein the subject has cancer.
C20. The modified gamma delta T cell of embodiment C18 or C19, wherein the subject has solid tumor cancer.
C21. The modified gamma delta T cell of any one of embodiments C18 to C20, wherein the subject has one or more of pancreatic cancer, gastric cancer, renal cell carcinoma, lung cancer, breast cancer, colon cancer, cervical cancer, ovarian cancer, bladder cancer, prostate cancer, endometrial endometroid carcinoma, oral squamous cell carcinomas, and papillary thyroid carcinoma.
C22. The modified gamma delta T cell of any one of embodiments C1-C21, wherein the first antigen binding domain comprises a single-chain variable fragment (ScFv).
C23. The modified gamma delta T cell of embodiment C22, wherein the ScFv comprises a variable heavy chain (VH) domain and a variable light chain (VL) domain, wherein the VH domain comprises a hypervariable region H1 (HVR-H1) polypeptide, a hypervariable region H2 (HVR-H2) polypeptide, and a hypervariable region H3 (HVR-H3) polypeptide, wherein the HVR-H1 comprises the polypeptide of SEQ ID NO: 94, the HVR-H2 comprises the polypeptide of SEQ ID NO: 95, and the HVR-H3 comprises the polypeptide of SEQ ID NO: 96; and the VL domain comprises a hypervariable region L1 (HVR-L1) polypeptide, a hypervariable region L2 (HVR-L2) polypeptide, and a hypervariable region L3 (HVR-L3) polypeptide, wherein the HVR-L1 comprises the polypeptide of SEQ ID NO: 98, the HVR-L2 comprises the polypeptide of SEQ ID NO: 99, and the HVR-L3 comprises the polypeptide of SEQ ID NO: 100.
C24. The modified gamma delta T cell of embodiment C23, wherein the VH domain comprises the polypeptide of SEQ ID NO: 93 and the VL domain comprises the polypeptide of SEQ ID NO: 97.
D1. A nucleic acid encoding a chimeric antigen receptor (CAR), wherein the nucleic acid comprises (i) a first polynucleotide encoding a first antigen binding domain with binding specificity to TROP2, and (ii) a second polynucleotide encoding a second antigen binding domain with binding specificity to PD-L1.
D1.1 The nucleic acid of embodiment D1, wherein the first antigen binding domain specifically binds to a target in TROP2.
D1.2 The nucleic acid of embodiment D1, wherein the first antigen binding domain specifically binds to two or more targets in TROP2.
D1.3 The nucleic acid of any one of embodiments D1-D1.2, wherein the second antigen binding domain specifically binds to a target in PD-L1.
D1.4 The nucleic acid of any one of embodiments D1-D1.2, wherein the second antigen binding domain specifically binds to two or more targets in PD-L1.
D2. The nucleic acid of any one of embodiments D1-D1.4, wherein the first polynucleotide encodes a first antigen binding domain comprising an amino acid sequence that is about 90% or more identical to an amino acid sequence chosen from SEQ ID NOs: SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
D3. The nucleic acid of any one of embodiments D1-D1.4, wherein the first polynucleotide encodes a first antigen binding domain comprising an amino acid sequence that is about 95% or more identical to an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
D4. The nucleic acid of any one of embodiments D1-D1.4, wherein the first polynucleotide encodes a first antigen binding domain comprising an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
D5. The nucleic acid of any one of embodiments D1-D4, wherein the first polynucleotide encodes a first antigen binding domain comprising:
   a) a hypervariable region H1 (HVR-H1) polypeptide chosen from SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, and SEQ ID NO: 90;
   b) a hypervariable region H2 (HVR-H2) polypeptide chosen from SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, and SEQ ID NO: 91; and
   c) a hypervariable region H3 (HVR-H3) polypeptide chosen from SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, and SEQ ID NO: 92.
D6. The nucleic acid of any one of embodiments D1-D5, wherein the second polynucleotide encodes a second antigen binding domain comprising an amino acid sequence that is about 90% or more identical to an amino acid sequence chosen from SEQ ID NO: 1, SEQ ID NO: 5, and SEQ ID NO: 9.
D7. The nucleic acid of any one of embodiments D1-D5, wherein the second polynucleotide encodes a second antigen binding domain comprising an amino acid sequence that is about 95% or more identical to an amino acid sequence chosen from SEQ ID NO: 1, SEQ ID NO: 5, and SEQ ID NO: 9.
D8. The nucleic acid of any one of embodiments D1-D5, wherein the second polynucleotide encodes a second antigen binding domain comprising an amino acid sequence chosen from SEQ ID NO: 1, SEQ ID NO: 5, and SEQ ID NO: 9.
D9. The nucleic acid of any one of embodiments D1-D8, wherein the second polynucleotide encodes a second antigen binding domain comprising:
   a) a hypervariable region H1 (HVR-H1) polypeptide chosen from SEQ ID NO: 2, SEQ ID NO: 6, and SEQ ID NO: 10;
   b) a hypervariable region H2 (HVR-H2) polypeptide chosen from SEQ ID NO: 3, SEQ ID NO: 7, and SEQ ID NO: 11; and
   c) a hypervariable region H3 (HVR-H3) polypeptide chosen from SEQ ID NO: 4, SEQ ID NO: 8, and SEQ ID NO: 12.
D10. The nucleic acid of any one of embodiments D1-D9, wherein the CAR comprises a polypeptide that is about 90% or more identical to a polypeptide chosen from SEQ ID NO: 102, SEQ ID NO: 103, SEQ ID NO: 104, SEQ ID NO: 105, SEQ ID NO: 106, and SEQ ID NO: 107.
D11. The nucleic acid of any one of embodiments D1-D9, wherein the CAR comprises a polypeptide that is about 95% or more identical to a polypeptide chosen from SEQ ID NO: 102, SEQ ID NO: 103, SEQ ID NO: 104, SEQ ID NO: 105, SEQ ID NO: 106, and SEQ ID NO: 107.
D12. The nucleic acid of any one of embodiments D1-D9, wherein the CAR comprises a polypeptide chosen from SEQ ID NO: 102, SEQ ID NO: 103, SEQ ID NO: 104, SEQ ID NO: 105, SEQ ID NO: 106, and SEQ ID NO: 107.
D13. The nucleic acid of any one of embodiments D1-D12, wherein the nucleic acid further comprises one or more polynucleotides encoding one or more components chosen from: (i) one or more signaling domains; (ii) a transmembrane domain; (iii) a hinge; (iv) IL-15; (v) a T2A sequence; and (vi) a linker.
D14. The nucleic acid of embodiment D13, wherein the one or more signaling domains comprise 4-1BB signaling domain, CD3Z signaling domain, or 4-1BB signaling domain and CD33 signaling domain.
D15. The nucleic acid of embodiment D13 or D14, wherein the hinge comprises a CD8α hinge.
D16. The nucleic acid of any one of embodiments D1-D15, wherein the first polynucleotide encodes a first antigen binding domain comprising a single-chain variable fragment (ScFv).
D17. The nucleic acid of embodiment D16, wherein the ScFv comprises a variable heavy chain (VH) domain and a variable light chain (VL) domain, wherein the VH domain comprises a hypervariable region H1 (HVR-H1) polypeptide, a hypervariable region H2 (HVR-H2) polypeptide, and a hypervariable region H3 (HVR-H3) polypeptide, wherein the HVR-H1 comprises the polypeptide of SEQ ID NO: 94, the HVR-H2 comprises the polypeptide of SEQ ID NO: 95, and the HVR-H3 comprises the polypeptide of SEQ ID NO: 96; and the VL domain comprises a hypervariable region L1 (HVR-L1) polypeptide, a hypervariable region L2 (HVR-L2) polypeptide, and a hypervariable region L3 (HVR-L3) polypeptide, wherein the HVR-L1 comprises the polypeptide of SEQ ID NO: 98, the HVR-L2 comprises the polypeptide of SEQ ID NO: 99, and the HVR-L3 comprises the polypeptide of SEQ ID NO: 100.
D18. nucleic acid of embodiment D17, wherein the VH domain comprises the polypeptide of SEQ ID NO: 93 and the VL domain comprises the polypeptide of SEQ ID NO: 97.
D19. A modified cell transfected or transduced with a nucleic acid of any one of embodiments D1-D19.
D20. The modified cell of embodiment D19, wherein the modified cell is a gamma delta T cell. D21. A recombinant vector comprising the nucleic acid of any one of embodiments D1-D18.
D22. The recombinant vector of embodiment D21, wherein the vector is a viral vector.
D23. The recombinant vector of embodiment D21, wherein the vector is a non-viral vector.
E1. A chimeric antigen receptor (CAR) comprising a first antigen binding domain with binding specificity to TROP2 a second antigen binding domain with binding specificity to PD-L1.
E1.1 The CAR of embodiment E1, wherein the first antigen binding domain specifically binds to a target in TROP2.
E1.2 The CAR of embodiment E1, wherein the first antigen binding domain specifically binds to two or more targets in TROP2.
E1.3 The CAR of any one of embodiments E1-E1.2, wherein the second antigen binding domain specifically binds to a target in PD-L1.
E1.4 The CAR of any one of embodiments E1-E1.2, wherein the second antigen binding domain specifically binds to two or more targets in PD-L1.
E2. The CAR of any one of embodiments E1-E1.4, wherein the first antigen binding domain comprises an amino acid sequence that is about 90% or more identical to an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
E3. The CAR of any one of embodiments E1-E1.4, wherein the first antigen binding domain comprises an amino acid sequence that is about 95% or more identical to an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
E4. The CAR of any one of embodiments E1-E1.4, wherein the first antigen binding domain comprises an amino acid sequence chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
E5. The CAR of any one of embodiments E1-E4, wherein the first antigen binding domain comprises:
   a) a hypervariable region H1 (HVR-H1) polypeptide chosen from SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, and SEQ ID NO: 90;
   b) a hypervariable region H2 (HVR-H2) polypeptide chosen from SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, and SEQ ID NO: 91; and
   c) a hypervariable region H3 (HVR-H3) polypeptide chosen from SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, and SEQ ID NO: 92.
E6. The CAR of any one of embodiments E1-E5, wherein the second antigen binding domain comprises an amino acid sequence that is about 90% or more identical to an amino acid sequence chosen from SEQ ID NO: 1, SEQ ID NO: 5, and SEQ ID NO: 9.
E7. The CAR of any one of embodiments E1-E5, wherein the second antigen binding domain comprises an amino acid sequence that is about 95% or more identical to an amino acid sequence chosen from SEQ ID NO: 1, SEQ ID NO: 5, and SEQ ID NO: 9.
E8. The CAR of any one of embodiments E1-E5, wherein the second antigen binding domain comprises an amino acid sequence chosen from SEQ ID NO: 1, SEQ ID NO: 5, and SEQ ID NO: 9.
E9. The CAR of any one of embodiments E1-E8, wherein the second antigen binding domain comprises:
   a) a hypervariable region H1 (HVR-H1) polypeptide chosen from SEQ ID NO: 2, SEQ ID NO: 6, and SEQ ID NO: 10;
   b) a hypervariable region H2 (HVR-H2) polypeptide chosen from SEQ ID NO: 3, SEQ ID NO: 7, and SEQ ID NO: 11; and
   c) a hypervariable region H3 (HVR-H3) polypeptide chosen from SEQ ID NO: 4, SEQ ID NO: 8, and SEQ ID NO: 12.
E10. The CAR of any one of embodiments E1-E9, comprising a polypeptide that is about 90% or more identical to a polypeptide chosen from SEQ ID NO: 102, SEQ ID NO: 103, SEQ ID NO: 104, SEQ ID NO: 105, SEQ ID NO: 106, and SEQ ID NO: 107.
E11. The CAR of any one of embodiments E1-E9, comprising a polypeptide that is about 95% or more identical to a polypeptide chosen from SEQ ID NO: 102, SEQ ID NO: 103, SEQ ID NO: 104, SEQ ID NO: 105, SEQ ID NO: 106, and SEQ ID NO: 107.
E12. The CAR of any one of embodiments E1-E9, comprising a polypeptide chosen from SEQ ID NO: 102, SEQ ID NO: 103, SEQ ID NO: 104, SEQ ID NO: 105, SEQ ID NO: 106, and SEQ ID NO: 107.
E13. The CAR of any one of embodiments E1-E12, wherein the nucleic acid further comprises one or more polynucleotides encoding one or more components chosen from: (i) one or more signaling domains; (ii) a transmembrane domain; (iii) a hinge; (iv) IL-15; (v) a T2A sequence; and (vi) a linker.
E14. The CAR of embodiment E13, wherein the one or more signaling domains comprise 4-1BB signaling domain, CD3Z signaling domain, or 4-1BB signaling domain and CD34 signaling domain.
E15. The CAR of embodiment E13 or E14, wherein the hinge comprises a CD8α hinge.
E16. The CAR of any one of embodiments E1-E15, wherein the wherein the first antigen binding domain comprises a single-chain variable fragment (ScFv).
E17. The CAR of embodiment E16, wherein the ScFv comprises a variable heavy chain (VH) domain and a variable light chain (VL) domain, wherein the VH domain comprises a hypervariable region H1 (HVR-H1) polypeptide, a hypervariable region H2 (HVR-H2) polypeptide, and a hypervariable region H3 (HVR-H3) polypeptide, wherein the HVR-H1 comprises the polypeptide of SEQ ID NO: 94, the HVR-H2 comprises the polypeptide of SEQ ID NO: 95, and the HVR-H3 comprises the polypeptide of SEQ ID NO: 96; and the VL domain comprises a hypervariable region L1 (HVR-L1) polypeptide, a hypervariable region L2 (HVR-L2) polypeptide, and a hypervariable region L3 (HVR-L3) polypeptide, wherein the HVR-L1 comprises the polypeptide of SEQ ID NO: 98, the HVR-L2 comprises the polypeptide of SEQ ID NO: 99, and the HVR-L3 comprises the polypeptide of SEQ ID NO: 100.
E18. The CAR of embodiment E17, wherein the VH domain comprises the polypeptide of SEQ ID NO: 93 and the VL domain comprises the polypeptide of SEQ ID NO: 97.
E19. A modified cell comprising the CAR of any one of embodiments E1-E18.
E20. The modified cell of embodiment E19, wherein the modified cell is a gamma delta T cell.
F1. A pharmaceutical composition comprising the modified gamma delta T cell of any one of embodiments C1-C24.
F2. A pharmaceutical composition of embodiment F1, further comprising one or more therapeutics chosen from an antibody immunotherapy, chemotherapeutic agent, biologic, cytokine, or combination thereof.
G1. A method of treatment comprising administering a therapeutic dose of the modified gamma delta T cell of any one of embodiments C1-C24 to a subject in need thereof.
G2. The method of treatment of embodiment G1, further comprising co-administering one or more therapeutics chosen from an antibody immunotherapy, chemotherapeutic agent, biologic, cytokine, or combination thereof.
G3. The method of treatment of embodiment G1 or G2, wherein the subject has cancer.
G4. The method of treatment of any one of embodiments G1-G3, wherein the subject has solid tumor cancer.
G5. The method of treatment of any one of embodiments G1-G4, wherein the subject has one or more of pancreatic cancer, gastric cancer, renal cell carcinoma, lung cancer, breast cancer, colon cancer, cervical cancer, ovarian cancer, bladder cancer, prostate cancer, endometrial endometroid carcinoma, oral squamous cell carcinomas, and papillary thyroid carcinoma.
H1. An agent that binds TROP2, or a fragment thereof, comprising:
   a) a hypervariable region H1 (HVR-H1) polypeptide chosen from SEQ ID NO: 14, SEQ ID NO: 18, SEQ ID NO: 22, SEQ ID NO: 26, SEQ ID NO: 30, SEQ ID NO: 34, SEQ ID NO: 38, SEQ ID NO: 42, SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74, SEQ ID NO: 78, SEQ ID NO: 82, SEQ ID NO: 86, and SEQ ID NO: 90;
   b) a hypervariable region H2 (HVR-H2) polypeptide chosen from SEQ ID NO: 15, SEQ ID NO: 19, SEQ ID NO: 23, SEQ ID NO: 27, SEQ ID NO: 31, SEQ ID NO: 35, SEQ ID NO: 39, SEQ ID NO: 43, SEQ ID NO: 47, SEQ ID NO: 51, SEQ ID NO: 55, SEQ ID NO: 59, SEQ ID NO: 63, SEQ ID NO: 67, SEQ ID NO: 71, SEQ ID NO: 75, SEQ ID NO: 79, SEQ ID NO: 83, SEQ ID NO: 87, and SEQ ID NO: 91; and
   c) a hypervariable region H3 (HVR-H3) polypeptide chosen from SEQ ID NO: 16, SEQ ID NO: 20, SEQ ID NO: 24, SEQ ID NO: 28, SEQ ID NO: 32, SEQ ID NO: 36, SEQ ID NO: 40, SEQ ID NO: 44, SEQ ID NO: 48, SEQ ID NO: 52, SEQ ID NO: 56, SEQ ID NO: 60, SEQ ID NO: 64, SEQ ID NO: 68, SEQ ID NO: 72, SEQ ID NO: 76, SEQ ID NO: 80, SEQ ID NO: 83, SEQ ID NO: 88, and SEQ ID NO: 92.
H2. The agent of embodiment H1, comprising a polypeptide that is about 90% or more identical to a polypeptide chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
H3. The agent of embodiment H1, comprising a polypeptide that is about 95% or more identical to a polypeptide chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
H4. The agent of embodiment H1, comprising a polypeptide chosen from SEQ ID NO: 13, SEQ ID NO: 17, SEQ ID NO: 21, SEQ ID NO: 25, SEQ ID NO: 29, SEQ ID NO: 33, SEQ ID NO: 37, SEQ ID NO: 41, SEQ ID NO: 45, SEQ ID NO: 49, SEQ ID NO: 53, SEQ ID NO: 57, SEQ ID NO: 61, SEQ ID NO: 65, SEQ ID NO: 69, SEQ ID NO: 73, SEQ ID NO: 77, SEQ ID NO: 81, SEQ ID NO: 85, and SEQ ID NO: 89.
H5. The agent of any one of embodiments H1-H4, wherein the agent is an antibody.
H6. The agent of any one of embodiments H1-H4, wherein the agent is a nanobody.
H7. The agent of any one of embodiments H1-H4, wherein the agent is a VHH.
I1. An agent that binds PD-L1, or a fragment thereof, comprising:
   a) a hypervariable region H1 (HVR-H1) polypeptide chosen from SEQ ID NO: 2, SEQ ID NO: 6, and SEQ ID NO: 10;
   b) a hypervariable region H2 (HVR-H2) polypeptide chosen from SEQ ID NO: 3, SEQ ID NO: 7, and SEQ ID NO: 11; and
   c) a hypervariable region H3 (HVR-H3) polypeptide chosen from SEQ ID NO: 4, SEQ ID NO: 8, and SEQ ID NO: 12.
I2. The agent of embodiment 11, comprising a polypeptide that is about 90% or more identical to a polypeptide chosen from SEQ ID NO: 1, SEQ ID NO: 5, and SEQ ID NO: 9.
I3. The agent of embodiment 11, comprising a polypeptide that is about 95% or more identical to a polypeptide chosen from SEQ ID NO: 1, SEQ ID NO: 5, and SEQ ID NO: 9.
I4. The agent of embodiment 11, comprising a polypeptide chosen from SEQ ID NO: 1, SEQ ID NO: 5, and SEQ ID NO: 9.
I5. The agent of any one of embodiments 11-14, wherein the agent is an antibody.
I6. The agent of any one of embodiments 11-14, wherein the agent is a nanobody.
I7. The agent of any one of embodiments 11-14, wherein the agent is a VHH.

### EXAMPLES

The examples set forth below illustrate certain implementations and do not limit the technology.

### Example 1: Production of Modified Multi-Subsets of Gamma Delta T Cells

This Example describes a process for generating highly activated and expanded multi-subsets of gamma delta (γδ) T cells. Typically, γδ T cell therapy products are limited to one γδ subtype: either Vδ1 T cells or Vδ2 T cells, due to production limitations, e.g., the use of Vδ1 TCR activating Abs, or phosphoantigens such as Zoledronate or BTN3A1/BTN2A1 agonists that activate only Vδ2 TCR. Given the diverse and complementary anti-tumor activities of both Vδ1 and Vδ2 T cells, a process described herein was developed that effectively activates and expands both Vδ1 and Vδ2 T cells along with non-Vδ1&Vδ2 T cells.

In this Example, a process for generating the modified γδ T cells provided herein is described. In brief, the process starts with the collection of human apheresis products from healthy donors, followed by αβ T cell depletion using CliniMACS® TCRα/β Product Line (Miltenyi) and cryopreservation.

For research scale production, a frozen aliquot of 1×10⁶ αβ T-depleted leukapheresis donor sample was thawed and suspended in CTS™ OpTmizer™ T Cell Expansion media (ThermoFisher Scientific) supplemented with 2.5% ICTSR, 1% p/s, 2 mM GLUTAMAX and 2.5% human AB serum at 1 million cells per mL. Soluble anti-CD3 (OKT3) and anti-CD2 agonist (Miltenyi) and human interleukin 15 (IL-15) (R&D) were added at the beginning of cell culture. In certain workflow variations, human interleukin 2 (IL-2) may be used in addition to or instead of IL-15 at the beginning of cell culture. In certain workflow variations, soluble anti-CD3 (OKT3) combined with one or more of NKp44 ligand (PDGF-DD), anti-NKp46, and anti-NKp30; and human interleukin 15 (IL-15) (R&D) and/or human interleukin 2 (IL-2) may be added. Cells continued in culture and were fed with IL-15 and fresh medium at regular intervals. In certain workflow variations, cells may be fed with IL-2 and fresh medium at regular intervals until cell harvest. The process was continuously monitored to determine viability, kinetics of expansion and phenotype. Following 14 to 21 days of culture, expanded γδ T cells were phenotyped and cytotoxicity was determined using a coculture assay with multiple tumor cell lines.

For CAR γδ T cell generation, γδ T cells were transduced with retroviral vector encoding TROP2 CAR-IL-15, or PD-L1 CAR-IL-15 or TROP2-PD-L1 CAR-IL-15 on Day 4 (or Day 3-6) in culture. CTS complete medium containing CAR retroviral vector supernatant at a MOI=5 and 20 μg/mL of Vectofusin-1 (Miltenyi) were mixed with cell suspension at 1 to 1 ratio and incubated at 37° C., 5% CO2 overnight. On the following day, 4× volumes of prewarmed CST complete medium containing human IL-2 300 IU/mL was added to continue the cell culture. To knockout TGFβR2 gene in γδ T cells, CRISPR/Cas9 system was delivered as a ribonucleoprotein (RNP) complex comprising Cas9 protein and a single guide RNA targeting TGFβR2 by electroporation on MaxCyte ATX (MaxCyte) on Day 6 (or Day 4-7) in culture.

Expanded cell composition was determined by flowcytometry analysis for NK cells (CD3-CD56+), αβ T cells (CD3+TCRVαβ+), pan γδ T cells (CD3+TCRVγδ+) and Vδ1 (CD3+TCRVδ1+), Vδ2 (CD3+TCRVδ2+), and non-Vδ1&Vδ2 (CD3+Vδ1TCR-Vδ2TCR-αβTCR−) T cell subsets. Activation (CD69, NKG2D) and inhibitory receptor expression, effector and memory markers, were determined by multichannel flow cytometry analysis. Surface CAR expression was determined by CD34 QBEND/10 staining, and TGFβR2 anti-human TGFβRII staining. All antibodies were purchased from Biolegend except for TCRVγδ (Miltenyi) and CD34 (Abnova).

### Anti-CD2 Agonist Antibody Enhances the Expansion of Activated γδ T Cells

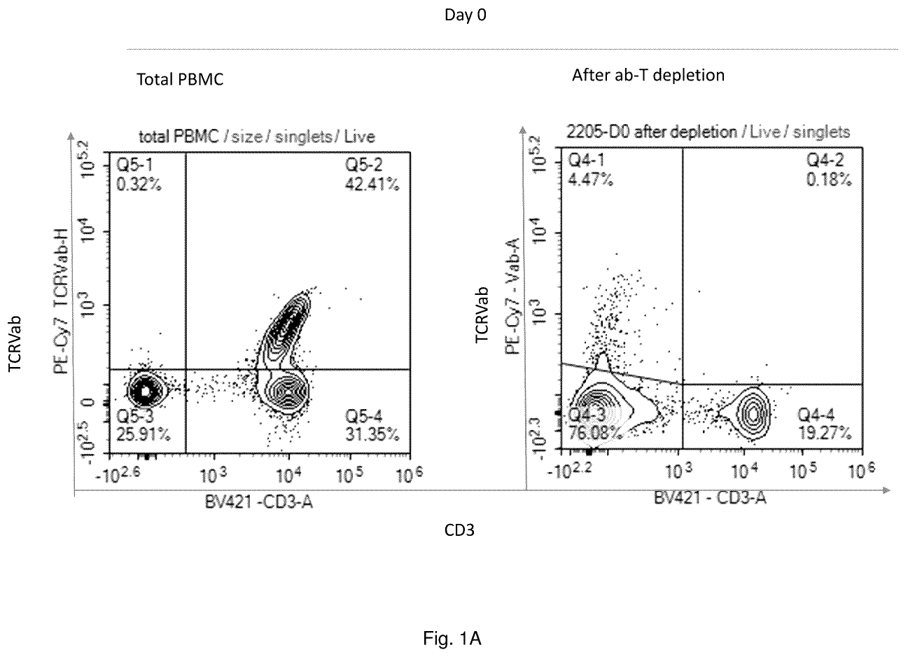

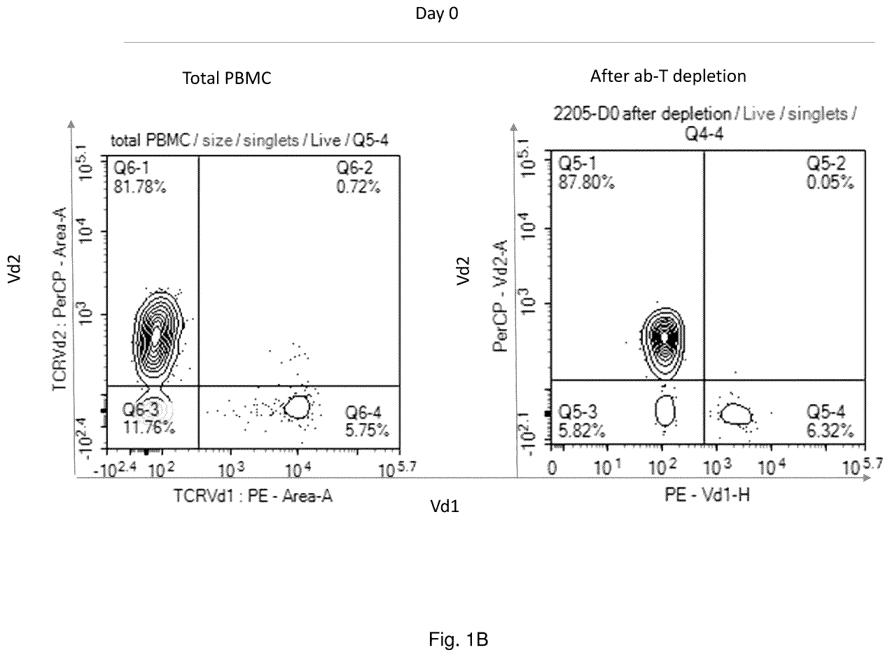

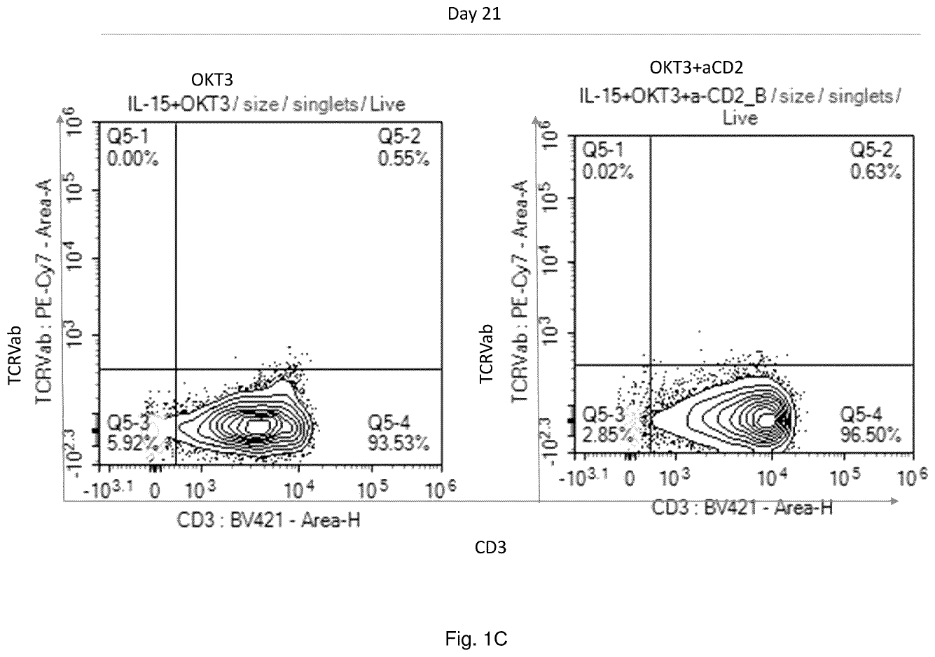

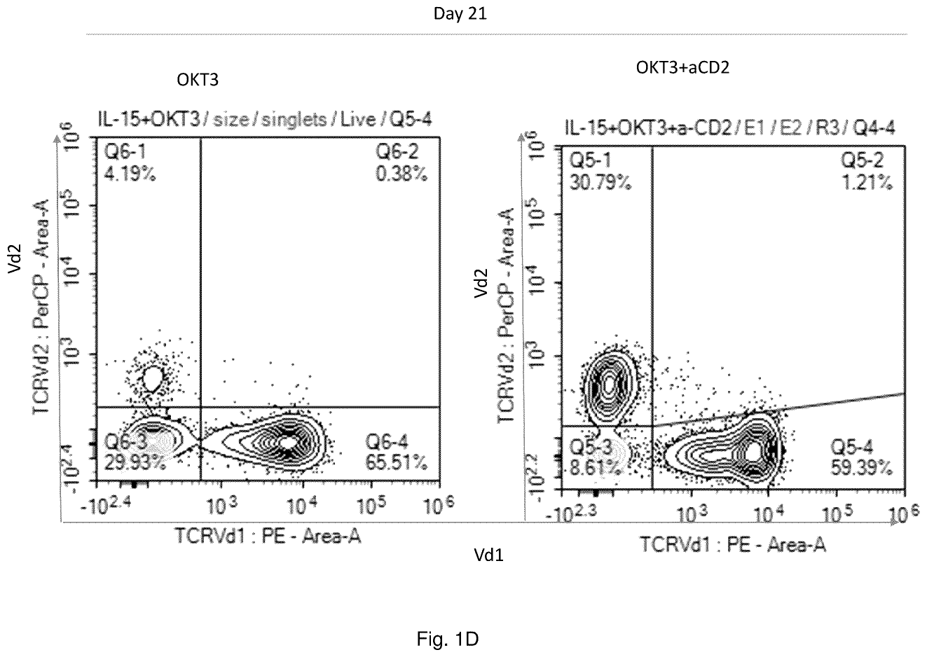

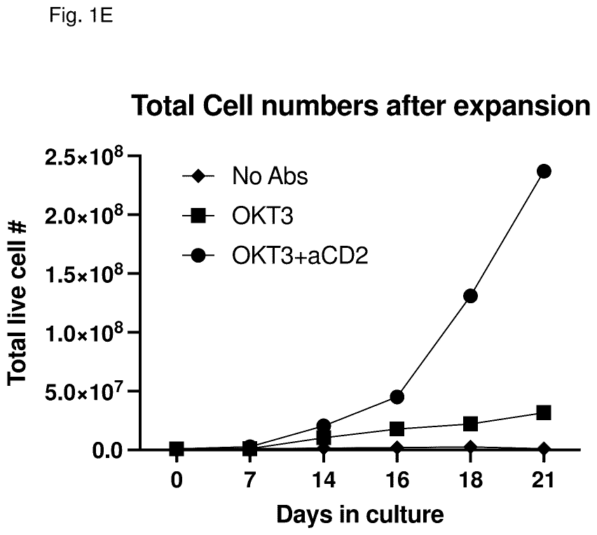

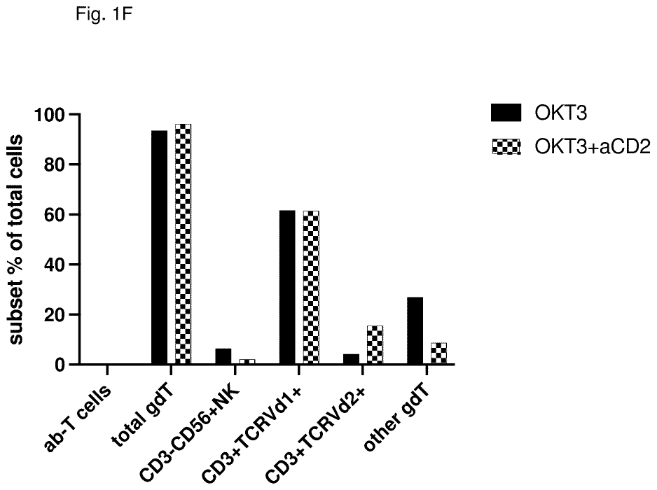

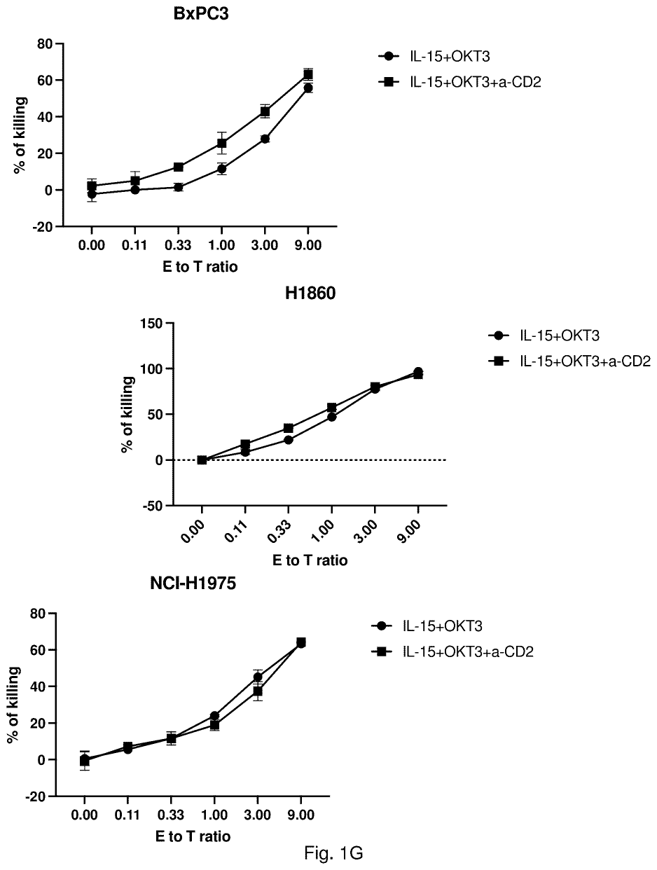

A frozen aliquot of 1×10⁶ αβ T-depleted leukapheresis donor sample was activated either with anti-CD3 Ab alone or with both anti-CD3 and anti-CD2 Abs. The addition of anti-CD2 agonist Ab resulted in significantly more expanded innate cells by Day 21 (FIG. 1E). Both conditions produced highly pure (~95%) γδ T cells, with minimal residue NK cells, and αβ T cells (FIGS. 1C, 1D, and 1F). The addition of CD2 agonist also impacted the composition of γδ T cells, which include the subtypes Vδ1, Vδ2, and non-Vδ1&Vδ2 T cells. The activation with CD2 agonists resulted in comparable levels of Vδ1, more Vδ2 and less Vδ1-Vδ2-T cells compared to that without CD2 activation (FIGS. 1C, 1D, and 1F). However, the innate cytotoxicity of γδ T cells expanded with or without anti-CD2 Ab was similar as determined in the co-culture with GFP-Luc-expressing pancreatic cancer cell line BxPC3, breast cancer cell line HCC1860, and lung cancer cell line NCI-H1975 at increasing E:T ratio of 1:9 to 9:1. Luciferase assay showed comparable tumor-specific lysis profile in 2 out of the 3 cell lines tested (FIG. 1G).

Expansion of Activated γδ T Cells with and IL-2 or IL-15 are Comparable

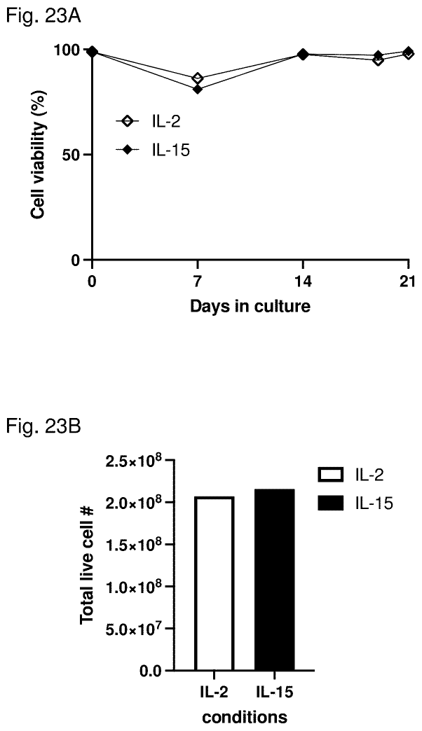

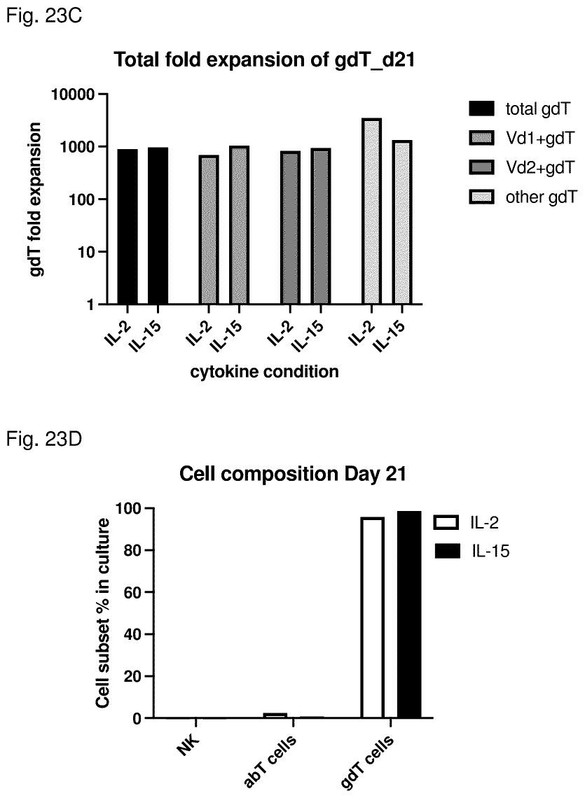

One million cryopreserved αβ T-depleted PBMCs were thawed, washed and activated in CTS culture complete medium containing 2.5% human AB serum, 1 μg/mL of OKT3 (Biolegend), 0.5 ug/mL of anti-CD2 Ab (Miltenyi) with 300 IU/ml of human IL-2 (R&D) or 7.5 ng/ml of human IL-15 (R&D). Medium or cytokine was supplemented every 3 or 4 days throughout the culture. Both cytokines expanded γδ T cells efficiently with similar cell viability (FIG. 23A), yield (FIG. 23B), fold of γδ T cell expansion (FIG. 23C) and purity (FIG. 23D) over 21-day culture.

TGFβR2 gRNA/Cas9 RNP Delivery Via Electroporation for Gene Knockout (KO) in Expanded γδ T cells.

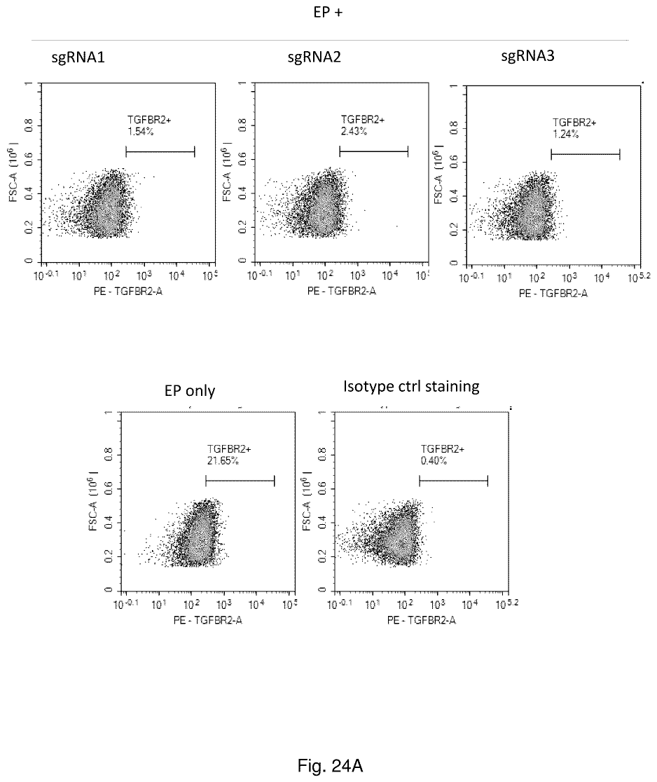

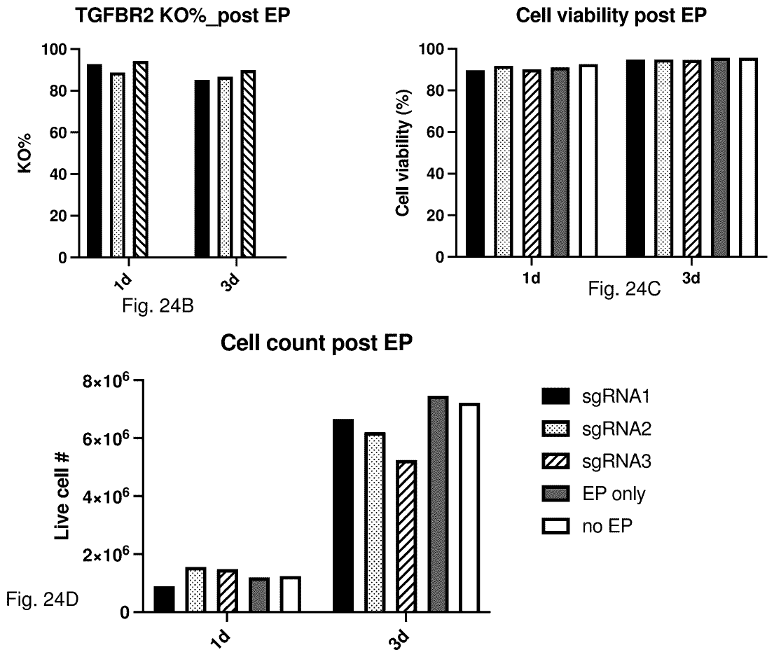

To disrupt TGFβR2 gene, 3 guide RNAs were identified targeting exon 4 in TGFβR2 gene. The synthetic gRNA (Genscript) was mixed with recombinant Cas9 protein (Genscript) at 2:1 molar ratio at room temperature to form ribonucleoprotein (RNP) complex. The RNP was then added to γδ T cells washed and resuspended in MaxCyte electroporation (EP) buffer to a final concentration of 3 uM. The mixture of γδ T cells (100-200 million cells/mL) and RNP was then transferred to an appropriate processing assembly for electroporation on MaxCyte ATX (MaxCyte). After Electroporation, cells were rested in incubator for 20 min and then returned to culture in prewarmed medium. One and 3 days later, surface expression of TGFβR2 was assessed by PE-anti-TGFβR2 Ab (Biolegend) in flow cytometry. TGFβR2 expression was reduced from approximately 2²% in γδ T cells electroporated without gRNA to 1-2% in γδ T cells with gRNAs (FIG. 24A), representing approximately 90% of gene KO efficiency (FIG. 24B), as calculated with the formula as: KO %= (TGFRBR2+% in EP buffer only—TGFRBR2+% in sgRNA)/TGFRBR2+% in EP buffer only *100%. In addition, TGFβR2 KO γδ T cells were highly viable (FIG. 24C) and proliferated actively starting from 1×10⁶ cells for EP on day 0 (FIG. 24D).

TABLE B
TGFβR2 guide RNA sequences

| # | TGFβR2 guide RNA sequence | SEQ ID NO: |
|---|---|---|
| 1 | TCTACTGCTACCGCGTTAAC | 142 |
| 2 | GCTTCTGCTGCCGGTTAACG | 128 |
| 3 | ACAGTGATCACACTCCATGT | 129 |

### Characterization of Expanded and Modified γδ T Cells

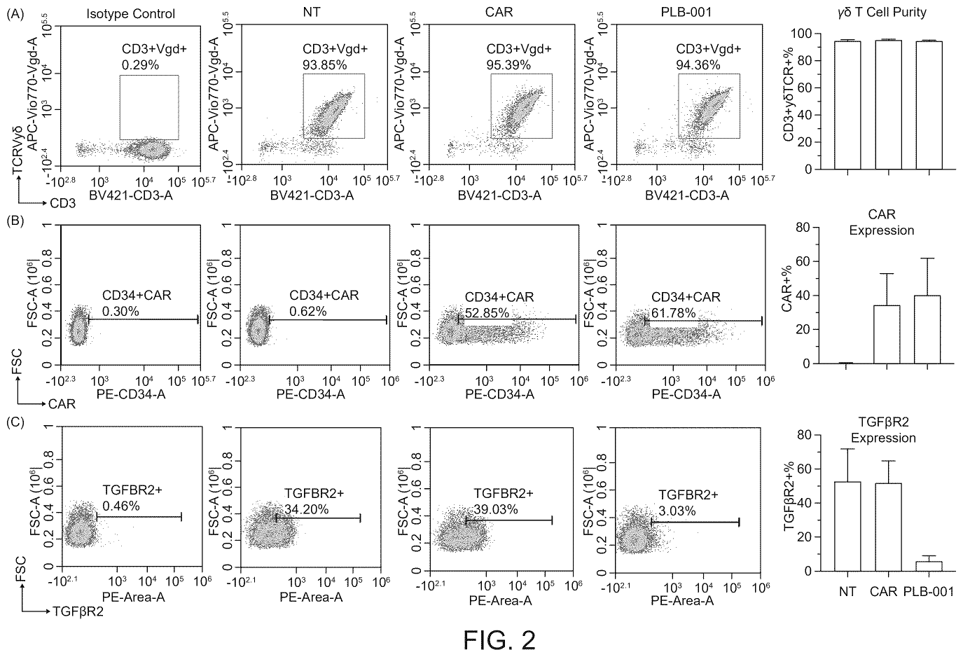

The transduction efficiency of TROP2-PD-L1 CAR-IL-15 retroviral vector varied in the range of 30%-70%. CRISPR/Cas9-mediated TGFβR2 gene editing was highly efficient, resulting in >90% knockout of TGFβR2 surface expression as determined by flow cytometry. In TGFβR2 KO, TROP2 PD-L1 CAR-IL-15 γδ T cells (PLB-001) derived from a single donor, 61% of the cells were stably transduced to express TROP2-PD-L1 CAR, and only 3% of cells have detectable surface TGFβR2 following TGFβR2-gRNA/Cas9 RNP-mediated gene editing. In comparison, TGFβR2+ cells were detected in 34%-39% of the cells without gene editing (FIG. 2).

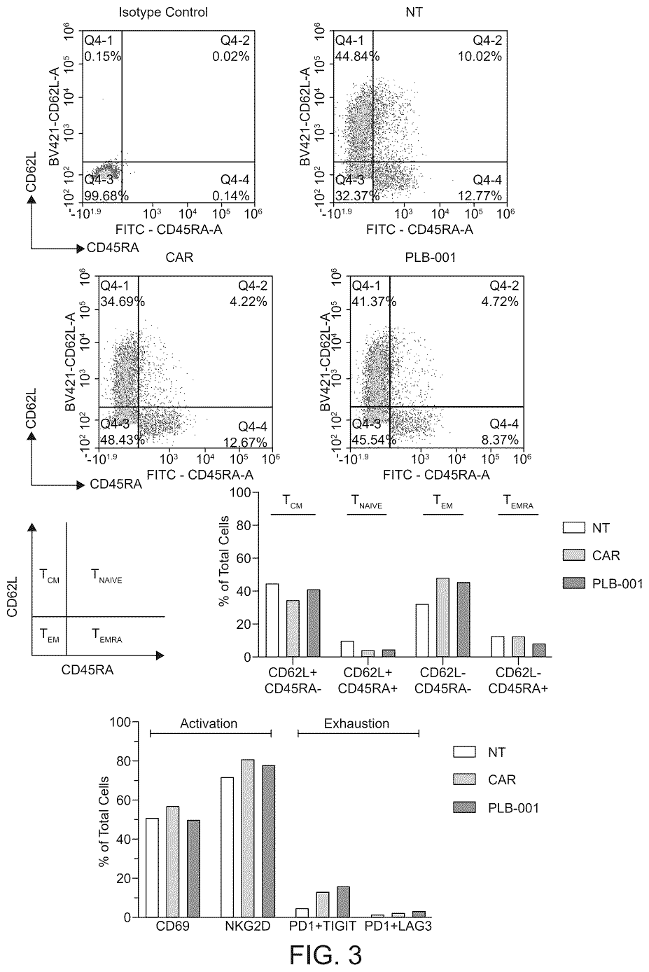

PLB-001 was highly pure with 94% γδ T cells, 41.4% displayed central memory (T_{CM}) phenotype (CD62L+CD45RA−) and 45.5% showed effector memory (Tem) phenotype (CD62L-CD45RA-). Compared to unmodified γδ T cells, retroviral vector transduction resulted in slightly reduced Tnaive population from 10% to 4.2%, with no additional impact from CRISRP/Cas9 gene editing. Both Tom and Tem have the potential for adaptive γδ T cell clonal expansion upon receptor occupation, along with the ability to home to peripheral tissues and exert effector function. In addition, the expanded γδ T cells were highly active, with a majority of the cells expressing the activation markers CD69 (50%) and NKG2D (80%), while a minority of the cells express exhaustion markers PD-1&TIGIT (17%), which is higher than that in unmodified γδ T cells (4.8%) (FIG. 3). TROP2-PD-L1 CAR-mediated cytotoxicity

As TROP2 is expressed on a wide range of tumors, tumor cell lines originating from TNBC (HCC70 and HCC1806), Breast adenocarcinoma (SKBR3), Lung adenocarcinoma (HCC827 and NCI-1975), Pancreatic cancer (BXPC3 and HPAF-II), Ovarian cancer (SKOV3) and Gastric cancer (NCI-N87) were purchased from ATCC (Manassas, VA), as well as K562 derived from chronic myeloid leukemia that does not express either TROP2 or PD-L1. Tumor cell lines were then modified to stably express eGFPFfluc by transduction with lentiviral vectors (BPS Bioscience, San Diego, CA). Surface expression levels of TROP2 and PD-L1 were confirmed and quantified by flow cytometry analysis with anti-human TROP2 antibody and anti-human PD-L1 antibody (Biolegend, San Diego, CA) and BD QUANTIBRITE beads (BD Biosciences, San Jose, CA).

The cytotoxicity of PLB-001, TROP2-PD-L1 CAR-IL-15 γδ T cells (CAR γδ T) and unmodified γδ T cells (NT) against co-cultured GFP-Luc-labeled tumor cell lines were accessed by luciferase activity assay. Luciferase activity was quantified using firefly luciferase HTS system (Sigma, St Louis, MO). Tumor luciferase activity percentage was calculated relative to tumor target only. Tumor-specific lysis was calculated using the formula: 100×[tumor only-sample)/tumor only. Tumor cell killing also was monitored via BIOTEK CYTATION 5 cell imaging multimode reader (Agilent, Santa Clara, CA).

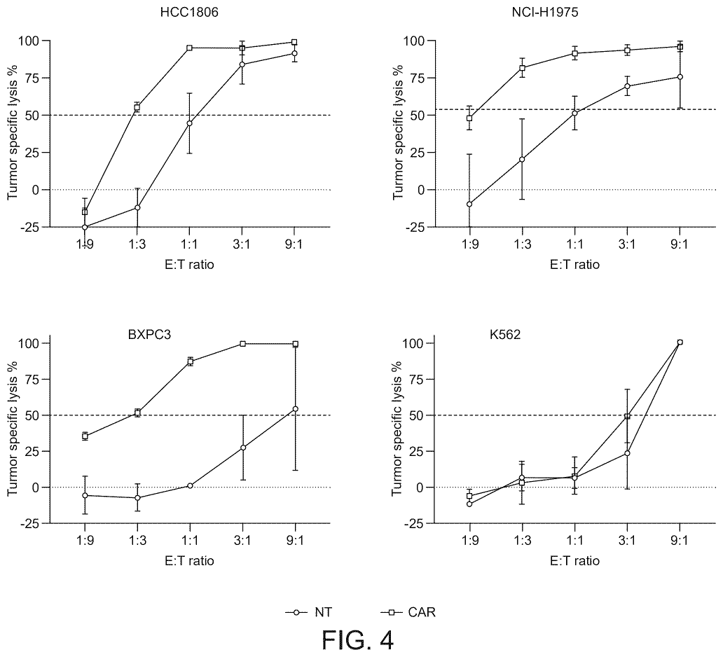

Following a 7 day coculture of γδ T cells with HCC1806, NCI-H1975, BXPC3 or K562 at increasing Effector: Target (E:T) ratios from 1:9 to 9:1, CAR γδ T cells showed potent cytotoxicity against TROP2+PD-L1+tumor cells, achieving approximately 50% of tumor lysis (ED50) at an E:T ratio of 1:9 (in NCI-H1975) or 1:3 (in HCC1806 and BXPC3). In the absence of IL-15 supplement, NT γδ T cells also demonstrate innate toxicity towards tumor cells, albeit less potent than TROP2 PD-L1 CAR-IL-15 γδ T cells, requiring higher E:T ratio of 1:1 (in HCC1806 and NCI-H1975) or 9:1 (in BXPC3) to reach 50% of tumor lysis. In coculture with TROP2-PD-L1-CML cell line K562, the CAR target-independent innate killing activity was comparable between CAR γδ T cells and NT γδ T cells, both reached 50% of tumor lysis at an E:T ratio at 3:1-9:1 (FIG. 4).

### TGFβR2 KO Significantly Improves the Anti-Tumor Activity of PLB-001

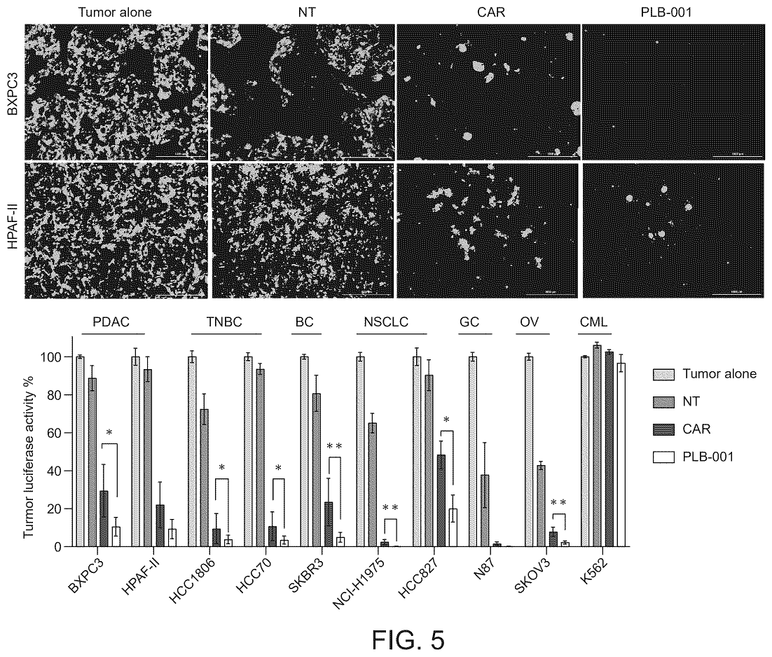

To determine the anti-tumor activity PLB-001 on broad tumor types, a wide variety of GFP-Luc-tumor cell lines derived from pancreatic cancer (BXPC3 and HPAF-II), TNBC (HCC70 and HCC1806), breast adenocarcinoma (SKBR3), lung adenocarcinoma (HCC827 and NCI-1975), gastric cancer (NCI-N87), and ovarian cancer (SKOV3) were tested, all of which express varying degrees of surface TROP2 and PD-L1. The tumor cells were cocultured with γδ T cells expanded from 2 healthy donors at an E:T ratio of 1:2 up to day 5. As shown in a representative fluorescent imaging of pancreatic cell lines BXPC3 and HPAF-II on Day 4, GFP-expressing tumor cells were killed extensively by CAR γδ T cells and were nearly eliminated by PLB-001. The tumor cell lysis was further quantified by luciferase assay on Day 5, which consistently showed robust tumor lysis by CAR γδ T cells, which were further significantly enhanced by PLB-001 (FIG. 5).

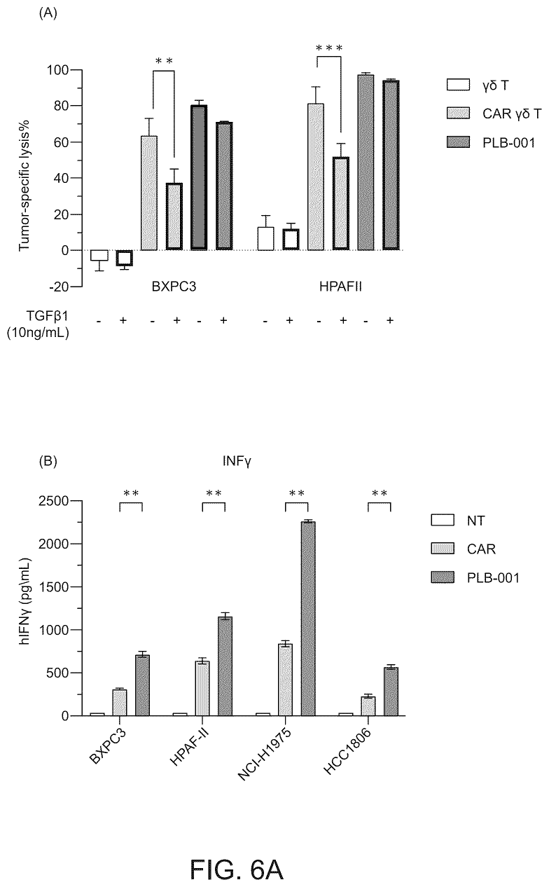

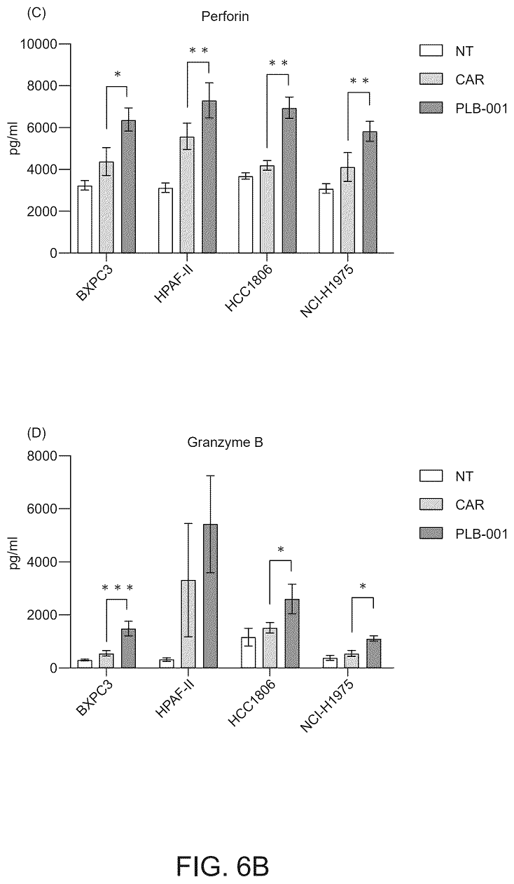

To determine the resistance of PLB-001 against TGFβ1-mediated suppression, 10 ng/ml of TGFβ1 was added to the serum-free coculture of γδ T cells and GFP-Luc-BXPC3 or HPAF-II pancreatic tumor cells at E:T ratio of 1:1. The tumor-specific lysis as determined by luciferase assay on Day 2 showed that PLB-001 maintained comparable tumor killing activity irrespective of the presence of TGFβ1, whereas CAR γδ T cells showed significantly reduced tumor lysis in the presence of 10 ng/ML of TGFβ1 (FIG. 6A, Panel A). The enhanced tumor killing activity of PLB-001 was consistent with the finding of significantly higher levels of pro-inflammatory cytokine INFγ, cytolytic enzymes perforin and granzyme B in the supernatant of the coculture, as determined by ELISA (R&D), of tumor cells with PLB-001 than with CAR γδ T cells (FIG. 6A, Panel B, and FIG. 6B Panels C and D). The results indicate that the activity of CAR γδ T cells are negatively impacted significantly by TGFβ present in the culture medium of the tumor cell lines, whereas TGFβR2 KO preserved PLB-001 activity resistant to the suppression of TGFβ, characteristic of solid tumor TME.

### Anti-Tumor Activity of PLB-001 in 3D Pancreatic Tumor Spheroid Model

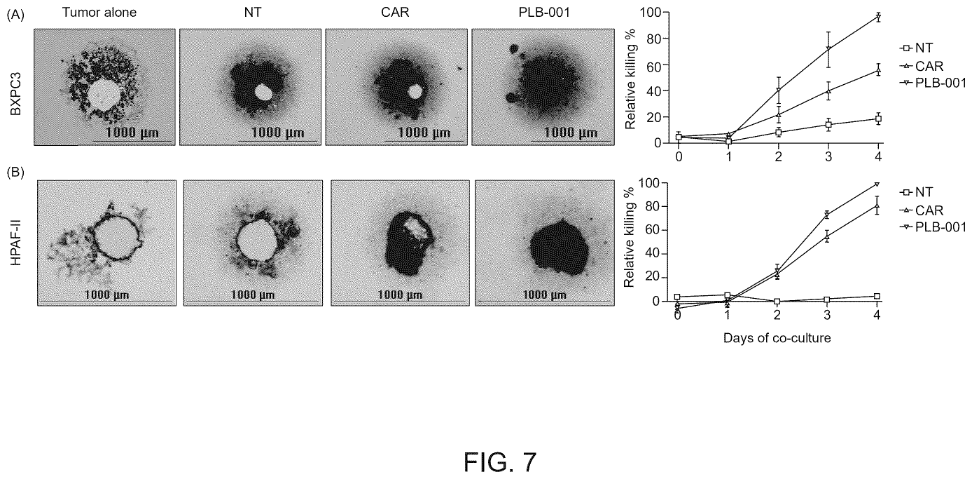

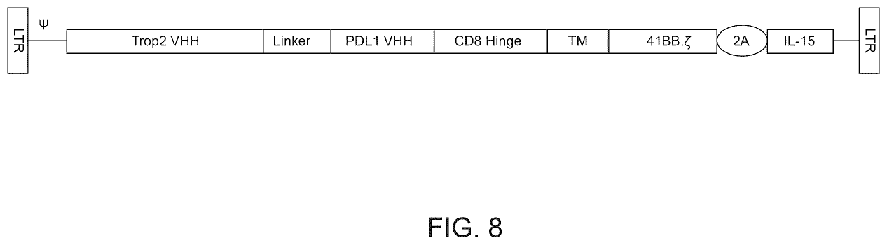

Tumor spheroids were seeded with 5,000 cells of GFP-Luc-expressing pancreatic tumor cell line BxPC3 and HPAF-II per well on ultra-low attachment PRIMESURFACE 3D culture round bottom 96 wells plate (S-bio). When the tumor spheroids were established after 3 days, 5,000 or 15,000 of NT γδ T cells, TROP2 PD-L1 CAR γδ T cells or PLB-001 were added for coculture. Tumor spheroid killing was monitored daily, and total green fluorescence intensity was quantified on BIOTEK CYTATION 5 cell imaging multimode reader (Agilent, Santa Clara, CA). NT γδ T cells showed limited tumor killing activity, which was dramatically increased in CAR γδ T cells, and which was further significantly enhanced in PLB-001. PLB-001 effectively infiltrated tumor spheroids and eliminated all detectable tumor cells over 4 days (FIG. 7).

### Serial Tumor Killing Activity of PLB-001 Following Repeat Tumor Challenges

On Day-1, multiple plates of 5,000 cells of GFP-Luc-labeled tumor cell lines NCI-H1975, HPAFII or BXPC3 per well were seeded. On Day 0, the tumor cells were either left untreated (TC-only) or co-cultured with 20,000 cells of PLB-001 or unmodified γδ T cells (NT). On Days 3, 7 and 11, Duplicate plates were used to image tumor green fluorescence intensity on BIOTEK CYTATION 5, quantify tumor-specific killing by luciferase assay, T cell counts and phenotyping by flow cytometry. In addition, on Days 3 and 7, 5,000 fresh tumor cells per well were added to the unused plates to initiate a new round of tumor challenge.

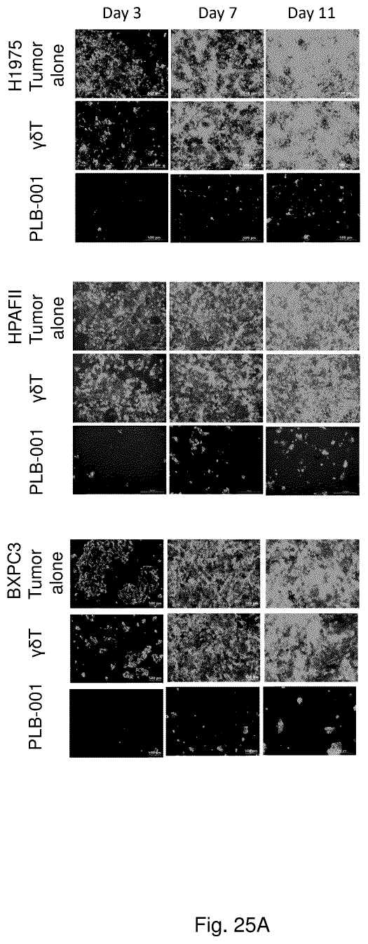

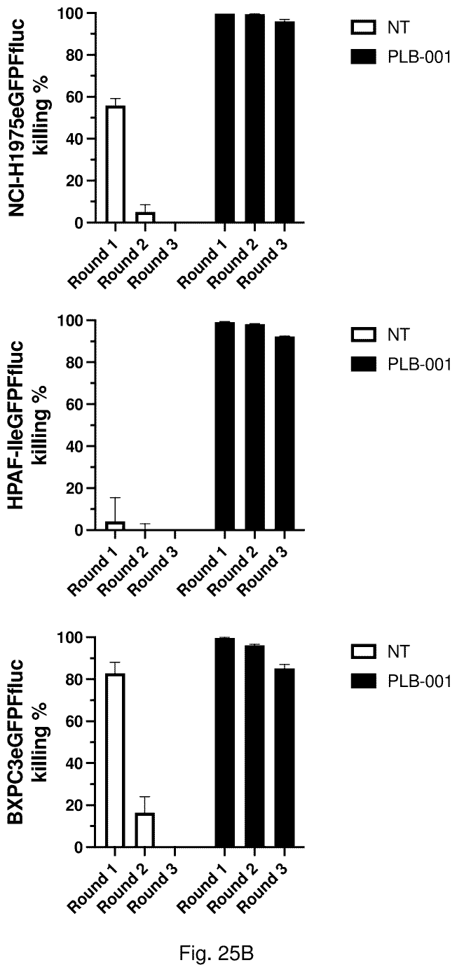

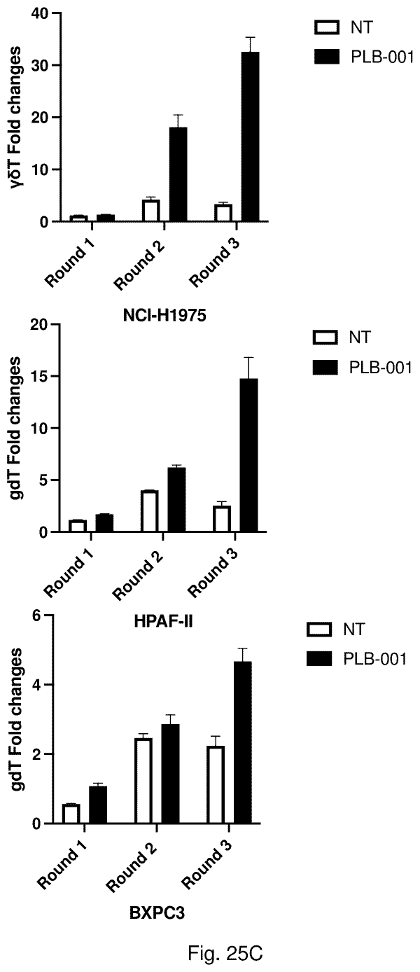

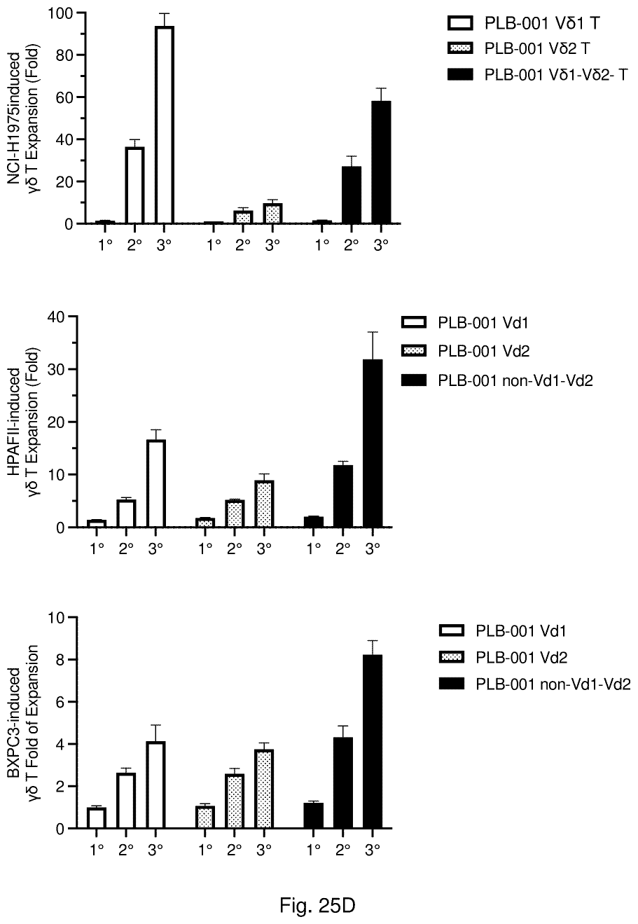

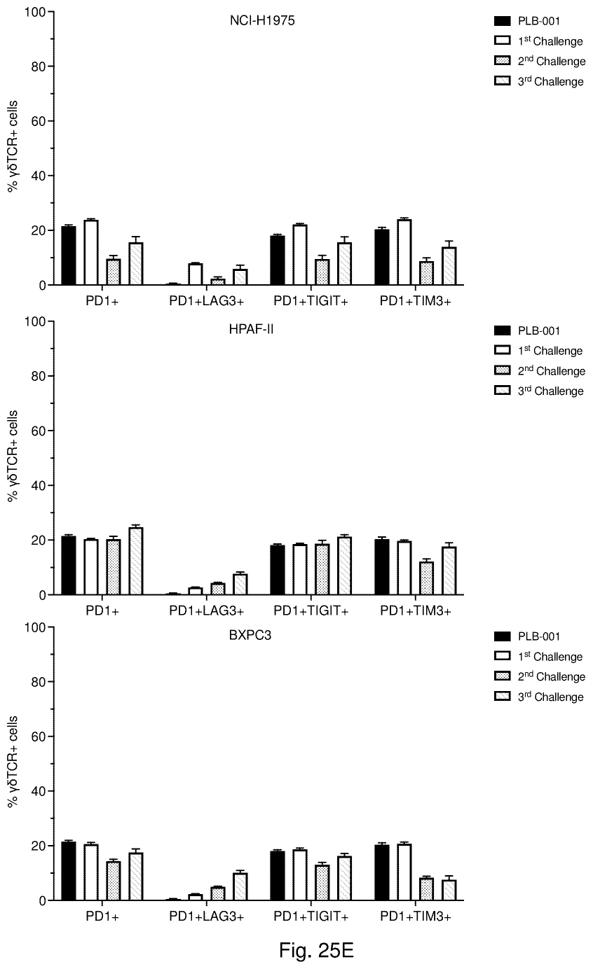

PLB-001 demonstrated robust serial killing capability, efficiently eliminating GFP-Luc-labeled tumor cell lines NCI-H1975, HPAFII or BXPC3 compared with unmodified γδ T cells (FIG. 25A), retaining >85% tumor lysis throughout 3 tumor challenges (FIG. 25B). The durable anti-tumor activity of PLB-001 is supported by the γδ T cell expansion following tumor antigen engagement, reaching 4-30-fold in response to the different tumor cells by Day 11 (FIG. 25C). Notably, subsets Vδ1 and Vδ1-Vδ2-T cells showed higher expansion capacity than Vδ2 (FIG. 25D). Importantly, following 3 rounds of tumor challenges, PLB-001 retains activity and expresses only low levels of exhaustion markers including PD1+LAG3+ (FIG. 25E).

### Tumor-Homing, Infiltration and Anti-Tumor Activity of PLB-001 in Tumor Xenograft Model in Vivo

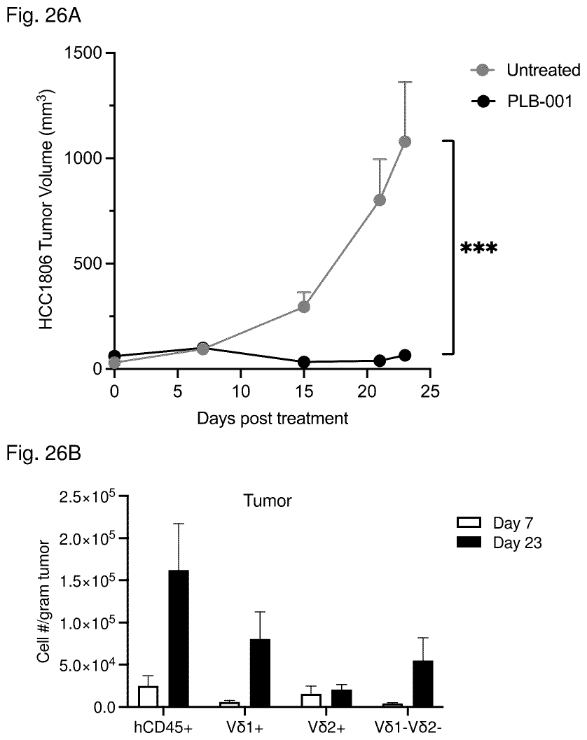

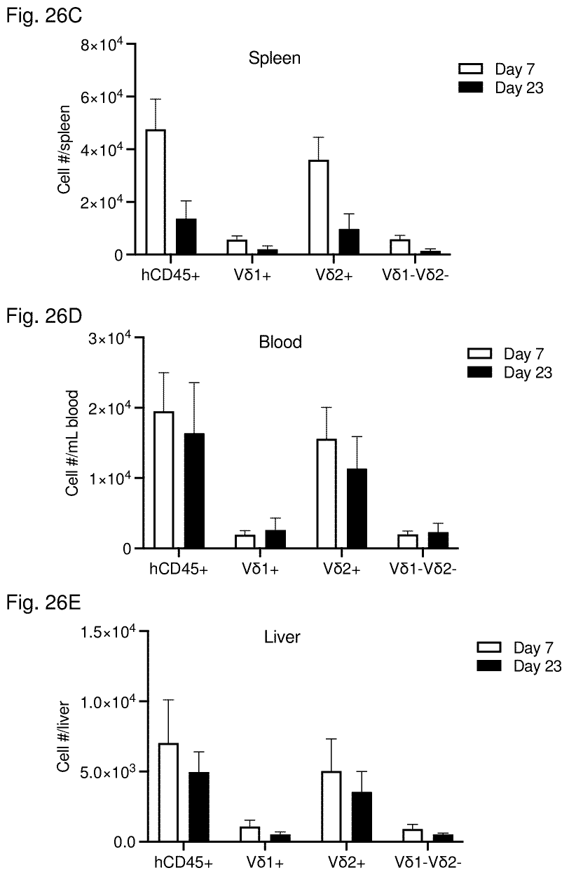

PLB-001 also demonstrated capability for homing to, infiltrating and proliferating in tumors in vivo, leading to significant control of tumor growth in subcutaneous TNBC xenograft model in 6 to 8-week-old NOD/SCID/IL-2γR−/−(NSG) mice (Jackson laboratory). 2×10⁶ of GFP-Luc-HCC1806 cells suspended in PBS were mixed with matrigel at 1:1 in a total of 100 μL, and subcutaneously injected to a flank of the mouse and two flanks were injected per mouse. After the tumor was established to the size of 50-100 mm³, 6 mice were treated with 10×10⁶ cells of PLB-001 in 100 μL of PBS via intravenous injection and 3 mice were left untreated. Mouse body weight and tumor size were monitored weekly. Three PLB-001-treated mice were sacrificed on Day 7 and the remaining animals were sacrificed on Day 2³ to assess the tissue distribution and proliferation of PLB-001. As shown in FIG. 26A, tumors did not grow in PLB-001-treated mice in contrast to the significant tumor growth in untreated animals. PLB-001 are detected in tumors, spleen, blood and liver (FIG. 26B-E) on both Days 7 and 2³. However, robust expansion of PLB-001 observed only in tumors, and mainly in the subsets of Vδ1+ and Vδ1-Vδ2-T cells (FIG. 26B), consistent with the finding in vitro upon repeat tumor cell challenge. Given the tissue-resident nature of Vδ2-T cells, the result suggests the potential of PLB-001 to retain in tissue for long-term tumor surveillance. Furthermore, the lack of PLB-001 expansion in normal tissues also highlights the safety feature of γδ T cells in the setting of allogeneic cell therapy. γδ T cells do not recognize MHC I, thus are less likely to develop potential graft-vs-host diseases. Indeed, mice maintained normal body weight and showed no clinical sign of GvHD in this study.

In summary, a process to generate healthy donor-derived, TGFβR2 gene edited, TROP2-PD-L1 CAR IL-15-expressing multi-subsets of γδ T cells (PLB-001) was developed, which has shown robust anti-tumor activity against TROP2+PD-L1+tumor cell line culture and 3D tumor spheroids derived from wide variety of tumor types, resistant to the suppression of TGFβ present in the culture medium. PLB-001 showed serial tumor-killing activity and robust expansion upon tumor cell challenge in vitro, resulting in significant reduction of tumor burden in tumor xenograft model in vivo.

### Example 2: Generation of Anti-PD-L1 VHH (Immunized Alpaca Phage Library Construction, Panning And Screening)

An adult healthy alpaca was immunized with recombinant human PD-L1-FC protein (GENBANK accession no. Q9NZQ7):

(SEQ ID NO 130)
FTVTVPKDLYVVEYGSNMTIECKFPVEKQLDLAALIVYWEMEDKNIIQFVHGEEDLKVQHSSYRQR
ARLLKDQLSLGNAALQITDVKLQDAGVYRCMISYGGADYKRITVKVNAPYNKINQRILVVDPVTSEH
ELTCQAEGYPKAEVIWTSSDHQVLSGKTTTTNSKREEKLFNVTSTLRINTTTNEIFYCTFRRLDPEE
NHTAELVIPELPLAHPPNERTHLVILGAILLCLGVALTFIFRLRKGRMMDVKKCGIQDTNSKKQSDTH
LEET

The animal boosted four times every two weeks. After confirming the serum titer against PD-L1, PBMC was isolated and used for phage display library construction. Anti-PD-L1 binders were isolated with the immunized phage display library using liquid phase Biopanning on biotinylated human PD-L1. Stringency was increased during successive rounds by decreasing antigen concentration, increasing the number and duration of washes, and/or changing the duration of selections. The panning process was repeated three to four times until the output phage enrichment was identified. The eluted phages were diluted and spread on a petri dish to grow single colonies. Individual phage clones were selected and amplified. 384 single colonies were selected and tested for specificity by ELISA. 264 clones were positive compared to the negative control. After sequencing the positive clones, three unique sequences were obtained based on amino acid sequences.

TABLE 1
Anti-PD-L1 VHH clone sequences

| SEQ ID | | |
|---|---|---|
| NO | Notes | Sequences |
| 1 | anti-PD-L1 #PD1 | DVQLQESGGGLVQAGGSLRLSCAASESIASINVMGWYRQAPGKQRELV |
| | VHH amino acid | AIITNRGTTSYPDSVKGRFTISRDNGKNTVYLQMNTLKPEDTAVYYCNAP |
| | sequence | WTDGINYWGKGTQVTVSS |
| 2 | anti-PD-L1 #PD1, | ESIASINV |
| | HVR-H1 | |
| | polypeptide | |
| 3 | anti-PD-L1 #PD1, | ITNRGTT |
| | HVR-H2 | |
| | polypeptide | |
| 4 | anti-PD-L1 #PD1, | NAPWTDGINY |
| | HVR-H3 | |
| | polypeptide | |
| 5 | anti-PD-L1 #PD2 | DVQLQESGGGLVEPGGSLRLSCAASGFTFNHYGMSWVRQAPGKGLQW |
| | VHH amino acid | VSTLFTTGSSQYANSVKGRFTISRDAAENTLYLHMNNLKPEDTAVYFCA |
| | sequence | KGVGLGSERGPGTQVTVSS |
| 6 | anti-PD-L1 #PD2, | GFTFNHYG |
| | HVR-H1 | |
| | polypeptide | |
| 7 | anti-PD-L1 #PD2, | LFTTGSS |
| | HVR-H2 | |
| | polypeptide | |
| 8 | anti-PD-L1 #PD2, | AKGVGLGSE |
| | HVR-H3 | |
| | polypeptide | |
| 9 | anti-PD-L1 | DVQLQESGGGLVQAGDSLGLSCAASGRAFSLFEMAWYRQAPGKAREF |
| | #PD3 VHH amino | VAAIMWRGGHTVYADSVKGRSTISSDNTKNTVYLQMNSLKPEDTAIYYC |
| | acid sequence | RGKVVGSDYWGQGTQVTVSS |
| 10 | anti-PD-L1 #PD3, | GRAFSLFE |
| | HVR-H1 | |
| | polypeptide | |
| 11 | anti-PD-L1 #PD3, | IMWRGGHT |
| | HVR-H2 | |
| | polypeptide | |
| 12 | anti-PD-L1 #PD3, | RGKVVGSDY |
| | HVR-H3 | |
| | polypeptide | |

### Example 3: Application of PD-L1 Binders to Cell Therapy

Anti-PD-L1 VHH, when expressed on the immune cell surface as a binder in CAR fusion protein, can bind PD-L1-positive tumor cells resulting in activation of immune cells to kill tumor cells. CAR vectors, comprising anti-PD-L1 VHH (ID #PD1, PD2 and PD3), CD8α hinge and transmembrane domain, 4-1BB and CD34 signaling domains, and human IL-15 separated by a T2A sequence, were constructed by cloning DNA fragments containing all domains into a gamma-retroviral plasmid (Biovec pharma, Quebec, Canada).

The CD8 hinge amino acid sequence included in this construct is:

(SEQ ID NO: 137)
TTTPAPRPPTPAPTIASQPLSLRPEACRPAAGGAVHTRGLDFACD

The CD8 transmembrane domain amino acid sequence included in this construct is:

(SEQ ID NO: 138)
IYIWAPLAGTCGVLLLSLVITLYC

The 4-1BB signaling domain amino acid sequence included in this construct is:

(SEQ ID NO: 139)
KRGRKKLLYIFKQPFMRPVQTTQEEDGCSCRFPEEEEGGCEL

The CD3ζ signaling domain amino acid sequence included in this construct is:

(SEQ ID NO: 140)
RVKFSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR

The T2A amino acid sequence included in this construct is:

(SEQ ID NO: 141)
EGRGSLLTCGDVEENPGP

Human IL-15 amino acid and nucleic acid sequences are provided below:

(SEQ ID NO: 131)
MRISKPHLRSISIQCYLCLLLNSHFLTEAGIHVFILGCFSAGLPKTEANWVNVISDLKKIEDLIQSMHID
ATLYTESDVHPSCKVTAMKCFLLELQVISLESGDASIHDTVENLIILANNSLSSNGNVTESGCKECEE
LEEKNIKEFLQSFVHIVQMFINTS
(SEQ ID NO: 132)
atgagaatttcCaaaccacatttgaggagcatttccatccagtgctacttgtgtttacttctaaacagtcattttctaactgaagctggcat
tcatgtcttcattttgggctgtttcagtgcagggcttcctaaaacagaagccaactgggtgaatgtaataagtgatttgaaaaaaattgaag
aCcttattcaatctatgcaCattgatgctactttatatacggaaagtgatgttcaccccagttgcaaagtaacagccatgaagtgctttctc
ttggagttacaagttatttcacttgagAGCggagatgcaagtattcatgatacagtagaaaatctgatcatcctagcaaacaacagtttgtc
ttctaatgggaatgtaacagaatctggatgcaaagaatgtgaggaactggaggagaagaacatcaaggaatttttgcagagttttgtGcata
ttgtccaaatgttcatcaacacttct

Retroviral vectors were generated via transient transfection of CAR vector into the 293vecRDF114 packaging cell line (Biovec pharma, Quebec, Canada) using gene juice (Sigma, St Louis, MO). Retroviral vector supernatant was harvested at 48 and 72 hrs post-transfection and stored at −80° C. until use.

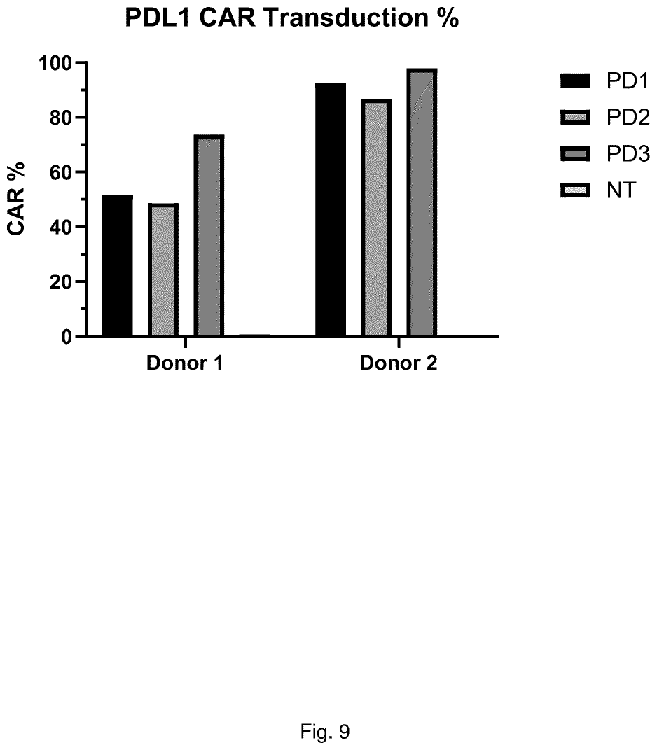

Retroviral vectors encoding different PD-L1 binder-derived CARs were then used to generate PD-L1 CAR γδ T cells. The activated γδ T cells from two healthy donors were efficiently transduced with CAR retroviral vectors as assessed by flow cytometry (FIG. 9).

### Example 4: Evaluation of Anti-Tumor Activity of PD-L1 CAR-IL-15 γδ T Cells

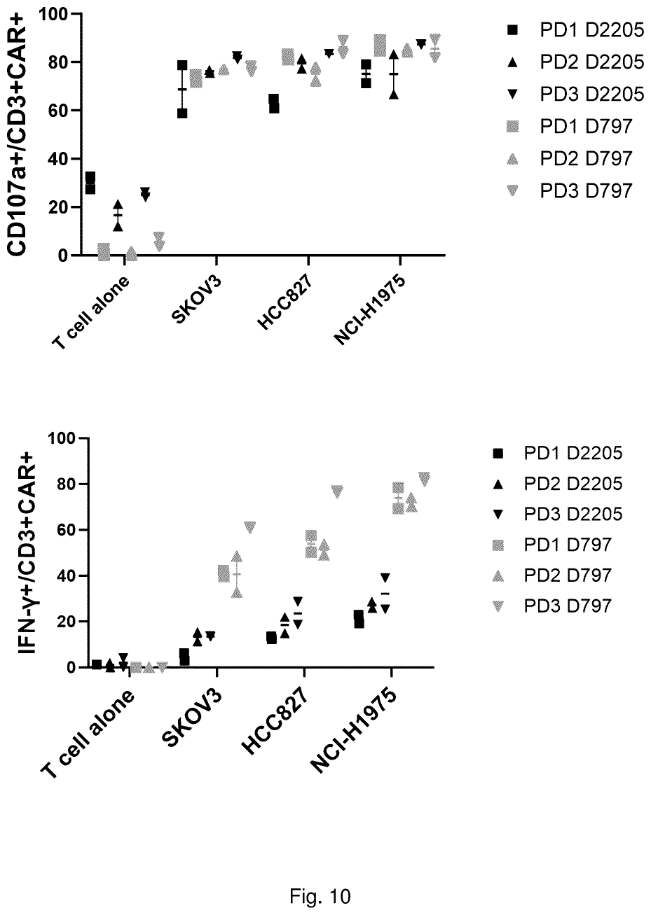

Degranulation and cytokine secretion of PD-L1 CAR γδ T cells co-cultured with tumor cells γδ T cells modified with different PD-L1 binder CARs were co-cultured overnight with or without a panel of PD-L1-positive tumor cell lines (SKOV3, HCC827, and NCI-H1975). Anti CD107a-PE antibody (Biolegend, San Diego, CA) was added during the first hour followed by the secretion inhibitor monensin (2 μM, Biolegend) and Brefeldin A (5 μg/ml, Biolegend) treatment. Cells were then washed and stained with anti-CD3 BV421 (Biolegend), and anti-CAR CD34 QBEND/10. Flow cytometer (Sony SA3800) was used to detect the CD107a as an indicator for degranulation in CD3+CAR+γδ T cells (FIG. 10, top panel). Production of IFN-γ by CD3+CAR+γδ T cells were analyzed by intracellular staining (FIG. 10, bottom panel). Antigen engagement with PD-L1-positive tumor cells induced prevalent degranulation and upregulation of IFN-γ expression, indicating that PD-L1 binders are fully functional in activating CAR γδ T cells in response to tumors.

### Antitumor Cytotoxic Effects of PD-L1 CAR γδ T Cells

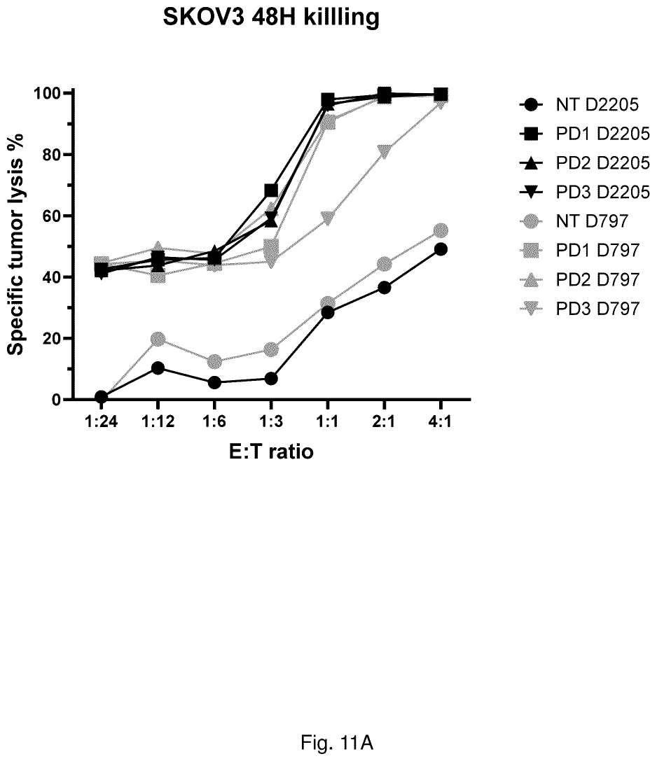

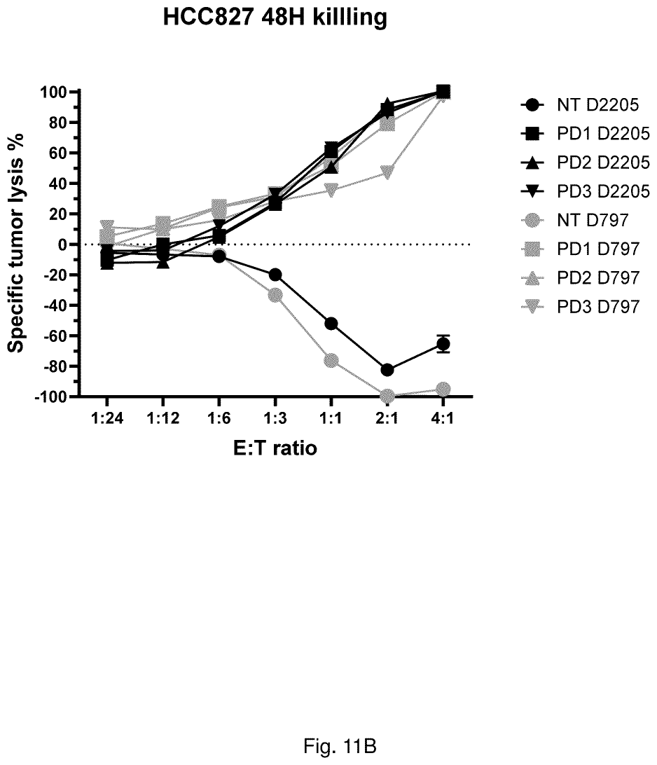

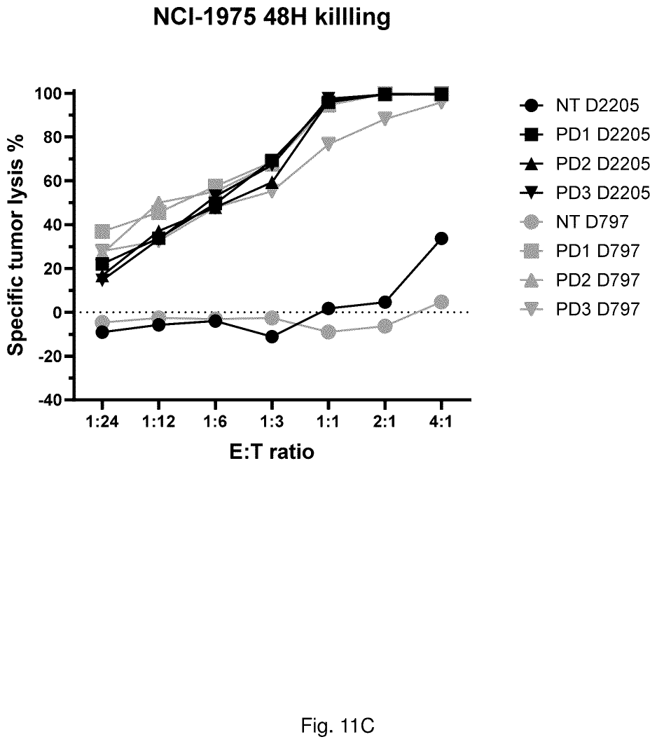

The antitumor cytotoxic activities of PD-L1 CAR γδ T cells compared with unmodified γδ T cells (NT) derived from two donors were assessed against a panel of PD-L1-positive cancer cell lines (i.e., SKOV3, HCC827, and NCI-H1975). Following 48 hr co-culture with GFP-Luc-expressing respective tumor cell lines at the indicated E:T ratio from 1:24 to 4:1, PD-L1 CAR γδ T cells, in comparison to NT, demonstrated significantly enhanced killing of PD-L1-postive tumor cell lines SKOV3 (FIG. 11A), HCC827 (FIG. 11B), and NCI-H1975 (FIG. 11C). Unmodified γδ T cells also showed innate tumor killing capability towards ovarian cancer cell line SKOV3 at high E:T ratio (FIG. 11A).

### Anti-Tumor Activity of PD-L1 CAR γδ T Cells in 3D Tumor Spheroid Model

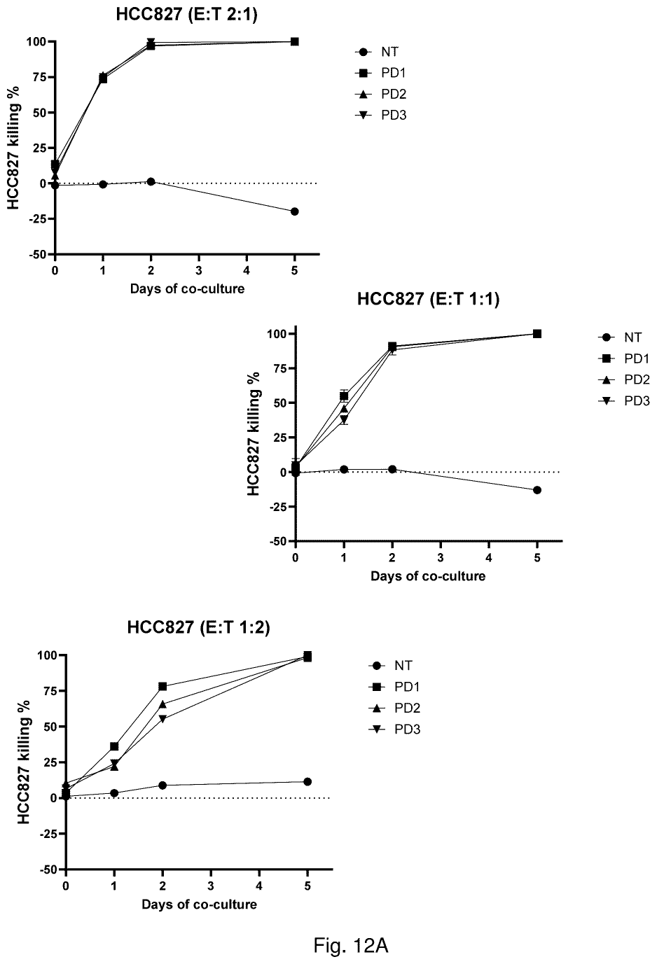

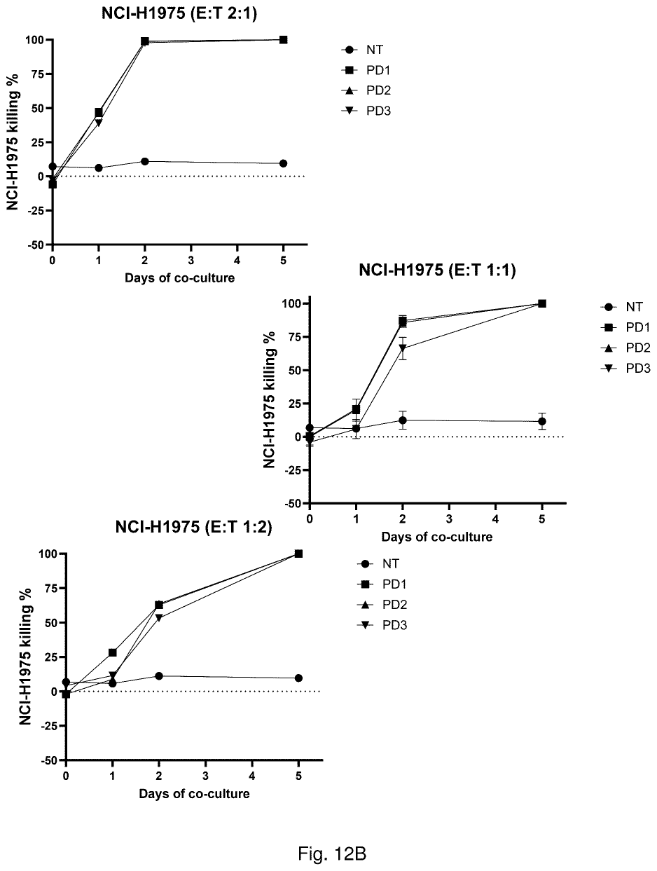

Tumor spheroids were seeded with 5,000 cells of GFP-Luc-expressing lung adenocarcinoma cell lines HCC827 and NCI-H1975. When the tumor spheroids were established after 3 days, 10,000, 5,000, or 2,500 PD-L1 CAR γδ T cells or NT γδ T cells were added for coculture. Tumor spheroids were monitored, and total green fluorescence intensity was quantified on BIOTEK CYTATION 5 cell imaging multimode reader (Agilent, Santa Clara, CA) daily up to 5 days. NT γδ T cells showed limited tumor killing activity, whereas PD-L1 CAR γδ T cells effectively infiltrated tumor spheroids and eliminated all detectable tumor cells over 5 days (FIGS. 12A, 12B).

### Example 5: Generation of Anti-TROP2 VHH (Immunized Alpaca Phage Library Construction, Panning, and Screening)

An adult healthy alpaca was immunized with recombinant human TROP2-FC protein (GENBANK accession no. P09758):

(SEQ ID NO: 133)
HTAAQDNCTCPTNKMTVCSPDGPGGRCQCRALGSGMAVDCSTLTSKCLLLKARMSAPKNARTLV
RPSEHALVDNDGLYDPDCDPEGRFKARQCNQTSVCWCVNSVGVRRTDKGDLSLRCDELVRTHHI
LIDLRHRPTAGAFNHSDLDAELRRLFRERYRLHPKFVAAVHYEQPTIQIELRQNTSQKAAGDVDIGD
AAYYFERDIKGESLFQGRGGLDLRVRGEPLQVERTLIYYLDEIPPKFSMKRLT

The animal was boosted three times every 21 days. Jugular blood was taken on day 7 after each boost and used to analyze the serum titer. After 3^{rd} and 4^{th} immunizations, the serum titer increased significantly which showed binding to human and cynomolgus TROP2 but not to control human protein even at 1:256,000 dilution by ELISA, and had higher binding of TROP2-CHOK1 cell compared to the TROP2-negative CHOK1 control cells.

After confirming successful immunization with TROP2 protein, PBMC was isolated and used for construction of a phage display library with the size of 2.42×10⁹ cfu. TROP2 VHH binders were isolated from the phage display library using solid phase panning on human TROP2-His protein and cell-based panning on the engineered, TROP2-expressing CHOK1 cells in two parallel discovery campaigns. Two to three rounds of panning and screening were conducted using 1011 alpaca VHH displayed phage. Stringency was increased during successive rounds by decreasing antigen concentration, increasing the number and duration of washes, and/or changing the duration of selections. The positively identified phages were diluted and spread on a petri dish to grow single colonies. Individual phage clones were selected and amplified. The panning on human TROP2-His protein resulted in 185 postive clones and 16 unique sequences. After three rounds of screening on TROP2 CHOK1 cells, a total of 384 clones were selected, of which 163 clones were TROP2-specific as tested by ELISA. After sequencing the positive clones, 9 unique sequences were obtained based on amino acid sequences. In combination, a total of 20 unique sequences were identified.

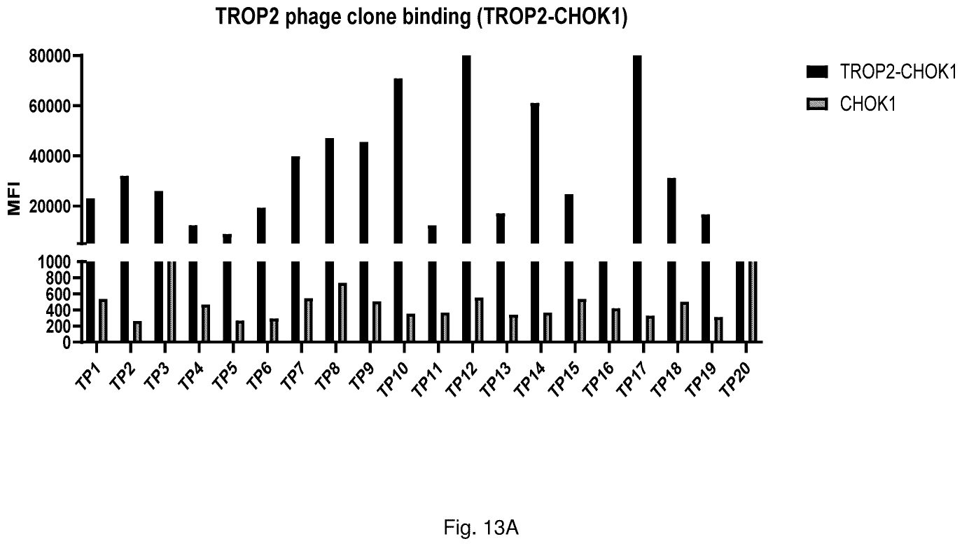

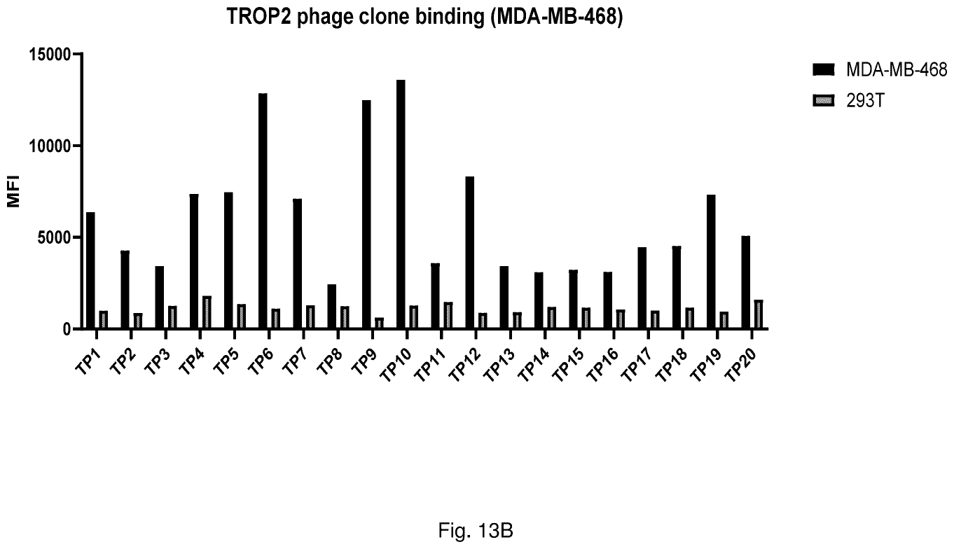

To confirm phage specificity and functionality, phages from 20 single colonies were used to stain TROP2-positive (TROP2-CHOK1 and MDA-MB-468) and TROP2-negative cells (CHOK1 and 293T) by flow cytometry. As shown in FIGS. 13A and 13B, there was increased binding of TROP2-CHOK1 and MDA-MB-468 cells, compared to the TROP2-negative CHO and 293T control cells, except for TP20 that showed comparable binding between TROP2-postive and -negative CHOK1 cells.

TABLE 2
Anti-TROP2 VHH clone sequences

| SEQ ID | | |
|---|---|---|
| NO | Notes | Sequences |
| 13 | anti-TROP2 | EVQVVESGGGLAQPGGSLRLSCAASGFWFSTYAMSWARQAPGKGLE |
| | #TP1 VHH amino | WVSGIYADGSATYYADSVKGRFTISRDNAKNTLYLHMNSLKPEDTALYY |
| | acid sequence | CAQGYSDYDRRVGGYDYWGQGTQVTVSS |
| 14 | anti-TROP2 TP1, | GFWFSTYA |
| | HVR-H1 | |
| | polypeptide | |
| 15 | anti-TROP2 TP1, | IYADGSAT |
| | HVR-H2 | |
| | polypeptide | |
| 16 | anti-TROP2 TP1, | AQGYSDYDRRVGGYDY |
| | HVR-H3 | |
| | polypeptide | |
| 17 | anti-TROP2 | EVQLVESGGGLAQPGGSLRLSCAASGFTFSTYAMSWARQAPGKGLEW |
| | #TP2 VHH amino | VSGIYADGSGTYYADSVKGRFTISRDNAKNTLYLQMNSLKPEDTALYYC |
| | acid sequence | AQGYSDYDRRLGEYDYWGQGTQVTVSS |
| 18 | anti-TROP2 TP2, | GFTFSTYA |
| | HVR-H1 | |
| | polypeptide | |
| 19 | anti-TROP2 TP2, | IYADGSGT |
| | HVR-H2 | |
| | polypeptide | |
| 20 | anti-TROP2 TP2, | AQGYSDYDRRLGEYDY |
| | HVR-H3 | |
| | polypeptide | |
| 21 | anti-TROP2 | QVQLVESGGGLAQPGGSLRLSCATSVFMSKTYMMSWYRQAPGKGLEW |
| | #TP3 VHH amino | VADISADGKLTYYDDAEEGFFLFSEDDHKNTVLLMMNLLKHDSTILRCCA |
| | acid sequence | WGDKDYDRRDWGHDNWVTVSS |
| 22 | anti-TROP2 TP3, | VFMSKTYM |
| | HVR-H1 | |
| | polypeptide | |
| 23 | anti-TROP2 TP3, | ISADGKLT |
| | HVR-H2 | |
| | polypeptide | |
| 24 | anti-TROP2 TP3, | WGDKDYDRRD |
| | HVR-H3 | |
| | polypeptide | |
| 25 | anti-TROP2 | QLQLVESGGGLVQPGGSLRLSCAASGFTLDDYAMRWFRQAPGKAREG |
| | #TP4 VHH amino | VSCISRSDGTTYYADSVKGRFTISRNNAENTVYLQMNSLKPEDTAVYYC |
| | acid sequence | AADSLGCNGYSDWGVDYWAKGTLVTVSS |
| 26 | anti-TROP2 TP4, | GFTLDDYA |
| | HVR-H1 | |
| | polypeptide | |
| 27 | anti-TROP2 TP4, | ISRSDGTT |
| | HVR-H2 | |
| | polypeptide | |
| 28 | anti-TROP2 TP4, | AADSLGCNGYSD |
| | HVR-H3 | |
| | polypeptide | |
| 29 | anti-TROP2 | QLQLVESGGGLVQPGGSLKLSCAASGFTLDYWTIGWFRQAPGKEREGV |
| | #TP5 VHH amino | SCISSSDGSTYYADSVKGRFTISRDNAKRTVNLQMNSLKPEDTAVYYCA |
| | acid sequence | AGSLGYCSRSTPQYEYDFWGQGTQVTVSS |
| 30 | anti-TROP2 TP5, | GFTLDYWT |
| | HVR-H1 | |
| | polypeptide | |
| 31 | anti-TROP2 TP5, | ISSSDGST |
| | HVR-H2 | |
| | polypeptide | |
| 32 | anti-TROP2 TP5, | AAGSLGYCSRSTPQYEYDF |
| | HVR-H3 | |
| | polypeptide | |
| 33 | anti-TROP2 | QLQLVESGGGLVQPGGSLRLSCAASGTLDYWTVGWFRQAPGKEREGV |
| | #TP6 VHH amino | SCISSSDSSTYYADSVKGRFTISRDNAKNTVYLQMNSLKPEDTAVYYCA |
| | acid sequence | AGSLGYCSRSTPQYEYDFWGQGTQVTVSS |
| 34 | anti-TROP2 TP6, | GTLDYWTV |
| | HVR-H1 | |
| | polypeptide | |
| 35 | anti-TROP2 TP6, | ISSSDSST |
| | HVR-H2 | |
| | polypeptide | |
| 36 | anti-TROP2 TP6, | AAGSLGYCSRSTPQYEYDF |
| | HVR-H3 | |
| | polypeptide | |
| 37 | anti-TROP2 | QLQLVESGGGLVQPGGSLRLSCAASGFSLEHWTIGWFRQAPGKEREGV |
| | #TP7 VHH amino | SCISSSDASTYYADSVKGRFTISRDNAKKSVYLQMNSLKLEDTAVYYCAA |
| | acid sequence | GSLGYCSRSTPQYEYDYWGQGTQVTVSS |
| 38 | anti-TROP2 TP7, | GFSLEHWT |
| | HVR-H1 | |
| | polypeptide | |
| 39 | anti-TROP2 TP7, | ISSSDAST |
| | HVR-H2 | |
| | polypeptide | |
| 40 | anti-TROP2 TP7, | AAGSLGYCSRSTPQYEYDY |
| | HVR-H3 | |
| | polypeptide | |
| 41 | anti-TROP2 | QLQLVESGGGLVQPGGSLRLSCAASGFTLDYWTIGWFRQAPGKEREGV |
| | #TP8 VHH amino | SCVSSSGGITYYADSVKGRFTISRDNEKRTVYLQMNSLKPEDTAVYYCA |
| | acid sequence | AGSLGYCSRSTPQYEYDYWGQGTQVTVSS |
| 42 | anti-TROP2 TP8, | GFTLDYWT |
| | HVR-H1 | |
| | polypeptide | |
| 43 | anti-TROP2 TP8, | VSSSGGITY |
| | HVR-H2 | |
| | polypeptide | |
| 44 | anti-TROP2 TP8, | AAGSLGYCSRSTPQYEYDY |
| | HVR-H3 | |
| | polypeptide | |
| 45 | anti-TROP2 | QVQLVESGGGLVQPGGSLRLSCAASGFSLDYWAIAWFRQAPGKEREG |
| | #TP9 VHH amino | VSCSGSRGSTHYADSVKGRFTISRDNAKNTVYLQMNSLKPEDAGVYYC |
| | acid sequence | AVGSTLYSLTPYEYDYWGQGTQVTVSS |
| 46 | anti-TROP2 TP9, | GFSLDYWA |
| | HVR-H1 | |
| | polypeptide | |
| 47 | anti-TROP2 TP9, | SGSRGSTH |
| | HVR-H2 | |
| | polypeptide | |
| 48 | anti-TROP2 TP9, | AVGSTLYSLTPYEYDY |
| | HVR-H3 | |
| | polypeptide | |
| 49 | anti-TROP2 | QVQLVESGGGLVQPGGSLRLSCAASGFRLDHWAIGWFRQAPGKEREG |
| | #TP10 VHH | VSCSGSDESTYYADSVKGRFTISRDNAKNTVYLQMNSLKPEDTGVYYCA |
| | amino acid | VGSTWYSLTPYEYDYWGQGTQVTVSS |
| | sequence | |
| 50 | anti-TROP2 | GFRLDHWA |
| | TP10, HVR-H1 | |
| | polypeptide | |
| 51 | anti-TROP2 | SGSDESTY |
| | TP10, HVR-H2 | |
| | polypeptide | |
| 52 | anti-TROP2 | AVGSTWYSLTPYEYDY |
| | TP10, HVR-H2 | |
| | polypeptide | |
| 53 | anti-TROP2 | QVQLVESGGGLVQPGGSLRVSCAASGFRLDYYAIGWFRQAPGKEREG |
| | #TP11 VHH | VSSINYSSGSTVYADSVKGRFTISRGNAKNTVYLQMNSLKPEDTAVYYC |
| | amino acid | AVGTVVTSLAPYEYDYWGQGTQVTVSS |
| | sequence | |
| 54 | anti-TROP2 | GFRLDYYA |
| | TP11, HVR-H1 | |
| | polypeptide | |
| 55 | anti-TROP2 | SSINYSSGSTV |
| | TP11, HVR-H2 | |
| | polypeptide | |
| 56 | anti-TROP2 | AVGTVVTSLAPYEYDY |
| | TP11, HVR-H3 | |
| | polypeptide | |
| 57 | anti-TROP2 | QVQLVESGGGLVQPGGSLRLSCAASRFTLDYYAIGWIRQAPGKEREGV |
| | #TP12 VHH | SCISSSGGSTNYADSVKGRFTISRDNAKNTVYLQMNSLKPEDTGVYYCA |
| | amino acid | ATTYACSGYVPRAYEYDYWGQGTQVTVSS |
| | sequence | |
| 58 | anti-TROP2 | SRFTLDYYA |
| | TP12, HVR-H1 | |
| | polypeptide | |
| 59 | anti-TROP2 | ISSSGGSTN |
| | TP12, HVR-H2 | |
| | polypeptide | |
| 60 | anti-TROP2 | AATTYACSGYVPRAYEYDY |
| | TP12, HVR-H3 | |
| | polypeptide | |
| 61 | anti-TROP2 | EVQLVESGGGLVQPGGSLRLSCALSGFTKDYYGIGWFRQAPGKEREGV |
| | #TP13 VHH | SCISVSGSSTNYADSVKGRFTISRDNAKNTVYLQMNSLKPEDTGVYYCA |
| | amino acid | AAFMGSDYVVPEMSREYDYWGQGTQVTVSS |
| | sequence | |
| 62 | anti-TROP2 | SGFTKDYYG |
| | TP13, HVR-H1 | |
| | polypeptide | |
| 63 | anti-TROP2 | ISVSGSSTN |
| | TP13, HVR-H2 | |
| | polypeptide | |
| 64 | anti-TROP2 | AAAFMGSDYVVPEMSREYDY |
| | TP13, HVR-H3 | |
| | polypeptide | |
| 65 | anti-TROP2 | EVQVVESGGGLVQPGGSLRLSCAVSGFTLDWYAIGWFRQAPGKEHEG |
| | #TP14 VHH | VACIDPSERSTYADPVKGRFTISRDNAKNTVHLQMNSLKPEDTAVYYCA |
| | amino acid | ADTLPCFFYGSMWPPPDNWGQGTQVTVSS |
| | sequence | |
| 66 | anti-TROP2 | GFTLDWYA |
| | TP14, HVR-H1 | |
| | polypeptide | |
| 67 | anti-TROP2 | IDPSERST |
| | TP14, HVR-H2 | |
| | polypeptide | |
| 68 | anti-TROP2 | AADTLPCFFYGSMWPPPDN |
| | TP14, HVR-H3 | |
| | polypeptide | |
| 69 | anti-TROP2 | QVQLVESGGGLVQPGGSLRLSCAVSGDIFSFYSMGWYRQAPGEQRELV |
| | #TP15 VHH | AHITSGGSTHYADSVKGRFTISRDNAKNTVYLQMNSPKPEDTAVYYCNA |
| | amino acid | DLDYWVDSMGPYWGQGTQVTVSS |
| | sequence | |
| 70 | anti-TROP2 | GDIFSFYS |
| | TP15, HVR-H1 | |
| | polypeptide | |
| 71 | anti-TROP2 | ITSGGSTH |
| | TP15, HVR-H2 | |
| | polypeptide | |
| 72 | anti-TROP2 | NADLDYWVDSMGPY |
| | TP15, HVR-H3 | |
| | polypeptide | |
| 73 | anti-TROP2 | QVQLVESGGGLVQPGGSLRLSCAASGFAFSFYVMTWYRQAPGKQREQ |
| | #TP16 VHH | VAAITSYGGANYADSVKGRFTISRDNAKNTVYLQMNALTSEDTAVYSCN |
| | amino acid | AHLGPYDYWGQGTQVTVSS |
| | sequence | |
| 74 | anti-TROP2 | GFAFSFYV |
| | TP16, HVR-H1 | |
| | polypeptide | |
| 75 | anti-TROP2 | ITSYGGAN |
| | TP16, HVR-H2 | |
| | polypeptide | |
| 76 | anti-TROP2 | NAHLGPYDY |
| | TP16, HVR-H3 | |
| | polypeptide | |
| 77 | anti-TROP2 | EVQLVESGGGMVQPGGSLRLSCAFSGLTFSNYAMTWYRQAPGKEREL |
| | #TP17 VHH | VAGTPSPGGTTTYADSVKGRFTISRDNAEDTVYLQMNSLKPEDTAVYYC |
| | amino acid | HARWMWPSDNAGMGYWGKGTLVTVSS |
| | sequence | |
| 78 | anti-TROP2 | GLTFSNYA |
| | TP17, HVR-H1 | |
| | polypeptide | |
| 79 | anti-TROP2 | TPSPGGTTT |
| | TP17, HVR-H2 | |
| | polypeptide | |
| 80 | anti-TROP2 | HARWMWPSDNAGMGY |
| | TP17, HVR-H3 | |
| | polypeptide | |
| 81 | anti-TROP2 | QVQLVESGGGLVQPGGSLRLSCAASSSIFSIAIMGWYRQAPGKQRELVA |
| | #TP18 VHH | TISATDVTHYADSVKGRFTISRDDAWNAVYLQMDSLKAEDTAVYYCNSY |
| | amino acid | LLQQRVPWGQGTQVTVSS |
| | sequence | |
| 82 | anti-TROP2 | SSIFSIAI |
| | TP18, HVR-H1 | |
| | polypeptide | |
| 83 | anti-TROP2 | ISATDVTH |
| | TP18, HVR-H2 | |
| | polypeptide | |
| 84 | anti-TROP2 | NSYLLQQRVP |
| | TP18, HVR-H3 | |
| | polypeptide | |
| 85 | anti-TROP2 | EVQVVESGGGLVQPGGSLRLSCAASRSVFNIAIMGWYRQAPGKQRELV |
| | #TP19 VHH | ASISATDVTSYADSVKGRFTISRDDAWNAVYLQMDSLKSEDTAVYYCNS |
| | amino acid | YLLQQRVPWGQGTQVTVSS |
| | sequence | |
| 86 | anti-TROP2 | RSVFNIAI |
| | TP19, HVR-H1 | |
| | polypeptide | |
| 87 | anti-TROP2 | ISATDVTS |
| | TP19, HVR-H2 | |
| | polypeptide | |
| 88 | anti-TROP2 | NSYLLQQRVP |
| | TP19, HVR-H3 | |
| | polypeptide | |
| 89 | anti-TROP2 | EVQVVESGGGLVQPGGSLRLSCAASGNIKSTAVMGWHRQAPGKEREW |
| | #TP20 VHH | VAIITTGEDRFSGDRLQYADSVKDRFTISRDNAKNTVYLQMNSLKPEDTA |
| | amino acid | VYYCNVDVLYMADGRYARSEFWGQGTQVTVSS |
| | sequence | |
| 90 | anti-TROP2 | GNIKSTAV |
| | TP20, HVR-H1 | |
| | polypeptide | |
| 91 | anti-TROP2 | ITTGEDRFSGDRLQ |
| | TP20, HVR-H2 | |
| | polypeptide | |
| 92 | anti-TROP2 | NVDVLYMADGRYARSEF |
| | TP20, HVR-H3 | |
| | polypeptide | |

### Example 6: Application of TROP2 Binders to Cell Therapy

Anti-TROP2 VHH, when expressed on the immune cell surface as a binder in CAR fusion protein, can bind TROP2-positive tumor cells resulting in activation of immune cells to kill tumor cells.

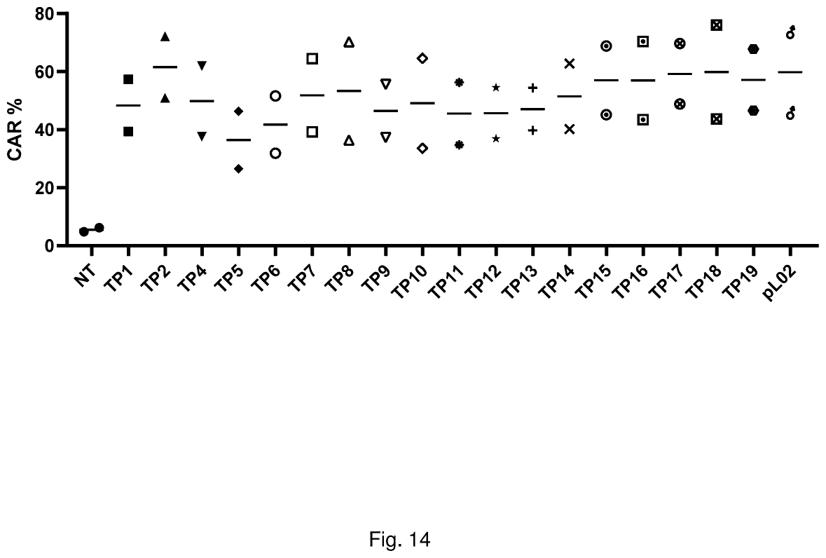

Twenty anti-TROP2 VHH sequences were individually cloned into CAR construct, comprising anti-TROP2 VHH (ID #TP1 to TP20), CD8α hinge (SEQ ID NO: 137) and transmembrane domain (SEQ ID NO: 138), 4-1BB (SEQ ID NO: 139) and CD3Z (SEQ ID NO: 140) signaling domains, and human IL-15 (SEQ ID NO: 131) separated by a T2A sequence (SEQ ID NO: 141) in a gamma-retroviral plasmid (Biovec pharma, Quebec, Canada). Retroviral vectors were generated via transient transfection of CAR vector into the 293vecRDF114 packaging cell line (Biovec pharma, Quebec, Canada) using gene juice (Sigma, St Louis, MO). Retroviral vector supernatant was harvested at 48 and 72 hrs post-transfection and stored at −80° C. until use. Retroviral vectors encoding different TROP2 binder-derived CARs were then used to generate TROP2 CAR γδ T cells. The activated γδ T cells from two healthy donors were efficiently transduced with CAR retroviral vectors as assessed by flow cytometry (FIG. 14).

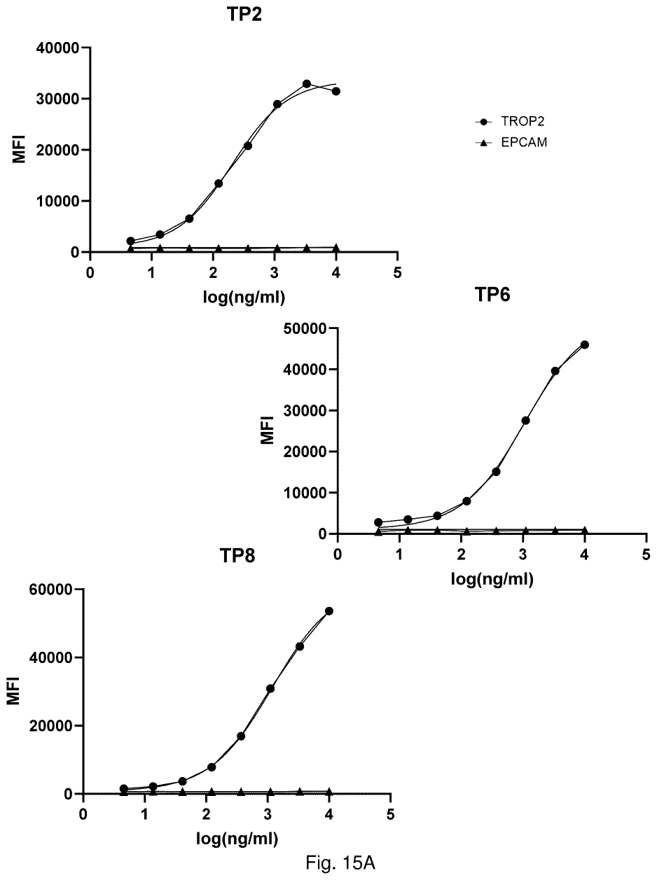

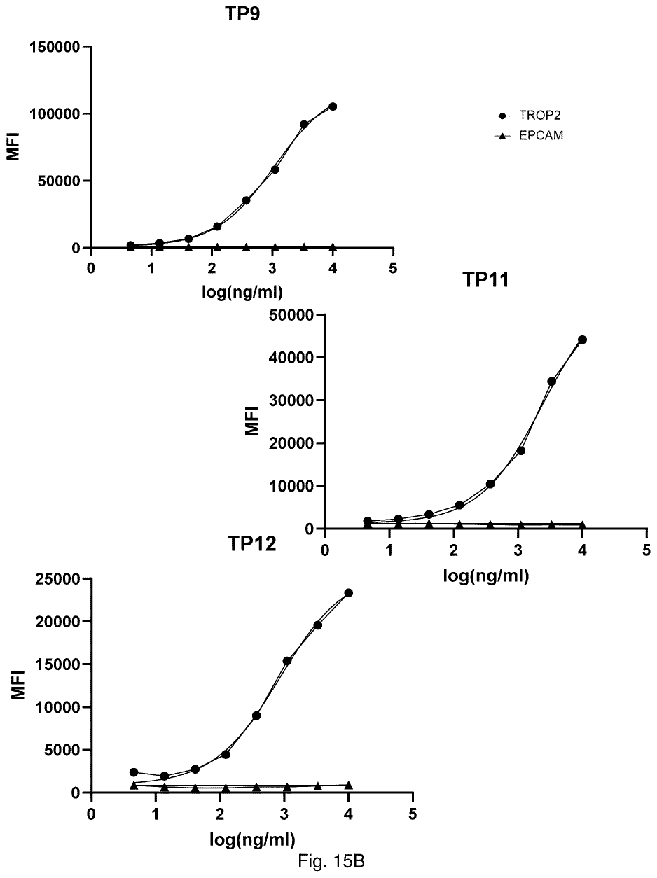

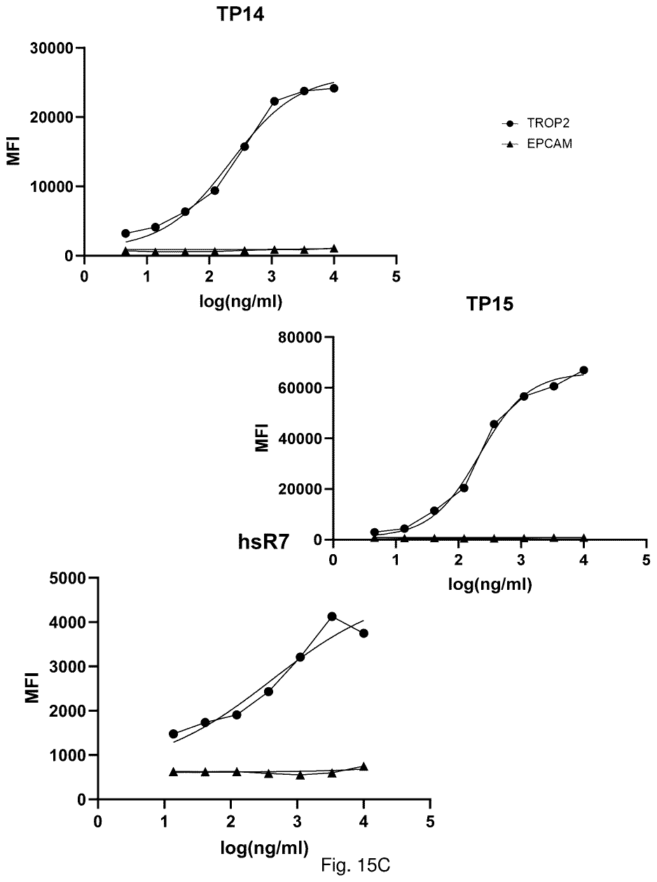

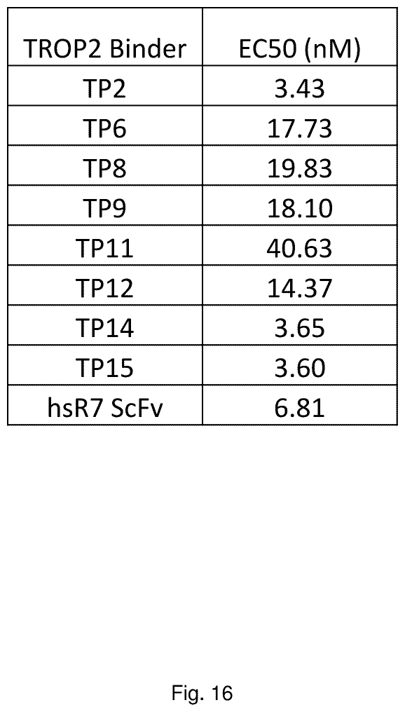

FIGS. 15A-C show examples of displayed TROP2 CAR binders that specifically bind to recombinant human TROP2 protein but not the homolog human EPCAM protein. The binding affinity (EC50) of TROP2 protein against TP2, TP14 or TP15 VHH binder-expressing γδ T cells, as determined by flowcytometry, is in single digit nM, which are similar to γδ T cells expressing a TROP2 scFv binder adopted from a monoclonal antibody (hRS7) (FIG. 16).

### Evaluation of Anti-Tumor Activity of TROP2 CAR γδ T Cells

### Antitumor Cytotoxic Effects of TROP2 CAR γδ T Cells

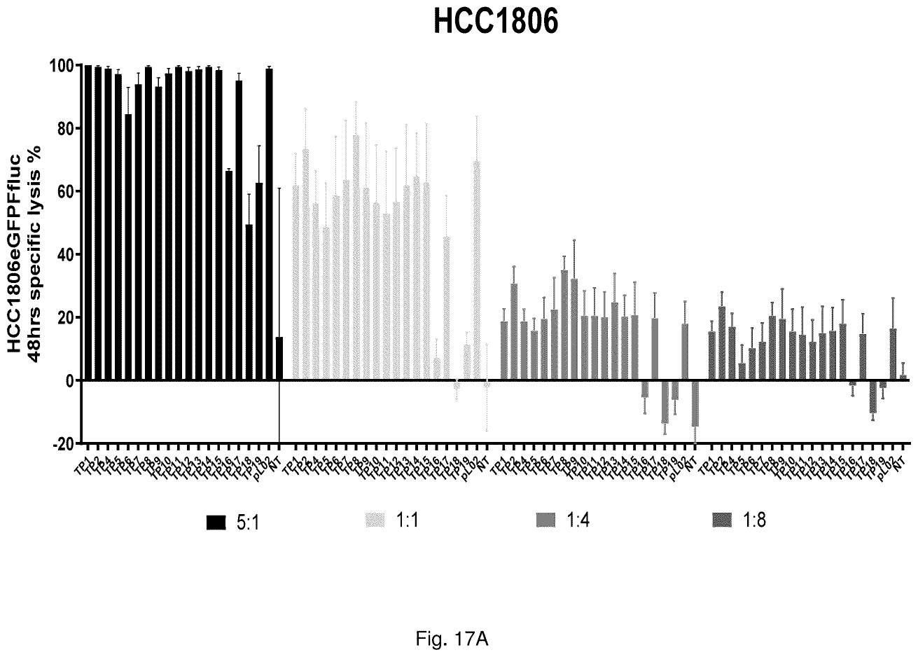

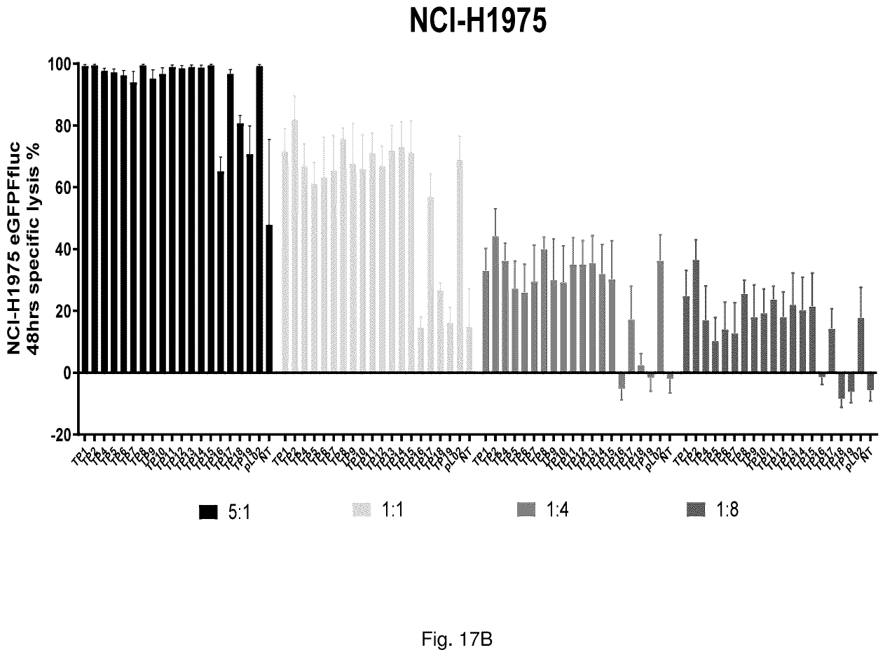

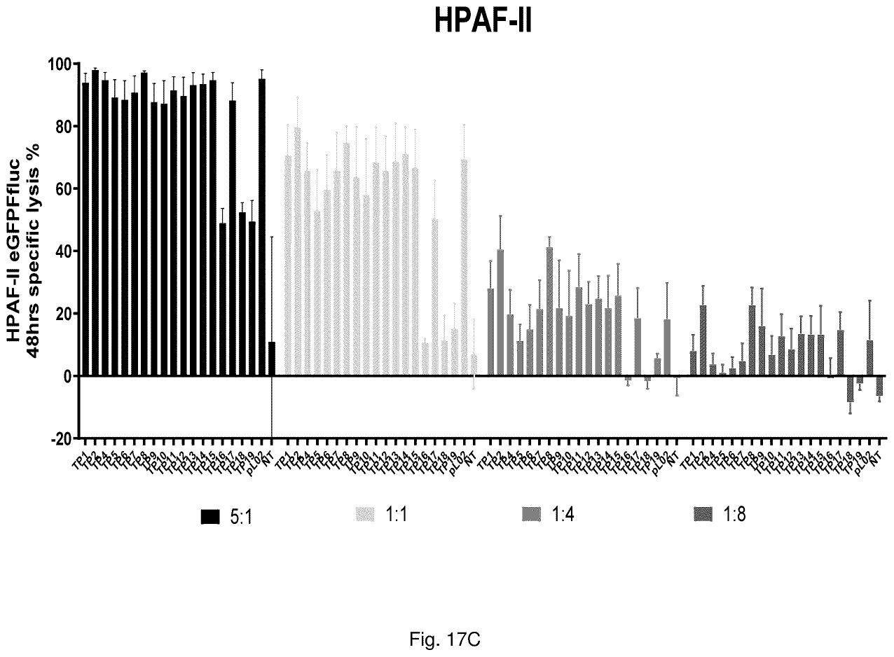

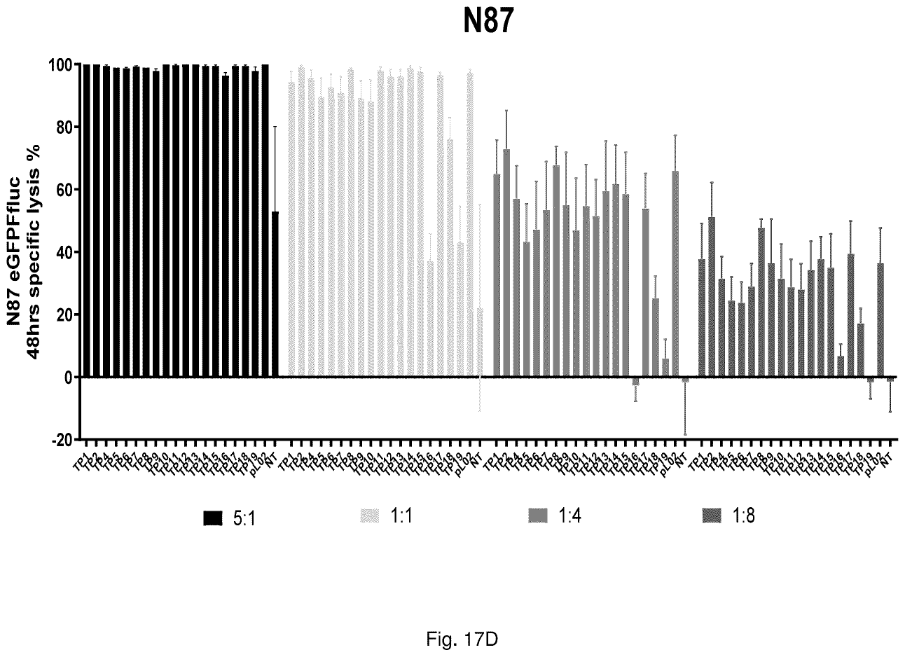

The antitumor cytotoxic activities of the TROP2 CAR γδ T cells bearing TP1-TP20 VHH binders or hRS7 scFv binder (pL02) compared with unmodified γδ T cells (NT), derived from two donors, were assessed against a panel of TROP2-positive tumor cell lines (i.e., HCC1806, NCI-H1975, HPAF-II, and N87). Following 48 hr co-culture with GFP-Luc-expressing respective tumor cell line at the indicated E:T ratio from 5:1 to 1:8, TROP2 CAR γδ T cells, with the exception of TP16, TP18 and TP19, showed significantly more enhanced killing than NT of TROP2-postive TNBC cell lines HCC1806 (FIG. 17A), NSCLC cell line NCI-H1975 (FIG. 17B), PDAC cell line HPAF-II (FIG. 17C) and gastric cancer N87 cell line (FIG. 17D).

### Antitumor Activity of TROP2 CAR γδ T Cells in 3D Tumor Spheroid Model

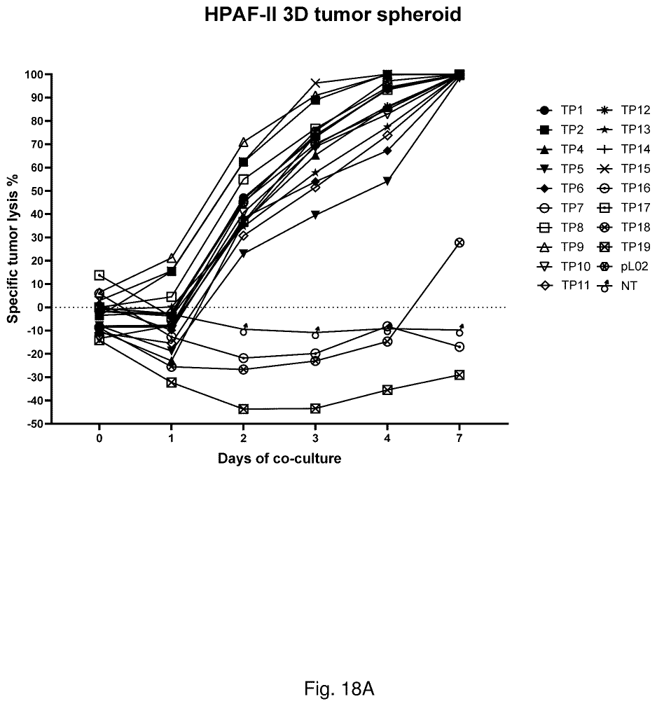

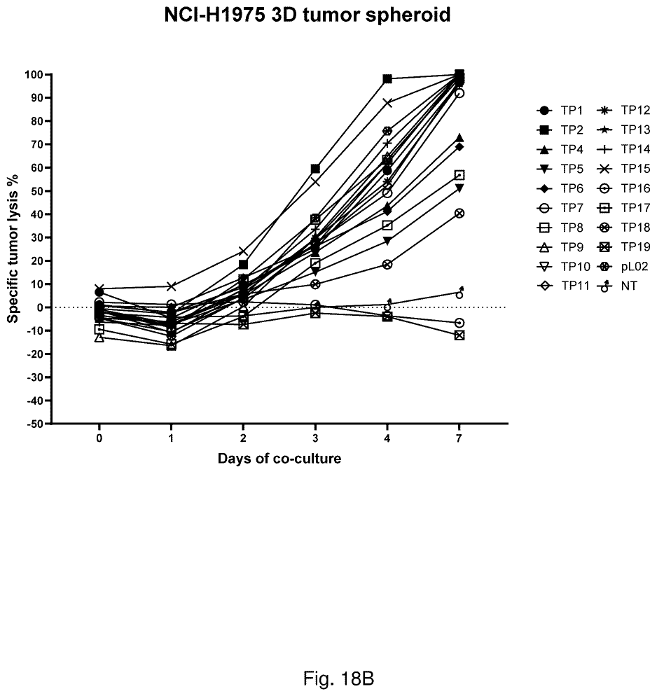

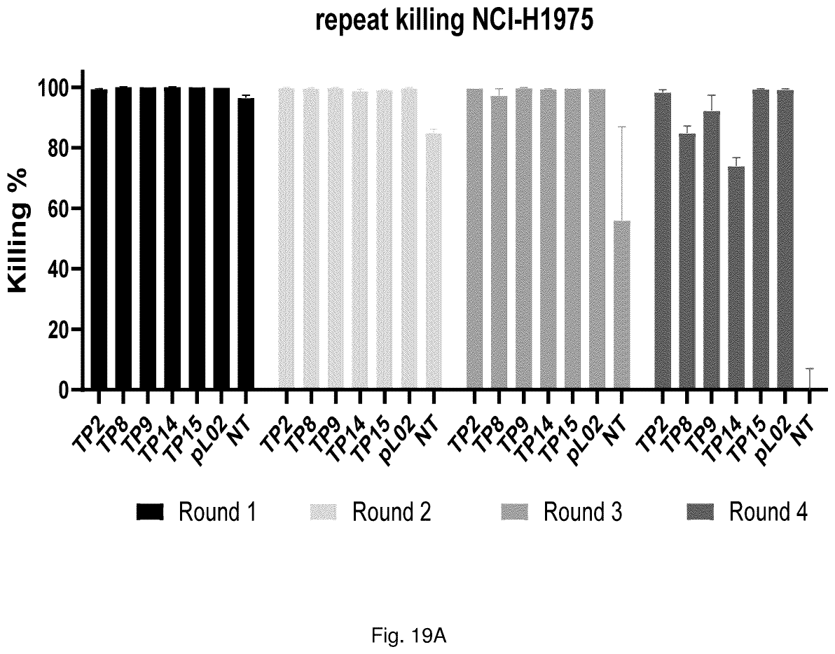

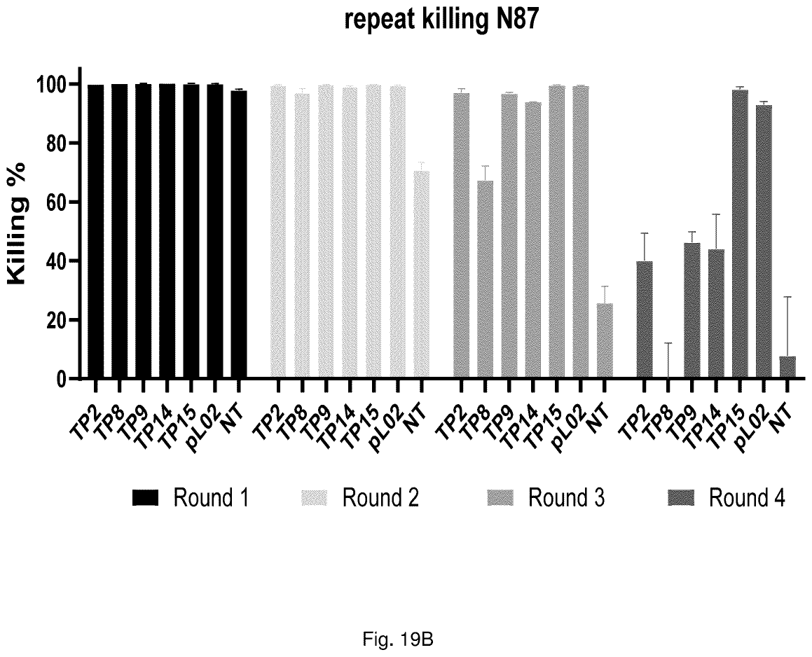

The cytotoxicity of TROP2 CAR γδ T cells again 3D tumor spheroids were also tested with TROP2-positive cancer cell lines HPAF-II and NCI-H1975. Tumor spheroids were seeded with 2,500 cells of GFP-Luc-expressing HPAF-II and NCI-H1975. When the tumor spheroids were established after 3 days, 1,250 TROP2 CAR γδ T cells or NT γδ T cells were added for coculture. Tumor spheroids were monitored, and total green fluorescence intensity was quantified on BIOTEK CYTATION 5 cell imaging multimode reader daily up to 7 days. NT γδ T cells showed limited tumor killing activity, which was dramatically increased in TROP2 CAR γδ T cells, with the exception of TP16, TP18, and TP19. TROP2 CAR γδ T cells effectively infiltrated tumor spheroids and eliminated all detectable tumor cells over 7 days of coculture (FIGS. 18A, 18B).

Serial Killing Capacity of TROP2 CAR γδ T Cells

A subset of TROP2 CAR γδ T cells were further tested against repeat challenges (×4) of TROP2-positive tumor cell lines NCI-H1975, N87, and HPAF-II. This assay measures the serial and persistent tumor killing capabilities of CAR γδ T cells to achieve durable tumor control. On Day 0, duplicate plates of 5,000 GFP-Luc-labeled tumor cells per well were either left untreated (TC-only) or co-cultured with 20,000 TROP2 CAR γδ T cells (TP2, TP8, TP9, TP14, TP15 and pL02) or unmodified γδ T cells (NT). On Days 3, 6, and 9, T cells in one plate were transferred into a new plate that was seeded with 5,000 fresh tumor cells. A duplicate plate was used to quantify tumor-specific killing by luciferase assay. In comparison to NT, TROP2 CAR γδ T cells, with the exception of TP8, sustained significant tumor killing activity after 4 rounds of tumor antigen challenges, resulting in durable tumor control. Table 3 below provides VH, VL, and HVR amino acid sequences for the anti-TROP2 scFv binder of pL02 used in this Example.

TABLE 3
Anti-TROP2 scFv binder sequences

| SEQ ID | | |
|---|---|---|
| NO | Notes | Sequences |
| 93 | anti-TROP2 | QVQLQQSGSELKKPGASVKVSCKASGYTFTNYGMNWVKQAPGQGLKW |
| | #PL02 VH amino | MGWINTYTGEPTYTDDFKGRFAFSLDTSVSTAYLQISSLKADDTAVYFCA |
| | acid sequence | RGGFGSSYWYFDVWGQGSLVTVSS |
| 94 | anti-TROP2 | GYTFTNYG |
| | #PL02, HVR-H1 | |
| | polypeptide | |
| 95 | anti-TROP2 | INTYTGEP |
| | #PL02, HVR-H2 | |
| | polypeptide | |
| 96 | anti-TROP2 | ARGGFGSSYWYFDV |
| | #PL02, HVR-H3 | |
| | polypeptide | |
| 97 | anti-TROP2 | DIQLTQSPSSLSASVGDRVSITCKASQDVSIAVAWYQQKPGKAPKLLIYS |
| | #PL02 VL amino | ASYRYTGVPDRFSGSGSGTDFTLTISSLQPEDFAVYYCQQHYITPLTFGA |
| | acid sequence | GTKVEIK |
| 98 | anti-TROP2 | QDVSIA |
| | #PL02, HVR-L1 | |
| | polypeptide | |
| | anti-TROP2 | SAS |
| | #PL02, HVR-L2 | |
| | polypeptide | |
| 100 | anti-TROP2 | QQHYITPLT |
| | #PL02, HVR-L3 | |
| | polypeptide | |
| 101 | anti-TROP2 | DIQLTQSPSSLSASVGDRVSITCKASQDVSIAVAWYQQKPGKAPKLLIYS |
| | #PL02 scFv (VL- | ASYRYTGVPDRFSGSGSGTDFTLTISSLQPEDFAVYYCQQHYITPLTFGA |
| | linker-VH) amino | GTKVEIKGGGGSGGGGSGGGGSQVQLQQSGSELKKPGASVKVSCKAS |
| | acid sequence | GYTFTNYGMNWVKQAPGQGLKWMGWINTYTGEPTYTDDFKGRFAFSL |
| | | DTSVSTAYLQISSLKADDTAVYFCARGGFGSSYWYFDVWGQGSLVTVS |
| | | S |

### Example 7: Generation and Evaluation of Bispecific CAR Targeting TROP2 and PD-L1 Generation and Expansion of Bispecific CAR

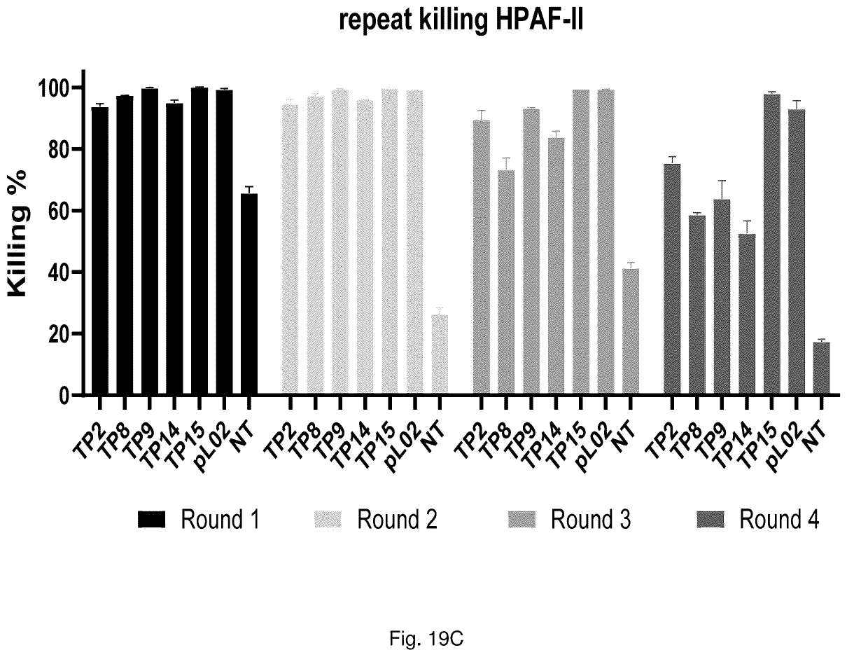

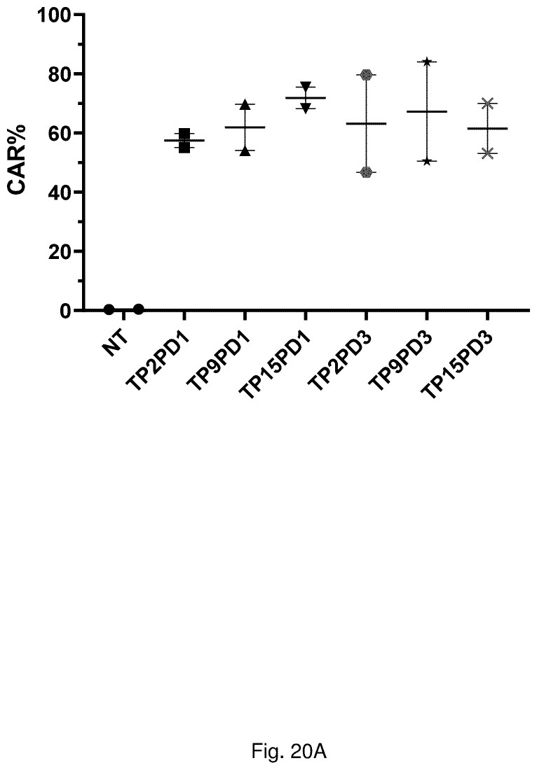

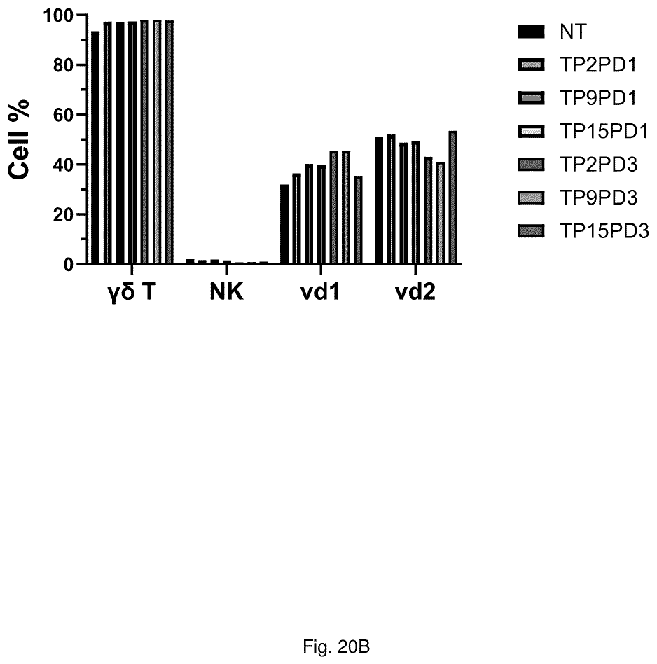

TROP2 and PD-L1 dual targeting CAR can be constructed by tandemly linking one or more TROP2 binder sequences with one or more PD-L1 binder sequences in either direction. Table 4 shows the examples of dual CAR comprising selected TROP2 binders TP2, TP9, or TP15 and PD-L1 binders PD1 or PD3. Other example constructs include TP2-TP15-PD1, TP15-TP2-PD1, TP2-TP15-PD3, TP15-TP2-PD3, TP2-TP15-PD1-PD3, TP15-TP2-PD1-PD3, TP2-TP15-PD3-PD1, TP15-TP2-PD3-PD1, and other configurations that include TP2, TP9, TP15, PD1, PD3, and/or any other TROP2 and/or PD-L1 VHH polypeptide provided herein in any order. The sequences in Table 4 below were individually cloned into CAR cretroviral plasmid (Biovec pharma, Quebec, Canada). Retroviral vectors were generated via transient transfection of CAR plasmid into the 293vecRDF114 packaging cell line (Biovec pharma, Quebec, Canada). Retroviral vector supernatant was then used to generate TROP2-PD-L1 dual CAR γδ T cells. The activated γδ T cells from two healthy 10 donors were efficiently transduced with dual CAR retroviral vectors in the range of 50%-80% as assessed by flow cytometry (FIG. 20A). The expanded γδ T cells following a 14-day culture were composed of 90-95% γδ T cells (CD3+γδTCR+) and 1-3% of NK cells (CD3-CD56+). The γδ T cells included subtypes Vδ1 (CD3+Vδ1TCR+) T cells and Vδ2 (CD3+Vδ2TCR+) T cells at approximately 40% and 50%, respectively (FIG. 20B).

TABLE 4
Bispecific CAR sequences

| SEQ ID | | |
|---|---|---|
| NO | Notes | Sequences |
| 102 | TROP2 & PD-L1 | EVQLVESGGGLAQPGGSLRLSCAASGFTFSTYAMSWARQAPGKGLEW |
| | Dual CAR | VSGIYADGSGTYYADSVKGRFTISRDNAKNTLYLQMNSLKPEDTALYYCA |
| | #TP2PD1 (VH- | QGYSDYDRRLGEYDYWGQGTQVTVSSGGGGSGGGGSGGGGSDVQL |
| | Linker-VH) | QESGGGLVQAGGSLRLSCAASESIASINVMGWYRQAPGKQRELVAIITN |
| | amino acid | RGTTSYPDSVKGRFTISRDNGKNTVYLQMNTLKPEDTAVYYCNAPWTD |
| | sequence | GINYWGKGTQVTVSS |
| 103 | TROP2 & PD-L1 | QVQLVESGGGLVQPGGSLRLSCAASGFSLDYWAIAWFRQAPGKEREGV |
| | Dual CAR | SCSGSRGSTHYADSVKGRFTISRDNAKNTVYLQMNSLKPEDAGVYYCA |
| | #TP9PD1 (VH- | VGSTLYSLTPYEYDYWGQGTQVTVSSGGGGSGGGGSGGGGSDVQLQ |
| | Linker-VH) | ESGGGLVQAGGSLRLSCAASESIASINVMGWYRQAPGKQRELVAIITNR |
| | amino acid | GTTSYPDSVKGRFTISRDNGKNTVYLQMNTLKPEDTAVYYCNAPWTDGI |
| | sequence | NYWGKGTQVTVSS |
| 104 | TROP2 & PD-L1 | QVQLVESGGGLVQPGGSLRLSCAVSGDIFSFYSMGWYRQAPGEQRELV |
| | Dual CAR | AHITSGGSTHYADSVKGRFTISRDNAKNTVYLQMNSPKPEDTAVYYCNA |
| | #TP15PD1 (VH- | DLDYWVDSMGPYWGQGTQVTVSSGGGGSGGGGSGGGGSDVQLQES |
| | Linker-VH) | GGGLVQAGGSLRLSCAASESIASINVMGWYRQAPGKQRELVAIITNRGT |
| | amino acid | TSYPDSVKGRFTISRDNGKNTVYLQMNTLKPEDTAVYYCNAPWTDGINY |
| | sequence | WGKGTQVTVSS |
| 105 | TROP2 & PD-L1 | EVQLVESGGGLAQPGGSLRLSCAASGFTFSTYAMSWARQAPGKGLEW |
| | Dual CAR | VSGIYADGSGTYYADSVKGRFTISRDNAKNTLYLQMNSLKPEDTALYYCA |
| | #TP2PD3 (VH- | QGYSDYDRRLGEYDYWGQGTQVTVSSGGGGSGGGGSGGGGSDVQL |
| | Linker-VH) | QESGGGLVQAGDSLGLSCAASGRAFSLFEMAWYRQAPGKAREFVAAIM |
| | amino acid | WRGGHTVYADSVKGRSTISSDNTKNTVYLQMNSLKPEDTAIYYCRGKVV |
| | sequence | GSDYWGQGTQVTVSS |
| 106 | TROP2 & PD-L1 | QVQLVESGGGLVQPGGSLRLSCAASGFSLDYWAIAWFRQAPGKEREGV |
| | Dual CAR | SCSGSRGSTHYADSVKGRFTISRDNAKNTVYLQMNSLKPEDAGVYYCA |
| | #TP9PD3 (VH- | VGSTLYSLTPYEYDYWGQGTQVTVSSGGGGSGGGGSGGGGSDVQLQ |
| | Linker-VH) | ESGGGLVQAGDSLGLSCAASGRAFSLFEMAWYRQAPGKAREFVAAIM |
| | amino acid | WRGGHTVYADSVKGRSTISSDNTKNTVYLQMNSLKPEDTAIYYCRGKVV |
| | sequence | GSDYWGQGTQVTVSS |
| 107 | TROP2 & PD-L1 | QVQLVESGGGLVQPGGSLRLSCAVSGDIFSFYSMGWYRQAPGEQRELV |
| | Dual CAR | AHITSGGSTHYADSVKGRFTISRDNAKNTVYLQMNSPKPEDTAVYYCNA |
| | #TP15PD3 (VH- | DLDYWVDSMGPYWGQGTQVTVSSGGGGSGGGGSGGGGSDVQLQES |
| | Linker-VH) | GGGLVQAGDSLGLSCAASGRAFSLFEMAWYRQAPGKAREFVAAIMWR |
| | amino acid | GGHTVYADSVKGRSTISSDNTKNTVYLQMNSLKPEDTAIYYCRGKVVGS |
| | sequence | DYWGQGTQVTVSS |

### Antitumor Cytotoxic Effects of TROP2-PD-L1 Dual CAR γδ T Cells

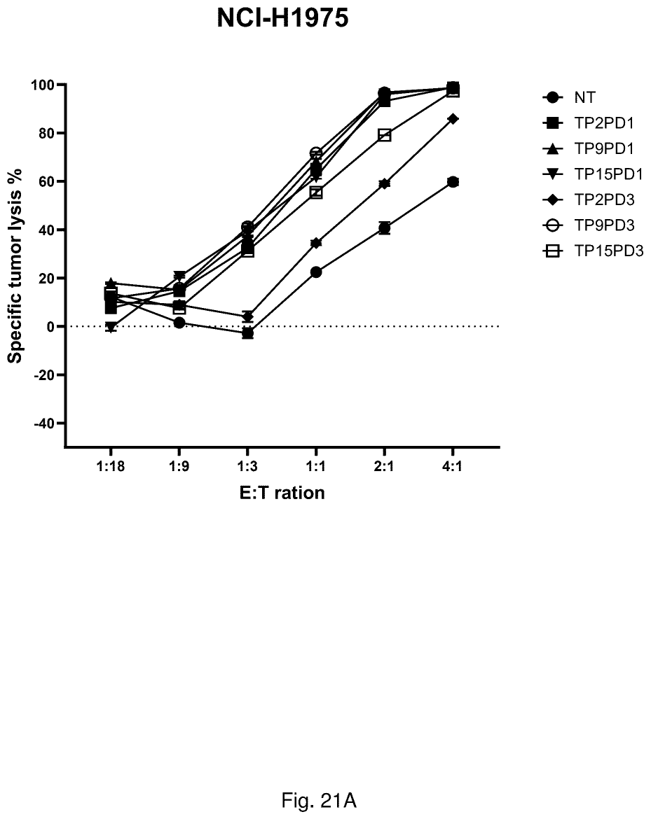

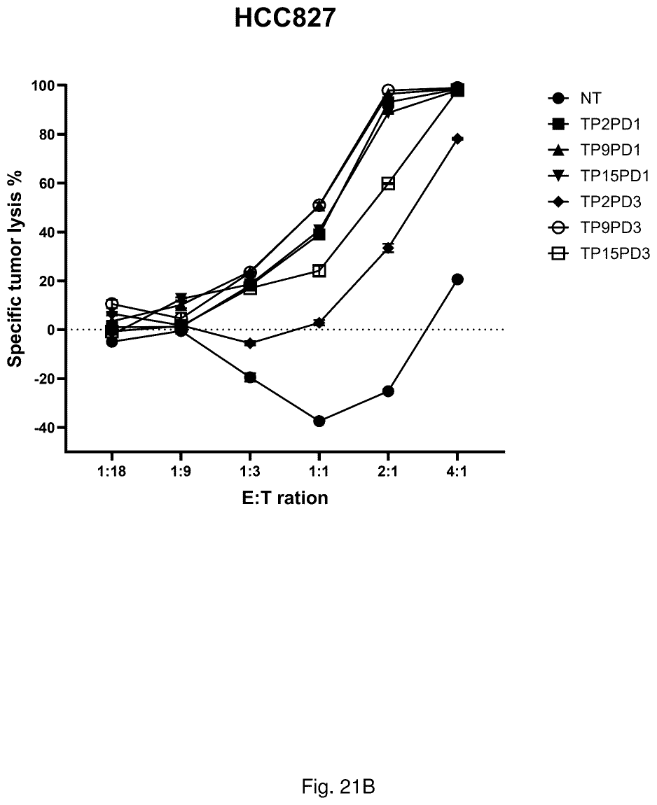

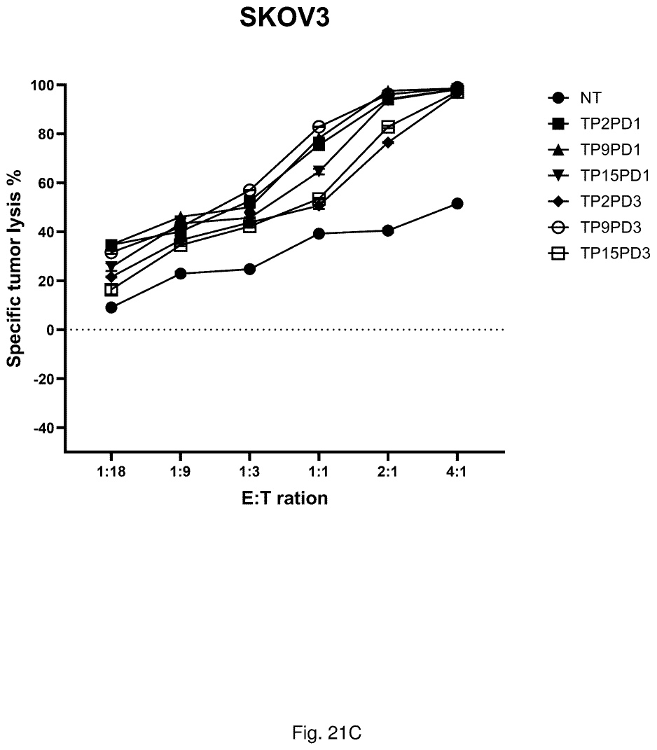

The antitumor cytotoxic activities of the TROP2-PD-L1 dual CAR γδ T cells, compared with unmodified γδ T cells (NT), were assessed in the co-culture with TROP2- and PD-L1-positive tumor cell lines (NCI-H1975, HCC827, and SKOV3) at an increasing E:T ratio from 1:18 to 4:1. Tumor-specific lysis was determined after 48 hr of co-culture by luciferase assay. TROP2-PD-L1 CAR γδ T cells showed potent cytotoxicity against TROP2+PD-L1+tumor cells, achieving approximately 50% of tumor lysis (ED50) at an E:T ratio of 1:1 (in HCC827) or 1:3 (in NCI-H1975 and SKOV3). In the absence of IL-15 supplement, NT γδ T cells also demonstrate innate cytotoxicity towards tumor cells, albeit less potent than TROP2-PD-L1 CAR γδ T cells, requiring higher E:T ratio of >4:1 (in NCI-H1975 and SKOV3) to reach 50% of tumor lysis. (FIGS. 21A-C).

### Antitumor Activity of TROP2-PD-L1 Dual CAR γδ T Cells in 3D Tumor Spheroid Model

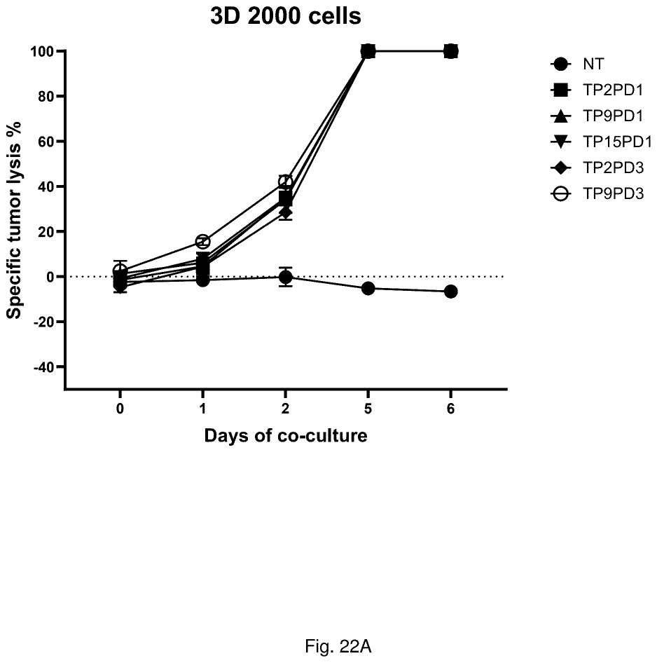

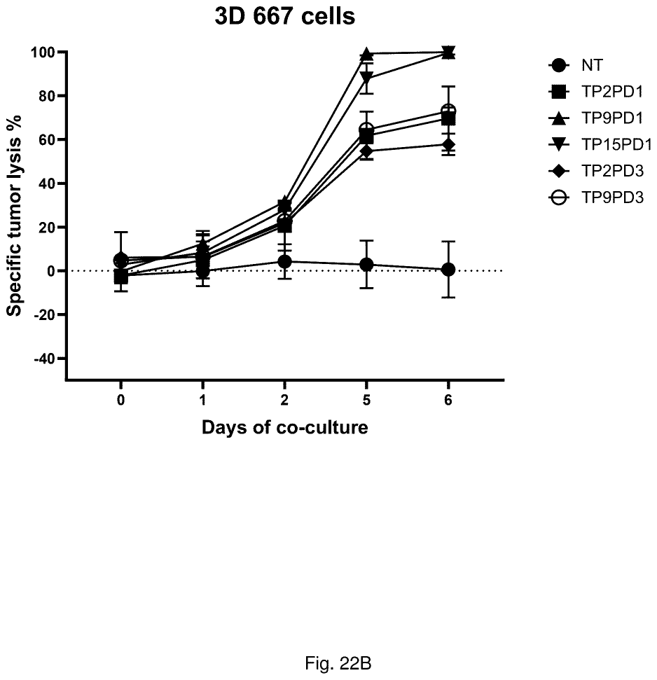

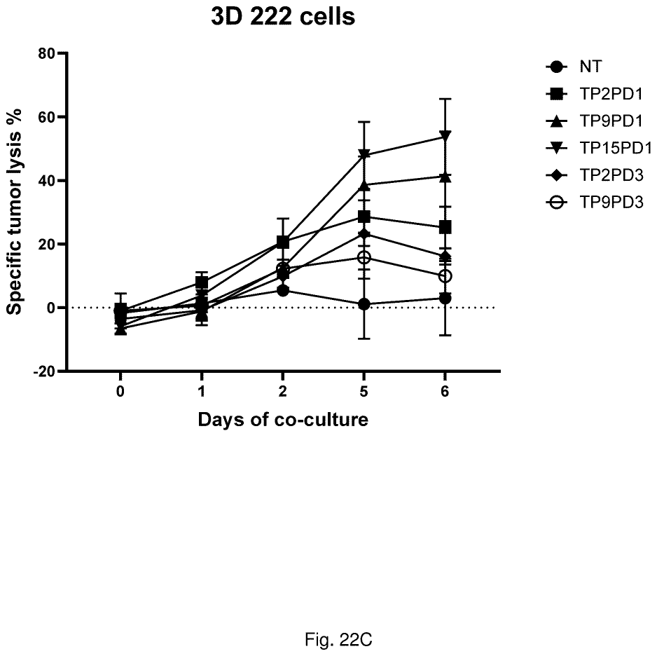

Tumor spheroids were seeded with 5,000 cells of GFP-Luc-expressing NSCLC cell line NCI-H1975. When tumor spheroids were established after 3 days, 2,000, or 667, or 222 TROP2-PD-L1 CAR γδ T cells, or NT γδ T cells were added for coculture. Tumor spheroids were monitored, and total green fluorescence intensity was quantified on BIOTEK CYTATION 5 cell imaging multimode reader (Agilent, Santa Clara, CA) daily up to 6 days. While NT γδ T cells showed no appreciable tumor killing, TROP2-PD-L1 CAR γδ T cells, at ET ratio of approximately 1:2.5, eliminated all detectable tumor cells by Day 5 (FIG. 22A). TROP2-PD-L1 CAR γδ T cells expressing TP15-PD1 binders achieved substantial tumor killing at even lower E:T ratio of approximately 1:7.5 and 1:22.5 (FIGS. 22B and 22C).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents are based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”). Certain implementations of the technology are set forth in the claim(s) that follow(s).

1-20. (canceled)

21. A modified gamma delta T cell comprising a chimeric antigen receptor (CAR), wherein the CAR comprises an antigen binding domain with binding specificity to TROP2 and an antigen binding domain with binding specificity to PD-L1.

22. The modified gamma delta T cell of claim 21, wherein the antigen binding domain with binding specificity to TROP2 comprises a variable heavy domain of heavy chain (VHH) comprising a hypervariable region H1 (HVR-H1) polypeptide, a hypervariable region H2 (HVR-H2) polypeptide, and a hypervariable region H3 (HVR-H3) polypeptide, wherein:
the HVR-H1 polypeptide comprises the amino acid sequence of SEQ ID NO: 18,
the HVR-H2 polypeptide comprises the amino acid sequence of SEQ ID NO: 19, and
the HVR-H3 polypeptide comprises the amino acid sequence of SEQ ID NO: 20; or
the HVR-H1 polypeptide comprises the amino acid sequence of SEQ ID NO: 70,
the HVR-H2 polypeptide comprises the amino acid sequence of SEQ ID NO: 71, and
the HVR-H3 polypeptide comprises the amino acid sequence of SEQ ID NO: 72.

23. The modified gamma delta T cell of claim 22, wherein the VHH comprises a polypeptide that is about 90% or more identical to a polypeptide comprising the amino acid sequence of SEQ ID NO: 17 or SEQ ID NO: 69.

24. The modified gamma delta T cell of claim 21, wherein the antigen binding domain with binding specificity to PD-L1 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 2,
the HVR-H2 polypeptide comprises the amino acid sequence of SEQ ID NO: 3, and
the HVR-H3 polypeptide comprises the amino acid sequence of SEQ ID NO: 4; or
the HVR-H1 polypeptide comprises the amino acid sequence of SEQ ID NO: 6,
the HVR-H2 polypeptide comprises the amino acid sequence of SEQ ID NO: 7, and
the HVR-H3 polypeptide comprises the amino acid sequence of SEQ ID NO: 8.

25. The modified gamma delta T cell of claim 24, wherein the VHH comprises a polypeptide that is about 90% or more identical to a polypeptide comprising the amino acid sequence of SEQ ID NO: 1 or SEQ ID NO: 5.

26. The modified gamma delta T cell of claim 21, wherein the CAR comprises two antigen binding domains with binding specificity to TROP2 and an antigen binding domain with binding specificity to PD-L1.

27. The modified gamma delta T cell of claim 26, wherein the CAR comprises a first antigen binding domain with binding specificity to TROP2 and a second antigen binding domain with binding specificity to TROP2, wherein:
a) the first antigen binding domain comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 18,
the HVR-H2 polypeptide comprises the amino acid sequence of SEQ ID NO: 19, and
the HVR-H3 polypeptide comprises the amino acid sequence of SEQ ID NO: 20; and
b) the second antigen binding domain comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 70,
the HVR-H2 polypeptide comprises the amino acid sequence of SEQ ID NO: 71, and
the HVR-H3 polypeptide comprises the amino acid sequence of SEQ ID NO: 72.

28. The modified gamma delta T cell of claim 27, wherein:
the first antigen binding domain comprises a VHH comprising a polypeptide that is about 90% or more identical to a polypeptide comprising the amino acid sequence of SEQ ID NO: 17; and
the second antigen binding domain comprises a VHH comprising a polypeptide that is about 90% or more identical to a polypeptide comprising the amino acid sequence of SEQ ID NO: 69.

29. The modified gamma delta T cell of claim 26, wherein the antigen binding domain with binding specificity to PD-L1 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 2,
the HVR-H2 polypeptide comprises the amino acid sequence of SEQ ID NO: 3, and
the HVR-H3 polypeptide comprises the amino acid sequence of SEQ ID NO: 4; or
the HVR-H1 polypeptide comprises the amino acid sequence of SEQ ID NO: 6,
the HVR-H2 polypeptide comprises the amino acid sequence of SEQ ID NO: 7, and
the HVR-H3 polypeptide comprises the amino acid sequence of SEQ ID NO: 8.

30. The modified gamma delta T cell of claim 29, wherein the VHH comprises a polypeptide that is about 90% or more identical to a polypeptide comprising the amino acid sequence of SEQ ID NO: 1 or SEQ ID NO: 5.

31. The modified gamma delta T cell of claim 26, wherein the CAR comprises a first antigen binding domain with binding specificity to TROP2, a second antigen binding domain with binding specificity to TROP2, and an antigen binding domain with binding specificity to PD-L1 wherein:
a) the first antigen binding domain with binding specificity to TROP2 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 18,
the HVR-H2 polypeptide comprises the amino acid sequence of SEQ ID NO: 19, and
the HVR-H3 polypeptide comprises the amino acid sequence of SEQ ID NO: 20;
b) the second antigen binding domain with binding specificity to TROP2 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 70,
the HVR-H2 polypeptide comprises the amino acid sequence of SEQ ID NO: 71, and
the HVR-H3 polypeptide comprises the amino acid sequence of SEQ ID NO: 72; and
c) the antigen binding domain with binding specificity to PD-L1 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 2,
the HVR-H2 polypeptide comprises the amino acid sequence of SEQ ID NO: 3, and
the HVR-H3 polypeptide comprises the amino acid sequence of SEQ ID NO: 4.

32. The modified gamma delta T cell of claim 31, wherein:
the first antigen binding domain with binding specificity to TROP2 comprises a VHH comprising a polypeptide that is about 90% or more identical to a polypeptide comprising the amino acid sequence of SEQ ID NO: 17;
the second antigen binding domain with binding specificity to TROP2 comprises a VHH comprising a polypeptide that is about 90% or more identical to a polypeptide comprising the amino acid sequence of SEQ ID NO: 69; and
the antigen binding domain with binding specificity to PD-L1 comprises a VHH comprising a polypeptide that is about 90% or more identical to a polypeptide comprising the amino acid sequence of SEQ ID NO: 1.

33. The modified gamma delta T cell of claim 26, wherein the CAR comprises a first antigen binding domain with binding specificity to TROP2, a second antigen binding domain with binding specificity to TROP2, and an antigen binding domain with binding specificity to PD-L1 wherein:
a) the first antigen binding domain with binding specificity to TROP2 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 18,
the HVR-H2 polypeptide comprises the amino acid sequence of SEQ ID NO: 19, and
the HVR-H3 polypeptide comprises the amino acid sequence of SEQ ID NO: 20;
b) the second antigen binding domain with binding specificity to TROP2 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 70,
the HVR-H2 polypeptide comprises the amino acid sequence of SEQ ID NO: 71, and
the HVR-H3 polypeptide comprises the amino acid sequence of SEQ ID NO: 72; and
c) the antigen binding domain with binding specificity to PD-L1 comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 6,
the HVR-H2 polypeptide comprises the amino acid sequence of SEQ ID NO: 7, and
the HVR-H3 polypeptide comprises the amino acid sequence of SEQ ID NO: 8.

34. The modified gamma delta T cell of claim 33, wherein:
the first antigen binding domain with binding specificity to TROP2 comprises a VHH comprising a polypeptide that is about 90% or more identical to a polypeptide comprising the amino acid sequence of SEQ ID NO: 17;
the second antigen binding domain with binding specificity to TROP2 comprises a VHH comprising a polypeptide that is about 90% or more identical to a polypeptide comprising the amino acid sequence of SEQ ID NO: 69; and
the antigen binding domain with binding specificity to PD-L1 comprises a VHH comprising a polypeptide that is about 90% or more identical to a polypeptide comprising the amino acid sequence of SEQ ID NO: 5.

35. The modified gamma delta T cell of claim 21, wherein the CAR comprises two antigen binding domains with binding specificity to TROP2 and two antigen binding domains with binding specificity to PD-L1.

36. The modified gamma delta T cell of claim 35, wherein the CAR comprises a first antigen binding domain with binding specificity to TROP2 and a second antigen binding domain with binding specificity to TROP2, wherein:
a) the first antigen binding domain comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 18,
the HVR-H2 polypeptide comprises the amino acid sequence of SEQ ID NO: 19, and
the HVR-H3 polypeptide comprises the amino acid sequence of SEQ ID NO: 20; and
b) the second antigen binding domain comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 70,
the HVR-H2 polypeptide comprises the amino acid sequence of SEQ ID NO: 71, and
the HVR-H3 polypeptide comprises the amino acid sequence of SEQ ID NO: 72.

37. The modified gamma delta T cell of claim 35, wherein the CAR comprises a first antigen binding domain with binding specificity to PD-L1 and a second antigen binding domain with binding specificity to PD-L1, wherein:
a) the first antigen binding domain comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 2,
the HVR-H2 polypeptide comprises the amino acid sequence of SEQ ID NO: 3, and
the HVR-H3 polypeptide comprises the amino acid sequence of SEQ ID NO: 4; and
b) the second antigen binding domain comprises a VHH comprising an HVR-H1 polypeptide, an HVR-H2 polypeptide, and an HVR-H3 polypeptide, wherein:
the HVR-H1 polypeptide comprises the amino acid sequence of SEQ ID NO: 6,
the HVR-H2 polypeptide comprises the amino acid sequence of SEQ ID NO: 7, and
the HVR-H3 polypeptide comprises the amino acid sequence of SEQ ID NO: 8.

38. The modified gamma delta T cell of claim 21, wherein the CAR comprises an antigen binding domain with binding specificity to TROP2 and two antigen binding domains with binding specificity to PD-L1.

39. The modified gamma delta T cell of claim 21, wherein the CAR further comprises an IL-15 component.

40. The modified gamma delta T cell of claim 21, further comprising a genome modification, wherein the genome modification is a disrupted gene encoding TGF-beta 2 receptor.


SEQUENCE LISTING (142 sequences)

SEQ ID NO: 1 - protein, 116 aa, synthetic construct
DVQLQESGGGLVQAGGSLRLSCAASESIASINVMGWYRQAPGKQRELVAIITNRGTTSYPDSVKGRFTISRDNGKNTVYLQMNTLKPEDTAVYYCNAPWTDGINYWGKGTQVTVSS

SEQ ID NO: 2 - protein, 8 aa, synthetic construct
ESIASINV

SEQ ID NO: 3 - protein, 7 aa, synthetic construct
ITNRGTT

SEQ ID NO: 4 - protein, 10 aa, synthetic construct
NAPWTDGINY

SEQ ID NO: 5 - protein, 115 aa, synthetic construct
DVQLQESGGGLVEPGGSLRLSCAASGFTFNHYGMSWVRQAPGKGLQWVSTLFTTGSSQYANSVKGRFTISRDAAENTLYLHMNNLKPEDTAVYFCAKGVGLGSERGPGTQVTVSS

SEQ ID NO: 6 - protein, 8 aa, synthetic construct
GFTFNHYG

SEQ ID NO: 7 - protein, 7 aa, synthetic construct
LFTTGSS

SEQ ID NO: 8 - protein, 9 aa, synthetic construct
AKGVGLGSE

SEQ ID NO: 9 - protein, 116 aa, synthetic construct
DVQLQESGGGLVQAGDSLGLSCAASGRAFSLFEMAWYRQAPGKAREFVAAIMWRGGHTVYADSVKGRSTISSDNTKNTVYLQMNSLKPEDTAIYYCRGKVVGSDYWGQGTQVTVSS

SEQ ID NO: 10 - protein, 8 aa, synthetic construct
GRAFSLFE

SEQ ID NO: 11 - protein, 8 aa, synthetic construct
IMWRGGHT

SEQ ID NO: 12 - protein, 9 aa, synthetic construct
RGKVVGSDY

SEQ ID NO: 13 - protein, 123 aa, synthetic construct
EVQVVESGGGLAQPGGSLRLSCAASGFWFSTYAMSWARQAPGKGLEWVSGIYADGSATYYADSVKGRFTISRDNAKNTLYLHMNSLKPEDTALYYCAQGYSDYDRRVGGYDYWGQGTQVTVSS

SEQ ID NO: 14 - protein, 8 aa, synthetic construct
GFWFSTYA

SEQ ID NO: 15 - protein, 8 aa, synthetic construct
IYADGSAT

SEQ ID NO: 16 - protein, 16 aa, synthetic construct
AQGYSDYDRRVGGYDY

SEQ ID NO: 17 - protein, 123 aa, synthetic construct
EVQLVESGGGLAQPGGSLRLSCAASGFTFSTYAMSWARQAPGKGLEWVSGIYADGSGTYYADSVKGRFTISRDNAKNTLYLQMNSLKPEDTALYYCAQGYSDYDRRLGEYDYWGQGTQVTVSS

SEQ ID NO: 18 - protein, 8 aa, synthetic construct
GFTFSTYA

SEQ ID NO: 19 - protein, 8 aa, synthetic construct
IYADGSGT

SEQ ID NO: 20 - protein, 16 aa, synthetic construct
AQGYSDYDRRLGEYDY

SEQ ID NO: 21 - protein, 118 aa, synthetic construct
QVQLVESGGGLAQPGGSLRLSCATSVFMSKTYMMSWYRQAPGKGLEWVADISADGKLTYYDDAEEGFFLFSEDDHKNTVLLMMNLLKHDSTILRCCAWGDKDYDRRDWGHDNWVTVSS

SEQ ID NO: 22 - protein, 8 aa, synthetic construct
VFMSKTYM

SEQ ID NO: 23 - protein, 8 aa, synthetic construct
ISADGKLT

SEQ ID NO: 24 - protein, 10 aa, synthetic construct
WGDKDYDRRD

SEQ ID NO: 25 - protein, 124 aa, synthetic construct
QLQLVESGGGLVQPGGSLRLSCAASGFTLDDYAMRWFRQAPGKAREGVSCISRSDGTTYYADSVKGRFTISRNNAENTVYLQMNSLKPEDTAVYYCAADSLGCNGYSDWGVDYWAKGTLVTVSS

SEQ ID NO: 26 - protein, 8 aa, synthetic construct
GFTLDDYA

SEQ ID NO: 27 - protein, 8 aa, synthetic construct
ISRSDGTT

SEQ ID NO: 28 - protein, 12 aa, synthetic construct
AADSLGCNGYSD

SEQ ID NO: 29 - protein, 126 aa, synthetic construct
QLQLVESGGGLVQPGGSLKLSCAASGFTLDYWTIGWFRQAPGKEREGVSCISSSDGSTYYADSVKGRFTISRDNAKRTVNLQMNSLKPEDTAVYYCAAGSLGYCSRSTPQYEYDFWGQGTQVTVSS

SEQ ID NO: 30 - protein, 8 aa, synthetic construct
GFTLDYWT

SEQ ID NO: 31 - protein, 8 aa, synthetic construct
ISSSDGST

SEQ ID NO: 32 - protein, 19 aa, synthetic construct
AAGSLGYCSRSTPQYEYDF

SEQ ID NO: 33 - protein, 125 aa, synthetic construct
QLQLVESGGGLVQPGGSLRLSCAASGTLDYWTVGWFRQAPGKEREGVSCISSSDSSTYYADSVKGRFTISRDNAKNTVYLQMNSLKPEDTAVYYCAAGSLGYCSRSTPQYEYDFWGQGTQVTVSS

SEQ ID NO: 34 - protein, 8 aa, synthetic construct
GTLDYWTV

SEQ ID NO: 35 - protein, 8 aa, synthetic construct
ISSSDSST

SEQ ID NO: 36 - protein, 19 aa, synthetic construct
AAGSLGYCSRSTPQYEYDF

SEQ ID NO: 37 - protein, 126 aa, synthetic construct
QLQLVESGGGLVQPGGSLRLSCAASGFSLEHWTIGWFRQAPGKEREGVSCISSSDASTYYADSVKGRFTISRDNAKKSVYLQMNSLKLEDTAVYYCAAGSLGYCSRSTPQYEYDYWGQGTQVTVSS

SEQ ID NO: 38 - protein, 8 aa, synthetic construct
GFSLEHWT

SEQ ID NO: 39 - protein, 8 aa, synthetic construct
ISSSDAST

SEQ ID NO: 40 - protein, 19 aa, synthetic construct
AAGSLGYCSRSTPQYEYDY

SEQ ID NO: 41 - protein, 126 aa, synthetic construct
QLQLVESGGGLVQPGGSLRLSCAASGFTLDYWTIGWFRQAPGKEREGVSCVSSSGGITYYADSVKGRFTISRDNEKRTVYLQMNSLKPEDTAVYYCAAGSLGYCSRSTPQYEYDYWGQGTQVTVSS

SEQ ID NO: 42 - protein, 8 aa, synthetic construct
GFTLDYWT

SEQ ID NO: 43 - protein, 9 aa, synthetic construct
VSSSGGITY

SEQ ID NO: 44 - protein, 19 aa, synthetic construct
AAGSLGYCSRSTPQYEYDY

SEQ ID NO: 45 - protein, 122 aa, synthetic construct
QVQLVESGGGLVQPGGSLRLSCAASGFSLDYWAIAWFRQAPGKEREGVSCSGSRGSTHYADSVKGRFTISRDNAKNTVYLQMNSLKPEDAGVYYCAVGSTLYSLTPYEYDYWGQGTQVTVSS

SEQ ID NO: 46 - protein, 8 aa, synthetic construct
GFSLDYWA

SEQ ID NO: 47 - protein, 8 aa, synthetic construct
SGSRGSTH

SEQ ID NO: 48 - protein, 16 aa, synthetic construct
AVGSTLYSLTPYEYDY

SEQ ID NO: 49 - protein, 122 aa, synthetic construct
QVQLVESGGGLVQPGGSLRLSCAASGFRLDHWAIGWFRQAPGKEREGVSCSGSDESTYYADSVKGRFTISRDNAKNTVYLQMNSLKPEDTGVYYCAVGSTWYSLTPYEYDYWGQGTQVTVSS

SEQ ID NO: 50 - protein, 8 aa, synthetic construct
GFRLDHWA

SEQ ID NO: 51 - protein, 8 aa, synthetic construct
SGSDESTY

SEQ ID NO: 52 - protein, 16 aa, synthetic construct
AVGSTWYSLTPYEYDY

SEQ ID NO: 53 - protein, 123 aa, synthetic construct
QVQLVESGGGLVQPGGSLRVSCAASGFRLDYYAIGWFRQAPGKEREGVSSINYSSGSTVYADSVKGRFTISRGNAKNTVYLQMNSLKPEDTAVYYCAVGTVVTSLAPYEYDYWGQGTQVTVSS

SEQ ID NO: 54 - protein, 8 aa, synthetic construct
GFRLDYYA

SEQ ID NO: 55 - protein, 11 aa, synthetic construct
SSINYSSGSTV

SEQ ID NO: 56 - protein, 16 aa, synthetic construct
AVGTVVTSLAPYEYDY

SEQ ID NO: 57 - protein, 126 aa, synthetic construct
QVQLVESGGGLVQPGGSLRLSCAASRFTLDYYAIGWIRQAPGKEREGVSCISSSGGSTNYADSVKGRFTISRDNAKNTVYLQMNSLKPEDTGVYYCAATTYACSGYVPRAYEYDYWGQGTQVTVSS

SEQ ID NO: 58 - protein, 9 aa, synthetic construct
SRFTLDYYA

SEQ ID NO: 59 - protein, 9 aa, synthetic construct
ISSSGGSTN

SEQ ID NO: 60 - protein, 19 aa, synthetic construct
AATTYACSGYVPRAYEYDY

SEQ ID NO: 61 - protein, 127 aa, synthetic construct
EVQLVESGGGLVQPGGSLRLSCALSGFTKDYYGIGWFRQAPGKEREGVSCISVSGSSTNYADSVKGRFTISRDNAKNTVYLQMNSLKPEDTGVYYCAAAFMGSDYVVPEMSREYDYWGQGTQVTVSS

SEQ ID NO: 62 - protein, 9 aa, synthetic construct
SGFTKDYYG

SEQ ID NO: 63 - protein, 9 aa, synthetic construct
ISVSGSSTN

SEQ ID NO: 64 - protein, 20 aa, synthetic construct
AAAFMGSDYVVPEMSREYDY

SEQ ID NO: 65 - protein, 125 aa, synthetic construct
EVQVVESGGGLVQPGGSLRLSCAVSGFTLDWYAIGWFRQAPGKEHEGVACIDPSERSTYADPVKGRFTISRDNAKNTVHLQMNSLKPEDTAVYYCAADTLPCFFYGSMWPPPDNWGQGTQVTVSS

SEQ ID NO: 66 - protein, 8 aa, synthetic construct
GFTLDWYA

SEQ ID NO: 67 - protein, 8 aa, synthetic construct
IDPSERST

SEQ ID NO: 68 - protein, 19 aa, synthetic construct
AADTLPCFFYGSMWPPPDN

SEQ ID NO: 69 - protein, 120 aa, synthetic construct
QVQLVESGGGLVQPGGSLRLSCAVSGDIFSFYSMGWYRQAPGEQRELVAHITSGGSTHYADSVKGRFTISRDNAKNTVYLQMNSPKPEDTAVYYCNADLDYWVDSMGPYWGQGTQVTVSS

SEQ ID NO: 70 - protein, 8 aa, synthetic construct
GDIFSFYS

SEQ ID NO: 71 - protein, 8 aa, synthetic construct
ITSGGSTH

SEQ ID NO: 72 - protein, 14 aa, synthetic construct
NADLDYWVDSMGPY

SEQ ID NO: 73 - protein, 115 aa, synthetic construct
QVQLVESGGGLVQPGGSLRLSCAASGFAFSFYVMTWYRQAPGKQREQVAAITSYGGANYADSVKGRFTISRDNAKNTVYLQMNALTSEDTAVYSCNAHLGPYDYWGQGTQVTVSS

SEQ ID NO: 74 - protein, 8 aa, synthetic construct
GFAFSFYV

SEQ ID NO: 75 - protein, 8 aa, synthetic construct
ITSYGGAN

SEQ ID NO: 76 - protein, 9 aa, synthetic construct
NAHLGPYDY

SEQ ID NO: 77 - protein, 122 aa, synthetic construct
EVQLVESGGGMVQPGGSLRLSCAFSGLTFSNYAMTWYRQAPGKERELVAGTPSPGGTTTYADSVKGRFTISRDNAEDTVYLQMNSLKPEDTAVYYCHARWMWPSDNAGMGYWGKGTLVTVSS

SEQ ID NO: 78 - protein, 8 aa, synthetic construct
GLTFSNYA

SEQ ID NO: 79 - protein, 9 aa, synthetic construct
TPSPGGTTT

SEQ ID NO: 80 - protein, 15 aa, synthetic construct
HARWMWPSDNAGMGY

SEQ ID NO: 81 - protein, 116 aa, synthetic construct
QVQLVESGGGLVQPGGSLRLSCAASSSIFSIAIMGWYRQAPGKQRELVATISATDVTHYADSVKGRFTISRDDAWNAVYLQMDSLKAEDTAVYYCNSYLLQQRVPWGQGTQVTVSS

SEQ ID NO: 82 - protein, 8 aa, synthetic construct
SSIFSIAI

SEQ ID NO: 83 - protein, 8 aa, synthetic construct
ISATDVTH

SEQ ID NO: 84 - protein, 10 aa, synthetic construct
NSYLLQQRVP

SEQ ID NO: 85 - protein, 116 aa, synthetic construct
EVQVVESGGGLVQPGGSLRLSCAASRSVFNIAIMGWYRQAPGKQRELVASISATDVTSYADSVKGRFTISRDDAWNAVYLQMDSLKSEDTAVYYCNSYLLQQRVPWGQGTQVTVSS

SEQ ID NO: 86 - protein, 8 aa, synthetic construct
RSVFNIAI

SEQ ID NO: 87 - protein, 8 aa, synthetic construct
ISATDVTS

SEQ ID NO: 88 - protein, 10 aa, synthetic construct
NSYLLQQRVP

SEQ ID NO: 89 - protein, 129 aa, synthetic construct
EVQVVESGGGLVQPGGSLRLSCAASGNIKSTAVMGWHRQAPGKEREWVAIITTGEDRFSGDRLQYADSVKDRFTISRDNAKNTVYLQMNSLKPEDTAVYYCNVDVLYMADGRYARSEFWGQGTQVTVSS

SEQ ID NO: 90 - protein, 8 aa, synthetic construct
GNIKSTAV

SEQ ID NO: 91 - protein, 14 aa, synthetic construct
ITTGEDRFSGDRLQ

SEQ ID NO: 92 - protein, 17 aa, synthetic construct
NVDVLYMADGRYARSEF

SEQ ID NO: 93 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 94 - protein, 8 aa, synthetic construct
GYTFTNYG

SEQ ID NO: 95 - protein, 8 aa, synthetic construct
INTYTGEP

SEQ ID NO: 96 - protein, 14 aa, synthetic construct
ARGGFGSSYWYFDV

SEQ ID NO: 97 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 98 - protein, 6 aa, synthetic construct
QDVSIA

SEQ ID NO: 99 - skipped

SEQ ID NO: 100 - protein, 9 aa, synthetic construct
QQHYITPLT

SEQ ID NO: 101 - protein, 243 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 102 - protein, 254 aa, synthetic construct
EVQLVESGGGLAQPGGSLRLSCAASGFTFSTYAMSWARQAPGKGLEWVSGIYADGSGTYYADSVKGRFTISRDNAKNTLYLQMNSLKPEDTALYYCAQGYSDYDRRLGEYDYWGQGTQVTVSSGGGGSGGGGSGGGGSDVQLQESGGGLVQAGGSLRLSCAASESIASINVMGWYRQAPGKQRELVAIITNRGTTSYPDSVKGRFTISRDNGKNTVYLQMNTLKPEDTAVYYCNAPWTDGINYWGKGTQVTVSS

SEQ ID NO: 103 - protein, 253 aa, synthetic construct
QVQLVESGGGLVQPGGSLRLSCAASGFSLDYWAIAWFRQAPGKEREGVSCSGSRGSTHYADSVKGRFTISRDNAKNTVYLQMNSLKPEDAGVYYCAVGSTLYSLTPYEYDYWGQGTQVTVSSGGGGSGGGGSGGGGSDVQLQESGGGLVQAGGSLRLSCAASESIASINVMGWYRQAPGKQRELVAIITNRGTTSYPDSVKGRFTISRDNGKNTVYLQMNTLKPEDTAVYYCNAPWTDGINYWGKGTQVTVSS

SEQ ID NO: 104 - protein, 251 aa, synthetic construct
QVQLVESGGGLVQPGGSLRLSCAVSGDIFSFYSMGWYRQAPGEQRELVAHITSGGSTHYADSVKGRFTISRDNAKNTVYLQMNSPKPEDTAVYYCNADLDYWVDSMGPYWGQGTQVTVSSGGGGSGGGGSGGGGSDVQLQESGGGLVQAGGSLRLSCAASESIASINVMGWYRQAPGKQRELVAIITNRGTTSYPDSVKGRFTISRDNGKNTVYLQMNTLKPEDTAVYYCNAPWTDGINYWGKGTQVTVSS

SEQ ID NO: 105 - protein, 254 aa, synthetic construct
EVQLVESGGGLAQPGGSLRLSCAASGFTFSTYAMSWARQAPGKGLEWVSGIYADGSGTYYADSVKGRFTISRDNAKNTLYLQMNSLKPEDTALYYCAQGYSDYDRRLGEYDYWGQGTQVTVSSGGGGSGGGGSGGGGSDVQLQESGGGLVQAGDSLGLSCAASGRAFSLFEMAWYRQAPGKAREFVAAIMWRGGHTVYADSVKGRSTISSDNTKNTVYLQMNSLKPEDTAIYYCRGKVVGSDYWGQGTQVTVSS

SEQ ID NO: 106 - protein, 253 aa, synthetic construct
QVQLVESGGGLVQPGGSLRLSCAASGFSLDYWAIAWFRQAPGKEREGVSCSGSRGSTHYADSVKGRFTISRDNAKNTVYLQMNSLKPEDAGVYYCAVGSTLYSLTPYEYDYWGQGTQVTVSSGGGGSGGGGSGGGGSDVQLQESGGGLVQAGDSLGLSCAASGRAFSLFEMAWYRQAPGKAREFVAAIMWRGGHTVYADSVKGRSTISSDNTKNTVYLQMNSLKPEDTAIYYCRGKVVGSDYWGQGTQVTVSS

SEQ ID NO: 107 - protein, 251 aa, synthetic construct
QVQLVESGGGLVQPGGSLRLSCAVSGDIFSFYSMGWYRQAPGEQRELVAHITSGGSTHYADSVKGRFTISRDNAKNTVYLQMNSPKPEDTAVYYCNADLDYWVDSMGPYWGQGTQVTVSSGGGGSGGGGSGGGGSDVQLQESGGGLVQAGDSLGLSCAASGRAFSLFEMAWYRQAPGKAREFVAAIMWRGGHTVYADSVKGRSTISSDNTKNTVYLQMNSLKPEDTAIYYCRGKVVGSDYWGQGTQVTVSS

SEQ ID NO: 108 - skipped

SEQ ID NO: 109 - protein, 8 aa, synthetic construct
  VARIANT 1: note = I or V
  VARIANT 7: note = A, G, V, L or I
XYADGSXT

SEQ ID NO: 110 - protein, 16 aa, synthetic construct
  VARIANT 11: note = L, V, I, M or A
  VARIANT 13: note = E, G, A, D or N
  VARIANT 16: note = Y or F
AQGYSDYDRRXGXYDX

SEQ ID NO: 111 - skipped

SEQ ID NO: 112 - protein, 8 aa, synthetic construct
  VARIANT 1: note = I or V
  VARIANT 3: note = S or R
  VARIANT 6: note = G, S or A
  VARIANT 7: note = T, S or I
XSXSDXXT

SEQ ID NO: 113 - skipped

SEQ ID NO: 114 - protein, 19 aa, synthetic construct
  VARIANT 3: note = G, D or A
  VARIANT 16: note = E, G, A, D or N
  VARIANT 19: note = Y or F
AAXSLGYCSRSTPQYXYDX

SEQ ID NO: 115 - protein, 8 aa, synthetic construct
  VARIANT 4: note = D, R or S
  VARIANT 5: note = E or G
  VARIANT 8: note = Y, H or V
SGSXXSTX

SEQ ID NO: 116 - protein, 16 aa, synthetic construct
  VARIANT 4: note = S or T
  VARIANT 5: note = T or V
  VARIANT 6: note = L, W or V
  VARIANT 7: note = Y or T
  VARIANT 10: note = A or T
  VARIANT 13: note = E, G, A, D or N
AVGXXXXSLXPYXYDY

SEQ ID NO: 117 - skipped

SEQ ID NO: 118 - protein, 9 aa, synthetic construct
  VARIANT 3: note = S or V
  VARIANT 6: note = S or G
ISXSGXSTN

SEQ ID NO: 119-120 - skipped (2 entries)

SEQ ID NO: 121 - protein, 8 aa, synthetic construct
  VARIANT 2: note = D or F
  VARIANT 3: note = I, A or T
  VARIANT 8: note = S or V
GXXFSFYX

SEQ ID NO: 122 - skipped

SEQ ID NO: 123 - protein, 14 aa, synthetic construct
  VARIANT 1: note = N or T
XADLDYWVDSMGPY

SEQ ID NO: 124 - protein, 8 aa, synthetic construct
  VARIANT 4: note = Y or G
  VARIANT 6: note = G or S
  VARIANT 7: note = A, T or S
  VARIANT 8: note = N or T
ITSXGXXX

SEQ ID NO: 125 - skipped

SEQ ID NO: 126 - protein, 8 aa, synthetic construct
  VARIANT 1: note = S or R
  VARIANT 3: note = I or V
  VARIANT 5: note = S or N
XSXFXIAI

SEQ ID NO: 127 - protein, 8 aa, synthetic construct
  VARIANT 8: note = H or S
ISATDVTX

SEQ ID NO: 128 - DNA, 20 bp, synthetic construct
  source 1..20: mol_type = other DNA
gcttctgctgccggttaacg

SEQ ID NO: 129 - DNA, 20 bp, synthetic construct
  source 1..20: mol_type = other DNA
acagtgatcacactccatgt

SEQ ID NO: 130 - protein, 272 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 131 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 132 - DNA, 486 bp, Homo sapiens
  source 1..486: mol_type = genomic DNA
atgagaatttccaaaccacatttgaggagcatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcacattgatgctactttatatacggaaagtgatgttcaccccagttgcaaagtaacagccatgaagtgctttctcttggagttacaagttatttcacttgagagcggagatgcaagtattcatgatacagtagaaaatctgatcatcctagcaaacaacagtttgtcttctaatgggaatgtaacagaatctggatgcaaagaatgtgaggaactggaggagaagaacatcaaggaatttttgcagagttttgtgcatattgtccaaatgttcatcaacacttct

SEQ ID NO: 133 - protein, 248 aa, synthetic construct
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 134 - skipped

SEQ ID NO: 135 - protein, 8 aa, synthetic construct
  VARIANT 2: note = F, R or S
  VARIANT 3: note = W, T, S or R
  VARIANT 4: note = F or L
GXXXNHYG

SEQ ID NO: 136 - protein, 8 aa, synthetic construct
  VARIANT 2: note = F, R or S
  VARIANT 3: note = W, T, S or R
  VARIANT 4: note = F or L
GXXXSLFE

SEQ ID NO: 137 - protein, 45 aa, synthetic construct
TTTPAPRPPTPAPTIASQPLSLRPEACRPAAGGAVHTRGLDFACD

SEQ ID NO: 138 - protein, 24 aa, synthetic construct
IYIWAPLAGTCGVLLLSLVITLYC

SEQ ID NO: 139 - protein, 42 aa, synthetic construct
KRGRKKLLYIFKQPFMRPVQTTQEEDGCSCRFPEEEEGGCEL

SEQ ID NO: 140 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 18 aa, synthetic construct
EGRGSLLTCGDVEENPGP

SEQ ID NO: 142 - DNA, 20 bp, synthetic construct
  source 1..20: mol_type = other DNA
tctactgctaccgcgttaac